SEQUENCE LISTING (223 sequences)

SEQ ID NO: 1 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAALVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 2 - protein, 339 aa, Mus musculus
MEISDDVTNPEQLWKMKPKGNLEDDSYSTKDSGETSMLKRPGLSHLQHAVHVDAFDCPSELQHTQEFFPNWRLPVKVAAIISSLTFLYTLLREIIYPLVTSREQYFYKIPILVINKVLPMVAITLLALVYLPGELAAVVQLRNGTKYKKFPPWLDRWMLARKQFGLLSFFFAVLHAVYSLSYPMRRSYRYKLLNWAYKQVQQNKEDAWVEHDVWRMEIYVSLGIVGLAILALLAVTSIPSVSDSLTWREFHYIQSKLGIVSLLLGTVHALVFAWNKWVDVSQFVWYMPPTFMIAVFLPTLVLICKIALCLPCLRKKILKIRCGWEDVSKINRTEMASRL

SEQ ID NO: 3 - protein, 336 aa, Canis lupus
  source 1..336: note = Canis lupus familiaris
MESRQDITSQEELWTMKPRRNLEEDDYLDKDSGDTRVLKRPVLLHMHQTTHFDEFDCPAELKHKQELFPMWRWPVKIAAVISSLTFLYTLLREIIHPFVTSHQQYFYKIPILVINKVLPMVSITLLALVYLPGVIAAVVQLHNGTKYKKFPHWLDRWMLTRKQFGLLSFFFAVLHAIYSLSYPMRRSYRYKLLNWAYQQVQQNKEDAWIEHDVWRMEIYVSLGIVTLAILALLAVTSIPSVSDSLTWREFHYIQSKLGMVSLLLGTIHALIFAWNKWVDIKQFVWYTPPTFMIAVFLPIVVLICKAILFLPCLRKKILKIRHGWEDVTKINKTEMS

SEQ ID NO: 4 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 5 - DNA, 471 bp, Homo sapiens
  source 1..471: mol_type = genomic DNA
tactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 6 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 7 - protein, 271 aa, Homo sapiens [sequence omitted]

SEQ ID NO: 8 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 9 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 10 - protein, 158 aa, Homo sapiens
MEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 11 - protein, 164 aa, Homo sapiens
MNPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 12 - protein, 157 aa, Homo sapiens
MNPQREAAPKSYAIRDSRQMVWVLSGNSLIAAPLSRSIKPVTLHLIACRDTEFSDKEKGNMVYLGIKGKDLCLFCAEIQGKPTLQLKEKNIMDLYVEKKAQKPFLFFHNKEGSTSVFQSVSYPGWFIATSTTSGQPIFLTKERGITNNTNFYLDSVE

SEQ ID NO: 13 - protein, 169 aa, Homo sapiens
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 14 - protein, 6 aa, Synthetic construct
SDYAWN

SEQ ID NO: 15 - protein, 16 aa, Synthetic construct
YISNSGSTSYNPSLKS

SEQ ID NO: 16 - protein, 15 aa, Synthetic construct
ERNYDYDDYYYAMDY

SEQ ID NO: 17 - protein, 17 aa, Synthetic construct
KSSQSLLYRSNQKNYLA

SEQ ID NO: 18 - protein, 7 aa, Synthetic construct
WASTRES

SEQ ID NO: 19 - protein, 9 aa, Synthetic construct
QQYYNYPRT

SEQ ID NO: 20 - protein, 8 aa, Synthetic construct
GYSITSDY

SEQ ID NO: 21 - protein, 5 aa, Synthetic construct
SNSGS

SEQ ID NO: 22 - protein, 9 aa, Synthetic construct
GYSITSDYA

SEQ ID NO: 23 - protein, 7 aa, Synthetic construct
ISNSGST

SEQ ID NO: 24 - protein, 17 aa, Synthetic construct
ARERNYDYDDYYYAMDY

SEQ ID NO: 25 - protein, 12 aa, Synthetic construct
QSLLYRSNQKNY

SEQ ID NO: 26 - protein, 14 aa, Synthetic construct
AVSGYSITSDYAWN

SEQ ID NO: 27 - protein, 9 aa, Synthetic construct
YISNSGSTS

SEQ ID NO: 28 - protein, 8 aa, Synthetic construct
YWASTRES

SEQ ID NO: 29 - protein, 7 aa, Synthetic construct
TSDYAWN

SEQ ID NO: 30 - protein, 12 aa, Synthetic construct
WVGYISNSGSTS

SEQ ID NO: 31 - protein, 16 aa, Synthetic construct
ARERNYDYDDYYYAMD

SEQ ID NO: 32 - protein, 7 aa, Synthetic construct
KNYLAWY

SEQ ID NO: 33 - protein, 10 aa, Synthetic construct
LLIYWASTRE

SEQ ID NO: 34 - protein, 8 aa, Synthetic construct
QQYYNYPR

SEQ ID NO: 35 - protein, 124 aa, Synthetic construct
EVQLVESGGGLVQPGGSLRLSCAVSGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSS

SEQ ID NO: 36 - protein, 114 aa, Synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKR

SEQ ID NO: 37 - protein, 5 aa, Synthetic construct
  REPEAT 1..5: note = (GGGGS) is repeated n times, wherein n is an integer including, 1, 2, 3, 4, 5, 6, 7, 8, 9, or more
GGGGS

SEQ ID NO: 38 - protein, 9 aa, Synthetic construct
GGGSGGGGS

SEQ ID NO: 39 - protein, 7 aa, Synthetic construct
GGGSGGS

SEQ ID NO: 40 - protein, 9 aa, Synthetic construct
GGGSGGGGS

SEQ ID NO: 41 - protein, 20 aa, Synthetic construct
GGGGSGGGGSGGGGSGGGGS

SEQ ID NO: 42 - protein, 15 aa, Synthetic construct
GGGGSGGGGSGGGGS

SEQ ID NO: 43 - protein, 10 aa, Synthetic construct
GGGGSGGGGS

SEQ ID NO: 44 - protein, 5 aa, Synthetic construct
GGGGS

SEQ ID NO: 45 - protein, 8 aa, Synthetic construct
GGGSGGGS

SEQ ID NO: 46 - protein, 4 aa, Synthetic construct
GGGS

SEQ ID NO: 47 - protein, 6 aa, Synthetic construct
GGSGGS

SEQ ID NO: 48 - protein, 11 aa, Synthetic construct
GGSGGGSGGSG

SEQ ID NO: 49 - protein, 10 aa, Synthetic construct
GGSGGGSGSG

SEQ ID NO: 50 - protein, 8 aa, Synthetic construct
GGSGGGSG

SEQ ID NO: 51 - protein, 5 aa, Synthetic construct
  REPEAT 1..5: note = (EAAAK) is repeated n times, wherein n is an integer including 1, 2, 3, 4, 5, 6, 7, 8, 9, or more.
EAAAK

SEQ ID NO: 52 - protein, 6 aa, Synthetic construct
HHHHHH

SEQ ID NO: 53 - protein, 8 aa, Synthetic construct
DYKDDDDK

SEQ ID NO: 54 - protein, 8 aa, Synthetic construct
DLYDDDDK

SEQ ID NO: 55 - protein, 15 aa, Synthetic construct
GLNDIFEAQKIEWHE

SEQ ID NO: 56 - protein, 26 aa, Synthetic construct
KRRWKKNFIAVSAANRFKKISSSGAL

SEQ ID NO: 57 - protein, 9 aa, Synthetic construct
YPYDVPDYA

SEQ ID NO: 58 - protein, 10 aa, Synthetic construct
EQKLISEEDL

SEQ ID NO: 59 - protein, 8 aa, Synthetic construct
WRHPQFGG

SEQ ID NO: 60 - protein, 8 aa, Synthetic construct
WSHPQFEK

SEQ ID NO: 61 - protein, 13 aa, Synthetic construct
SLAELLNAGLGGS

SEQ ID NO: 62 - protein, 8 aa, Synthetic construct
TQDPSRVG

SEQ ID NO: 63 - protein, 14 aa, Synthetic construct
GKPIPNPLLGLDST

SEQ ID NO: 64 - DNA, 20 bp, Synthetic construct
  source 1..20: mol_type = other DNA
atagtctgtcttacccaatg

SEQ ID NO: 65 - DNA, 20 bp, Synthetic construct
  source 1..20: mol_type = other DNA
cctttgtagcataaggacac

SEQ ID NO: 66 - DNA, 20 bp, Synthetic construct
  source 1..20: mol_type = other DNA
atccacttatccaaccaatg

SEQ ID NO: 67 - DNA, 20 bp, Synthetic construct
  source 1..20: mol_type = other DNA
catcaacaaagtcttgccaa

SEQ ID NO: 68 - protein, 33 aa, Homo sapiens
RRSYRYKLLNWAYQQVQQNKEDAWIEHDVWRME

SEQ ID NO: 69 - protein, 33 aa, Mus musculus
RRSYRYKLLNWAYKQVQQNKEDAWVEHDVWRME

SEQ ID NO: 70 - DNA, 342 bp, Synthetic construct
  source 1..342: mol_type = other DNA
gatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacgg

SEQ ID NO: 71 - DNA, 45 bp, Synthetic construct
  source 1..45: mol_type = other DNA
ggtggtggtggctctgggggtggcggtagcggaggtggaggatca

SEQ ID NO: 72 - DNA, 372 bp, Synthetic construct
  source 1..372: mol_type = other DNA
gaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttca

SEQ ID NO: 73 - protein, 253 aa, Synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRGGGGSGGGGSGGGGSEVQLVESGGGLVQPGGSLRLSCAVSGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSS

SEQ ID NO: 74 - DNA, 759 bp, Synthetic construct
  source 1..759: mol_type = other DNA
gatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacggggtggtggtggctctgggggtggcggtagcggaggtggaggatcagaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttca

SEQ ID NO: 75 - protein, 253 aa, Synthetic construct
EVQLVESGGGLVQPGGSLRLSCAVSGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKR

SEQ ID NO: 76 - DNA, 759 bp, Synthetic construct
  source 1..759: mol_type = other DNA
gaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttcaggtggtggtggctctgggggtggcggtagcggaggtggaggatcagatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacgg

SEQ ID NO: 77 - protein, 12 aa, Synthetic construct
ESKYGPPCPPCP

SEQ ID NO: 78 - DNA, 36 bp, Synthetic construct
  source 1..36: mol_type = other DNA
gaatctaagtacggaccgccctgccccccttgccct

SEQ ID NO: 79 - DNA, 36 bp, Synthetic construct
  source 1..36: mol_type = other DNA
gagtctaagtacggaccgccttgcccaccgtgccca

SEQ ID NO: 80 - DNA, 36 bp, Synthetic construct
  source 1..36: mol_type = other DNA
gagtctaagtacggaccgccctgccccccttgccct

SEQ ID NO: 81 - protein, 108 aa, Synthetic construct
APPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKA

SEQ ID NO: 82 - DNA, 326 bp, Synthetic construct
  source 1..326: mol_type = other DNA
gccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaa

SEQ ID NO: 83 - DNA, 321 bp, Synthetic construct
  source 1..321: mol_type = other DNA
ggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 84 - protein, 108 aa, Synthetic construct
KGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - DNA, 323 bp, Synthetic construct
  source 1..323: mol_type = other DNA
agggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaag

SEQ ID NO: 86 - DNA, 359 bp, Synthetic construct
  source 1..359: mol_type = other DNA
gaatctaagtacggaccgccctgccccccttgccctagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaag

SEQ ID NO: 87 - protein, 228 aa, Synthetic construct
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - DNA, 685 bp, Synthetic construct
  source 1..685: mol_type = other DNA
gaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaag

SEQ ID NO: 89 - protein, 115 aa, Synthetic construct
GGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 345 bp, Synthetic construct
  source 1..345: mol_type = other DNA
ggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 92 - DNA, 336 bp, Synthetic construct
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 93 - protein, 42 aa, Synthetic construct
KRGRKKLLYIFKQPFMRPVQTTQEEDGCSCRFPEEEEGGCEL

SEQ ID NO: 94 - protein, 43 aa, Synthetic construct
VKRGRKKLLYIFKQPFMRPVQTTQEEDGCSCRFPEEEEGGCEL

SEQ ID NO: 95 - protein, 41 aa, Synthetic construct
VKRGRKKLLYIFKQPFMRPVQTTQEEDGCSCRFPEEEEGGC

SEQ ID NO: 96 - DNA, 126 bp, Synthetic construct
  source 1..126: mol_type = other DNA
aagagaggacggaagaagctgctgtatatctttaagcagccttttatgagacctgtccaaacaacccaagaggaggacgggtgttcctgtcgcttcccagaggaggaggaaggagggtgcgagctg

SEQ ID NO: 97 - DNA, 126 bp, Synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 98 - DNA, 129 bp, Synthetic construct
  source 1..129: mol_type = other DNA
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 99 - protein, 28 aa, Synthetic construct
MFWVLVVVGGVLACYSLLVTVAFIIFWV

SEQ ID NO: 100 - protein, 27 aa, Synthetic construct
MFWVLVVVGGVLACYSLLVTVAFIIFW

SEQ ID NO: 101 - protein, 26 aa, Synthetic construct
FWVLVVVGGVLACYSLLVTVAFIIFW

SEQ ID NO: 102 - DNA, 84 bp, Synthetic construct
  source 1..84: mol_type = other DNA
atgttctgggtgctggtggtggtgggcggggtgctggcctgctacagcctgctggtgacagtggccttcatcatcttttgggta

SEQ ID NO: 103 - DNA, 84 bp, Synthetic construct
  source 1..84: mol_type = other DNA
atgttctgggtgctggtggtggtcggaggcgtgctggcctgctacagcctgctggtcaccgtggccttcatcatcttttgggtg

SEQ ID NO: 104 - DNA, 81 bp, Synthetic construct
  source 1..81: mol_type = other DNA
atgttctgggtgctggtggtggtcggaggcgtgctggcctgctacagcctgctggtcaccgtggccttcatcatcttttgg

SEQ ID NO: 105 - DNA, 81 bp, Synthetic construct
  source 1..81: mol_type = other DNA
atgttctgggtgctggtggtggtgggcggggtgctggcctgctacagcctgctggtgacagtggccttcatcatcttttgg

SEQ ID NO: 106 - protein, 357 aa, Synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 107 - DNA, 1071 bp, Synthetic construct
  source 1..1071: mol_type = other DNA
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 108 - DNA, 972 bp, Synthetic construct
  source 1..972: mol_type = other DNA
atgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 109 - protein, 22 aa, Synthetic construct
GSGEGRGSLLTCGDVEENPGPR

SEQ ID NO: 110 - protein, 24 aa, Synthetic construct
LEGGGEGRGSLLTCGDVEENPGPR

SEQ ID NO: 111 - protein, 21 aa, Synthetic construct
GSGEGRGSLLTCGDVEENPGP

SEQ ID NO: 112 - DNA, 66 bp, Synthetic construct
  source 1..66: mol_type = other DNA
ggctccggcgagggcagaggaagtcttctaacatgcggtgacgtggaggagaatcccggccctagg

SEQ ID NO: 113 - DNA, 72 bp, Synthetic construct
  source 1..72: mol_type = other DNA
ctcgagggcggcggagagggcagaggaagtcttctaacatgcggtgacgtggaggagaatcccggccctagg

SEQ ID NO: 114 - DNA, 72 bp, Synthetic construct
  source 1..72: mol_type = other DNA
ctcgagggcggcggagagggcagaggaagtcttctaacatgcggtgacgtggaggagaatccaggccctagg

SEQ ID NO: 115 - protein, 19 aa, Synthetic construct
ATNFSLLKQAGDVEENPGP

SEQ ID NO: 116 - protein, 22 aa, Synthetic construct
GSGATNFSLLKQAGDVEENPGP

SEQ ID NO: 117 - DNA, 57 bp, Synthetic construct
  source 1..57: mol_type = other DNA
gccacaaacttctctctgctaaagcaagcaggtgatgttgaagaaaaccccgggcct

SEQ ID NO: 118 - protein, 23 aa, Synthetic construct
GSGQCTNYALLKLAGDVESNPGP

SEQ ID NO: 119 - protein, 25 aa, Synthetic construct
GSGVKQTLNFDLLKLAGDVESNPGP

SEQ ID NO: 120 - protein, 20 aa, Synthetic construct
MYRMQLLSCIALSLALVTNS

SEQ ID NO: 121 - DNA, 60 bp, Synthetic construct
  source 1..60: mol_type = other DNA
atgtacagaatgcaactcctgtcttgtattgcactaagtctcgcacttgtcacaaacagt

SEQ ID NO: 122 - DNA, 529 bp, Synthetic construct
  source 1..529: mol_type = other DNA
agttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 123 - DNA, 544 bp, Synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 124 - DNA, 545 bp, Synthetic construct
  source 1..545: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacg

SEQ ID NO: 125 - DNA, 138 bp, Synthetic construct
  source 1..138: mol_type = other DNA
tcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggag

SEQ ID NO: 126 - DNA, 113 bp, Synthetic construct
  source 1..113: mol_type = other DNA
ctcggcttgctgaagcgcgcacggcaagaggcgaggggcggcgactggtgagtacgccaaaaattttgactagcggaggctagaaggagagagatgggtgcgagagcgtcagt

SEQ ID NO: 127 - DNA, 200 bp, Synthetic construct
  source 1..200: mol_type = other DNA
gcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggc

SEQ ID NO: 128 - DNA, 333 bp, Synthetic construct
  source 1..333: mol_type = other DNA
gcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatc

SEQ ID NO: 129 - DNA, 179 bp, Synthetic construct
  source 1..179: mol_type = other DNA
tacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgg

SEQ ID NO: 130 - DNA, 66 bp, Synthetic construct
  source 1..66: mol_type = other DNA
atgctgctgctggtgaccagcctgctgctgtgcgagctgccccaccccgcctttctgctgatcccc

SEQ ID NO: 131 - DNA, 66 bp, Synthetic construct
  source 1..66: mol_type = other DNA
atgctgctgctcgtgaccagcctgctgctgtgcgaactgccccaccctgcctttctgctgatcccc

SEQ ID NO: 132 - DNA, 588 bp, Synthetic construct
  source 1..588: mol_type = other DNA
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 133 - DNA, 601 bp, Synthetic construct
  source 1..601: mol_type = other DNA
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgat

SEQ ID NO: 134 - DNA, 53 bp, Synthetic construct
  source 1..53: mol_type = other DNA
tggaagggctaattcactcccaacgaagacaagatctgctttttgcttgtact

SEQ ID NO: 135 - DNA, 105 bp, Synthetic construct
  source 1..105: mol_type = other DNA
gtacctttaagaccaatgacttacaaggcagctgtagatcttagccactttttaaaagaaaaggggggactggaagggctaattcactcccaaagaagacaagat

SEQ ID NO: 136 - DNA, 96 bp, Synthetic construct
  source 1..96: mol_type = other DNA
gggtctctctggttagaccagatctgagcctgggagctctctggctaactagggaacccactgcttaagcctcaataaagcttgccttgagtgctt

SEQ ID NO: 137 - DNA, 81 bp, Synthetic construct
  source 1..81: mol_type = other DNA
ctgctttttgcctgtactgggtctctctggttagaccagatctgagcctgggagctctctggctaactagggaacccactg

SEQ ID NO: 138 - DNA, 85 bp, Synthetic construct
  source 1..85: mol_type = other DNA
caagtagtgtgtgcccgtctgttgtgtgactctggtaactagagatccctcagacccttttagtcagtgtggaaaatctctagca

SEQ ID NO: 139 - DNA, 114 bp, Synthetic construct
  source 1..114: mol_type = other DNA
cttaagcctcaataaagcttgccttgagtgcttcaagtagtgtgtgcccgtctgttgtgtgactctggtaactagagatccctcagacccttttagtcagtgtggaaaatctct

SEQ ID NO: 140 - DNA, 660 bp, Synthetic construct
  source 1..660: mol_type = other DNA
atgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 141 - DNA, 859 bp, Synthetic construct
  source 1..859: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggt

SEQ ID NO: 142 - DNA, 683 bp, Synthetic construct
  source 1..683: mol_type = other DNA
tcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 143 - DNA, 882 bp, Synthetic construct
  source 1..882: mol_type = other DNA
aaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagag

SEQ ID NO: 144 - DNA, 23 bp, Synthetic construct
  source 1..23: mol_type = other DNA
ggaattgtgagcggataacaatt

SEQ ID NO: 145 - DNA, 21 bp, Synthetic construct
  source 1..21: mol_type = other DNA
caggaaacagctatgaccatg

SEQ ID NO: 146 - DNA, 20 bp, Synthetic construct
  source 1..20: mol_type = other DNA
attaaccctcactaaaggga

SEQ ID NO: 147 - DNA, 180 bp, Synthetic construct
  source 1..180: mol_type = other DNA
cacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggg

SEQ ID NO: 148 - DNA, 536 bp, Synthetic construct
  source 1..536: mol_type = other DNA
atcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatc

SEQ ID NO: 149 - DNA, 21 bp, Synthetic construct
  source 1..21: mol_type = other DNA
ccctatagtgagtcgtattac

SEQ ID NO: 150 - DNA, 18 bp, Synthetic construct
  source 1..18: mol_type = other DNA
actggccgtcgttttaca

SEQ ID NO: 151 - DNA, 69 bp, Synthetic construct
  source 1..69: mol_type = other DNA
tggcgtaatagcgaagaggcccgcaccgatcgcccttcccaacagttgcgcagcctgaatggcgaatgg

SEQ ID NO: 152 - DNA, 456 bp, Synthetic construct
  source 1..456: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaattt

SEQ ID NO: 153 - DNA, 200 bp, Synthetic construct
  source 1..200: mol_type = other DNA
ggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 154 - DNA, 18 bp, Synthetic construct
  source 1..18: mol_type = other DNA
catcatcaccaccatcac

SEQ ID NO: 155 - protein, 353 aa, Synthetic construct
PEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPET

SEQ ID NO: 156 - DNA, 1059 bp, Synthetic construct
  source 1..1059: mol_type = other DNA
ccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccttggcacgctgggacttattcctgtgtggcagaaaacattcttggtactggacagaggggccctggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccaggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagacc

SEQ ID NO: 157 - protein, 179 aa, Synthetic construct
PRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPET

SEQ ID NO: 158 - DNA, 537 bp, Synthetic construct
  source 1..537: mol_type = other DNA
ccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccaggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagacc

SEQ ID NO: 159 - protein, 51 aa, Synthetic construct
RSLRECELYVQKHNIQALLKDSIVQLCTARPERPMAFLREYFERLEKEEAK

SEQ ID NO: 160 - protein, 50 aa, Synthetic construct
SLRECELYVQKHNIQALLKDSIVQLCTARPERPMAFLREYFERLEKEEAK

SEQ ID NO: 161 - protein, 50 aa, Synthetic construct
SLKGCELYVQLHGIQQVLKDCIVHLCISKPERPMKFLREHFEKLEKEENR

SEQ ID NO: 162 - protein, 51 aa, Synthetic construct
RSLRECELYVQKHNIQALLKDRIVQLCTARPERPMAFLREYFERLEKEEAK

SEQ ID NO: 163 - protein, 50 aa, Synthetic construct
SLRECELYVQKHNIQALLKDRIVQLCTARPERPMAFLREYFERLEKEEAK

SEQ ID NO: 164 - protein, 50 aa, Synthetic construct
SLRECELYVQKHNIQALLKDEIVQLCTARPERPMAFLREYFERLEKEEAK

SEQ ID NO: 165 - protein, 44 aa, Synthetic construct
SHIQIPPGLTELLQGYTVEVLRQQPPDLVEFAVEYFTRLREARA

SEQ ID NO: 166 - protein, 45 aa, Synthetic construct
CGHIQIPPGLTELLQGYTVEVLRQQPPDLVEFAVEYFTRLREARA

SEQ ID NO: 167 - protein, 17 aa, Synthetic construct
QIEYLAKQIVDNAIQQA

SEQ ID NO: 168 - protein, 21 aa, Synthetic construct
CGQIEYLAKQIVDNAIQQAGC

SEQ ID NO: 169 - protein, 39 aa, Synthetic construct
RIARLEEKVKTLKAQNSELASTANMLREQVAQLKQKVMN

SEQ ID NO: 170 - protein, 39 aa, Synthetic construct
LTDTLQAETDQLEDKKSALQTEIANLLKEKEKLEFILAA

SEQ ID NO: 171 - protein, 26 aa, Synthetic construct
  VARIANT 10: note = K or R
  VARIANT 12: note = Y or F
  VARIANT 13: note = Y or F
  VARIANT 18: note = T or S
  VARIANT 19: note = K or R
  VARIANT 21: note = T or S
  VARIANT 22: note = T or Q or S
WXXXXXXXGXXXXXBXXXXXXXWXXP

SEQ ID NO: 172 - protein, 59 aa, Synthetic construct
WREFCESHARAAALDFARRFRLYLASHPQYAGPGAEAAFSRRFAELFLQHFEAEVARAS

SEQ ID NO: 173 - protein, 55 aa, Synthetic construct
THRAIFRFVPRHEDELELEVDDPLLVELQAEDYWYEAYNMRTGARGVFPAYYAIE

SEQ ID NO: 174 - protein, 101 aa, Synthetic construct
LGEVHRFHVTVAPGTKLESGPATLHLCNDVLVLARDIPPAVTGQWKLSDLRRYGAVPSGFIFEGGTRCGYWAGVFFLSSAEGEQISFLFDCIVRGISPTKG

SEQ ID NO: 175 - protein, 115 aa, Synthetic construct
DCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSS

SEQ ID NO: 176 - protein, 427 aa, Synthetic construct
CAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGSQLFSYAAFSDSNIITVVVDTALYIAKQNSPVVEVWDKKTEKLCGLIDCVHFLREVMVKETKIFSFSNDFTIQKLIETRTNKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 177 - protein, 75 aa, Synthetic construct
DQELKHLILEAADGFLFIVSCETGRVVYVSDSVTPVLNQQQSEWFGSTLYDQVHPDDVDKLREQLSTSENALTGR

SEQ ID NO: 178 - protein, 33 aa, Synthetic construct
LSAKETKMLMAAGDKDGDGKIGVDEFSTLVAES

SEQ ID NO: 179 - protein, 106 aa, Synthetic construct
MGVQVETISPGDGRTFPKRGQTCWHYTGMLEDGKKFDSSRDRNPFKFMLGKQEVIRGWEEGVAQMSVGQRAKLTISPDYAYGATGHPGIIPPHATLVFDVELLKLE

SEQ ID NO: 180 - protein, 100 aa, Synthetic construct
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 181 - protein, 68 aa, Synthetic construct
SGRAHAGWETPEGCEQVLTGKRLMQCLPNPEDVKMALEVYKLSLEIEQLELQRDSARQSTLDKELVPR

SEQ ID NO: 182 - protein, 55 aa, Synthetic construct
KKQGDADVCGEVAYIQSVVSDCHVPTAELRTLLEIRKLFLEIQKLKVELQGLSKE

SEQ ID NO: 183 - protein, 57 aa, Synthetic construct
ETPEGCEQVLTGKRLMQCLPNPEDVKMALEVYKLSLEIEQLELQRDSARQSTLDKEL

SEQ ID NO: 184 - protein, 58 aa, Synthetic construct
WETPEGCEQVLTGKRLMQCLPNPEDVKMALEVYKLSLEIEQLELQRDSARQSTLDKEL

SEQ ID NO: 185 - protein, 52 aa, Synthetic construct
CEQVLTGKRLMQCLPNPEDVKMALEVYKLSLEIEQLELQRDSARQSTLDKEL

SEQ ID NO: 186 - protein, 57 aa, Synthetic construct
ETPEGCEQVLTGKRLMQCLPNPEDVKMALEVYKLSLEIEQLELQRDSARQYTLDKEL

SEQ ID NO: 187 - protein, 57 aa, Synthetic construct
ETPEGCEQVLAGKRLMQCLPNPEDVKMALEVYKLSLEIEQLELQRDRARQSTLDKEL

SEQ ID NO: 188 - protein, 57 aa, Synthetic construct
ETPEGCEQVLAGKRLMQCLPNPEDVKMALEVYKLSLEIEQLELQRDRARQSTWDKEL

SEQ ID NO: 189 - protein, 57 aa, Synthetic construct
EVPEGCEQVQAGRRLMQCLADPYEVKMALEVYKLSLEIELLELQRDKARKSSVLRQL

SEQ ID NO: 190 - protein, 57 aa, Synthetic construct
VVPEGCEHILKGRKTMQCLPNPEDVKMALEIYKLSLDIELLELQRDRAKESTVQSPV

SEQ ID NO: 191 - protein, 55 aa, Synthetic construct
EVPKDCEHVFAGKKLMQCLPNSNDVKMALEVYKLTLEIKQLQLQIDKAKHVDREL

SEQ ID NO: 192 - protein, 57 aa, Synthetic construct
EYPEDCEQVHEGKKLMQCLPNLEEIKLALELYKLSLETKLLELQIDKEKKAKAKYSI

SEQ ID NO: 193 - protein, 57 aa, Synthetic construct
EYPEDCEQVHEGKKLMECLPTLEEIKLALALYKLSLETNLLELQIDKEKKAKAKYST

SEQ ID NO: 194 - protein, 57 aa, Synthetic construct
EIAEGCEQVLAGRKIMQCLPKPEDVRTALELYKLSLEIKQLEKKLEKEEKCTPEVQE

SEQ ID NO: 195 - protein, 67 aa, Synthetic construct
EYPEGCEQVVTGRKLLQCLSRPEEVKLALEVYKLSLEIEILQTNKLKKEAFLLREREKNVTCDFNPE

SEQ ID NO: 196 - protein, 52 aa, Synthetic construct
EYPEGCEQVVTGRKLLKCLSRPEEVKLALEVYKLSLEIALLELQIDKPKDAS

SEQ ID NO: 197 - protein, 52 aa, Synthetic construct
EVPENCEQVIVGKKLMKCLSNPDEAQMALQLYKLSLEAELLRLQIVKARQGS

SEQ ID NO: 198 - protein, 54 aa, Synthetic construct
EASEDLKPALTGNKTMQYVPNSHDVKMALEIYKLTLEVELLQLQIQKEKHTEAH

SEQ ID NO: 199 - protein, 51 aa, Synthetic construct
VSAEVCEAVFKGQKLLKCLPNAMEVKMALEVYKLSLEIEKLEQEKRKLEIA

SEQ ID NO: 200 - protein, 66 aa, Synthetic construct
EVPEECKQVAAGRKLLECLPNPSDVKMALEVYKLSLEIEQLEKEKYVKIQEKFSKKEMKQLTSALH

SEQ ID NO: 201 - protein, 59 aa, Synthetic construct
EVLEDCRIVSRGAQLLHCLSSPEDVHRALKVYKLFLEIERLEHQKEKWIQLHRKPQSMK

SEQ ID NO: 202 - protein, 63 aa, Synthetic construct
EGPEDCEIVNKGRQLLQCLSSPEDVQRALEVYKLSLEIERLEQQREKRTSVHRKAHYTKVDGP

SEQ ID NO: 203 - protein, 67 aa, Synthetic construct
EAPEGCEQVLTGRKLMQCLPSPEDVKVALEVYKLSLEIKQLEKERDKLMNTHQKFSEKEEMKDLFFP

SEQ ID NO: 204 - protein, 52 aa, Synthetic construct
EVPEGCEQVLTGKKLMQCLPNPEDVKMALEVYKLSLEIELLELQIDKARQGS

SEQ ID NO: 205 - protein, 47 aa, Synthetic construct
GCEQVLTGKRLMQCLPNPEDVKMALEVYKLSLEIEQLELQRDSARQS

SEQ ID NO: 206 - protein, 51 aa, Synthetic construct
ETPEGCEQVLTGKRLMQCLPNPEDVKMALEVYKLSLEIEQLELQRDSARQS

SEQ ID NO: 207 - protein, 53 aa, Synthetic construct
GSEQVLTGKRLMQSLPNPEDVKMALEVYKLSLEIEQLELQRDSARQSTLDKEL

SEQ ID NO: 208 - protein, 57 aa, Synthetic construct
ETPEGCEQVLTGKRLMQCLPNPEDVKMALEIYKLTLEIEQLELQRDSARQSTLDKEL

SEQ ID NO: 209 - protein, 57 aa, Synthetic construct
ETPEGCEQVLTGKRLMQCLPNPEDVKMALEIYKLSLEIKQLELQRDSARQSTLDKEL

SEQ ID NO: 210 - protein, 50 aa, Synthetic construct
EGCEQILTGKRLMQCLPDPEDVKMALEIYKLSLEIKQLELQRDRARQSTL

SEQ ID NO: 211 - protein, 57 aa, Synthetic construct
ETPEGCEQVLTGKRLMQCLPNPEDVKMALEVYKLSLEIKQLELQRDRARQSTLDKEL

SEQ ID NO: 212 - protein, 52 aa, Synthetic construct
EGCEQILTGKRLMQCLPNPEDVKMALEIYKLSLEIEQLELQRDRARQSTLDK

SEQ ID NO: 213 - protein, 61 aa, Synthetic construct
WETPEGCEQVLTGKRLMQCLPNPEDVKMALEVYKLSLEIEQLELQRDSARQSTLDKELVPR

SEQ ID NO: 214 - protein, 666 aa, Synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRGGGGSGGGGSGGGGSEVQLVESGGGLVQPGGSLRLSCAVSGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 215 - DNA, 1998 bp, Synthetic construct
  source 1..1998: mol_type = other DNA
gatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacggggtggtggtggctctgggggtggcggtagcggaggtggaggatcagaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttcagaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 216 - protein, 18 aa, Synthetic construct
GSTSGSGKPGSGEGSTKG

SEQ ID NO: 217 - DNA, 2595 bp, Synthetic construct
  source 1..2595: mol_type = other DNA
gatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacggggtggtggtggctctgggggtggcggtagcggaggtggaggatcagaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttcagaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcagcggcgccacaaacttctctctgctaaagcaagcaggtgatgttgaagaaaaccccgggcctatgtacagaatgcaactcctgtcttgtattgcactaagtctcgcacttgtcacaaacagttactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagac

SEQ ID NO: 218 - skipped

SEQ ID NO: 219 - DNA, 3732 bp, Synthetic construct
  source 1..3732: mol_type = other DNA
gatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacggggtggtggtggctctgggggtggcggtagcggaggtggaggatcagaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttcagaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcagcggcgccacaaacttctctctgctaaagcaagcaggtgatgttgaagaaaaccccgggcctatgtacagaatgcaactcctgtcttgtattgcactaagtctcgcacttgtcacaaacagttactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagacggctccggcgagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 220 - DNA, 10440 bp, Synthetic construct
  source 1..10440: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgccaccgccgccaccatgctgctgctggtgaccagcctgctgctgtgcgagctgccccaccccgcctttctgctgatccccggatccgggggtgggagtggtgggagtacgcgtgatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacggggtggtggtggctctgggggtggcggtagcggaggtggaggatcagaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttcagaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagttaacatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 221 - DNA, 11031 bp, Synthetic construct
  source 1..11031: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgccaccgccgccaccatgctgctgctggtgaccagcctgctgctgtgcgagctgccccaccccgcctttctgctgatccccggatccgggggtgggagtggtgggagtacgcgtgatatacaaatgactcagtctccttcttcactctcagcgagtgttggtgatcgagtaaccattacttgcaagtcaagccagtctttgttgtatagaagtaatcagaaaaactatctggcctggtaccaacagaagcccggtaaggcaccaaaattgctcatatactgggcatcaacacgggaaagtggtgtgccctcccgcttctctggcagtgggagtgggacagactttacgctgactatctcaagtcttcaaccggaagacttcgctacctactattgtcagcaatactataactatcctcggacctttggtcaaggtactaaggtcgagataaaacggggtggtggtggctctgggggtggcggtagcggaggtggaggatcagaagtgcaactcgtcgaaagcggtggcggacttgtccagccagggggatcactcagattgtcttgtgcagttagtggatactccataacaagtgattatgcctggaattgggtccgccaggctccgggaaaagggctcgaatgggtcgggtacatttctaactctggctctacatcctacaacccgagcttgaagtccaggttcactatatctagggataatagcaagaacactctgtacctccagatgaacagtctgagagcggaagatacagcagtgtattattgtgcgcgcgaaaggaactatgattatgacgactattattatgccatggattactggggtcagggcacccttgtgaccgtcagttcagaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggggcagcggcgccacaaacttctctctgctaaagcaagcaggtgatgttgaagaaaaccccgggcctatgtacagaatgcaactcctgtcttgtattgcactaagtctcgcacttgtcacaaacagttactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagacggctccggcgagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagttaacatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 222 - protein, 120 aa, Synthetic construct
ESKYGPPCPPCPKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 223 - protein, 114 aa, Synthetic construct
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0356935 A1
(43) Pub. Date: Nov. 12, 2026

(54) PRO-INFLAMMATORY CYTOKINE ENHANCED STEAP1 RECOMBINANT RECEPTORS

(71) Applicant: Fred Hutchinson Cancer Center, Seattle, WA (US)

(72) Inventor: John K. Lee, Seattle, WA (US)

(73) Assignee: Fred Hutchinson Cancer Center, Seattle, WA (US)

(21) Appl. No.: 19/484,811

(22) PCT Filed: May 16, 2024
(86) PCT No.: PCT/US2024/029755

§ 371 Date: Nov. 14, 2025

Related U.S. Application Data
(60) Provisional application No. 63/502,596, filed on May 16, 2023.

(51) Int. Cl. C07K 16/28; A61K 35/17; A61K 40/11; A61K 40/31; C07K 14/54; C07K 14/705; C07K 14/71; C12N 5/0783; C12N 9/06

(52) U.S. Cl. CPC C07K 16/28; A61K 35/17; A61K 40/11; A61K 40/31; C07K 14/54; C07K 14/7051; C07K 14/70521; C07K 14/70596; C07K 14/71; C12N 5/0636; C12N 9/003; A61K 2239/17; A61K 2239/21; A61K 2239/22; A61K 2239/25; A61K 2239/46; C07K 2317/524; C07K 2317/526; C07K 2317/53; C07K 2317/565; C07K 2317/622; C12N 2510/00

(57) ABSTRACT

Genetic constructs expressing a pro-inflammatory cytokine and a recombinant receptor, such as a chimeric antigen receptor (CAR), with a binding domain that binds STEAP1 are disclosed. The genetic constructs disclosed herein can be used in the treatment of prostate cancer, the Ewing family of tumors (EFT), bladder cancer, ovarian cancer, and rhabdomyosarcoma. The genetic constructs disclosed herein provide enhanced recombinant receptor cytotoxicity, the ability to bind and elicit cytotoxic effects even in low antigen density conditions, and enhanced interferon-gamma signaling which remodels the tumor microenvironment, further improving endogenous antitumor immunity.

### CROSS-REFERENCE TO RELATED APPLICATION

This application is a U.S. National Phase patent application based on International Patent Application No. PCT/US2024/029755, filed on May 16, 2025, which claims priority to U.S. Provisional Patent Application No. 63/502,596 filed May 16, 2023, each of which is incorporated herein by reference in its entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3IU9619.XML. The file is 258,048 bytes, was created on Nov. 13, 2025, and is being submitted electronically via Patent Center.

### FIELD OF THE DISCLOSURE

The current disclosure provides genetic constructs expressing pro-inflammatory cytokines and recombinant receptors, such as chimeric antigen receptors (CAR), with binding domains that bind STEAP1. The pro-inflammatory cytokine can include interleukin-18 (IL-18). The genetic constructs can provide enhanced recombinant receptor cytotoxicity and enhanced interferon-gamma signaling, improving antitumor immunity. The genetic constructs can be used in the treatment of STEAP1-expressing cancers, such as prostate cancer, the Ewing family of tumors (EFT), bladder cancer, ovarian cancer, and rhabdomyosarcoma.

### BACKGROUND OF THE DISCLOSURE

According to the World Health Organization, cancer is the second leading cause of death globally, and was responsible for nearly 10 million deaths in 2020.

Prostate cancer (PCa) is the second-leading cause of cancer death in men. The most common type of PCa is typically classified as an adenocarcinoma which is a type of cancer that forms in mucus-secreting glands of organs. PCa originates in the prostate gland (a gland that produces seminal fluid) of the male reproductive system. Localized prostate cancer (PCa) can be successfully treated, with nearly 100% survival at 5 years from diagnosis. Some methods of treatment for PCa involve surgery, radiation, cryotherapy or hormone therapy.

Hormone therapy can provide an effective therapy against PCa because its growth is often driven by male sex hormones called androgens, which include testosterone. In these hormone therapies, androgen levels in the man's body are reduced. Androgen levels can be lowered by surgically removing the testicles, by administering drugs that prevent the production of androgens, and/or by blocking the ability of androgens to have an effect in the body. Unfortunately, most hormone dependent cancers become refractory to these types of treatments after one to three years and resume growth despite hormone therapy. When hormone therapies lose their efficacy to treat PCa, the PCa is referred to as a “castration-resistant” prostate cancer or CRPC. Unfortunately, when PCa becomes CRPC, the 5-year survival rate drops to 30%. Thus, there remains a critical need for therapies to treat CRPC, among other low-survival cancers.

The Ewing family of tumors (EFT) is a family of small round blue cell tumors that arise from bone or soft tissue. This family represents the second most common malignant bone tumor in children and young adults, with an incidence of 200 cases per year in the United States.

For many years, the chosen treatments for cancer have been surgery, chemotherapy, and/or radiation therapy. In recent years, more targeted therapies have emerged to specifically target cancer cells by identifying and exploiting specific molecular and/or immunophenotypic changes seen primarily in those cells. For example, many cancer cells preferentially express particular markers on their cellular surfaces and these markers have provided targets for antibody-based therapeutics.

Significant progress has been made in genetically engineering cells of the immune system to target and kill unwanted cell types, such as cancer cells. Many of these immune cells are T cells that have been genetically engineered to express a recombinant receptor, for example, a chimeric antigen receptor (CAR). CAR are proteins with several distinct subcomponents that allow the genetically modified immune cells to recognize and kill targeted cell types. The subcomponents include at least an extracellular component and an intracellular component expressed as a single protein or in a form that assembles into a functional unit. The extracellular component includes a binding domain that specifically binds a marker (e.g., an antigen) that is preferentially present on the surface of unwanted cells such as cancerous cells. When the binding domain binds such markers, the intracellular component signals the immune cell to destroy the bound cell. STEAP1 is a protein with up-regulated expression in a number of cancers, such as prostate cancer, the Ewing family of tumors (EFT), bladder cancer, ovarian cancer, and rhabdomyosarcoma.

CAR can additionally include a transmembrane domain that can link the extracellular component to the intracellular component. Other subcomponents that can increase a CAR's function can also be used. For example, spacers provide CAR with additional conformational flexibility and often increase the binding domain's ability to bind the targeted antigen, leading to enhanced cytolytic effects. The appropriate length of a spacer within a particular CAR depends on numerous factors including how close or far a targeted marker is located from the surface of an unwanted cell's membrane. Thus, while the general structure of CAR are known, the ability of a particular CAR to elicit cytolytic effects in vivo remains an area of intense research and investigation. The ability of CAR to elicit cytolytic effects in low antigen density conditions, for example, remains a challenge. Moreover, CAR T cells can have suboptimal proliferation, functional anergy or exhaustion, impaired memory differentiation, and/or limited persistence limiting their ability to create or maintain remission in patients.

### SUMMARY OF THE DISCLOSURE

The current disclosure provides genetic constructs expressing a pro-inflammatory cytokine (e.g., interleukin-18 (IL-18)) and a recombinant receptor, such as a chimeric antigen receptor (CAR), with a binding domain that binds STEAP1. Constitutive expression of IL-18, for example, acts in cis by enhancing the cytotoxicity of a STEAP1 recombinant receptor and operates in trans by inducing interferon-gamma (IFN-γ) signaling to remodel the tumor microenvironment and enhance endogenous antitumor immunity. The STEAP1 recombinant receptor, itself, has additional benefits as it can bind and elicit cytotoxic effects even in low antigen density conditions and is highly specific against STEAP1. These features provide a significant clinical advance by allowing the use of recombinant receptor disclosed herein earlier in a cancer's progression before antigen density levels have risen due to growth and development of the cancer while at the same time minimizing off-target toxicities. Earlier intervention in a cancer's development often leads to significantly better treatment outcomes. Reduced potential toxicities are also of great clinical importance.

In particular embodiments, a genetic construct described herein includes a sequence encoding a pro-inflammatory cytokine. In particular embodiments, a genetic construct includes a sequence encoding a recombinant receptor including a STEAP1 binding domain. In particular embodiments, a genetic construct includes (i) a sequence encoding a pro-inflammatory cytokine, and (ii) a sequence encoding a recombinant receptor including a STEAP1 binding domain. In particular embodiments, the pro-inflammatory cytokine includes IL-18. In particular embodiments, the recombinant receptor includes a CAR. In particular embodiments, the STEAP1 binding domain includes an scFv binding domain derived from a vandortuzumab vedotin (DSTP3086S; a humanized variant of the murine monoclonal antibody mAb 120.545). In particular embodiments, the recombinant receptor further includes a transmembrane domain and an intracellular component. In particular embodiments, the intracellular component includes a CD3z activation domain and a 4-1BB costimulatory domain. In particular embodiments, the recombinant receptor further includes a long spacer including the IgG4 hinge-CH2-CH3 with a 4/2-NQ mutation in the CH2 domain. In particular embodiments, the transmembrane domain includes a CD28 transmembrane domain. In particular embodiments, the genetic construct further includes a sequence encoding a self-cleaving polypeptide. In particular embodiments, the self-cleaving polypeptide includes a 2A peptide from Thosea asigna virus (T2A). In particular embodiments, the self-cleaving polypeptide includes a 2A peptide from porcine teschovirus-1 (P2A).

In particular embodiments, the genetic construct is within a vector. In particular embodiments, the vector includes a lentiviral vector. In particular embodiments, the lentiviral vector includes a third-generation lentiviral vector. In particular embodiments, a genetic construct encoding a pro-inflammatory cytokine is within a first vector and a genetic construct encoding a recombinant receptor with a STEAP1 binding domain is within a second vector.

In particular embodiments, a genetic construct encoding a pro-inflammatory cytokine and a genetic construct encoding a recombinant receptor with a STEAP1 binding domain are expressed by the same cell. In particular embodiments, a genetic construct encoding a pro-inflammatory cytokine is expressed by a first cell and a genetic construct encoding a recombinant receptor including an STEAP1 binding domain is expressed by a second cell. In particular embodiments, a genetic construct encoding a pro-inflammatory cytokine and a genetic construct encoding a recombinant receptor including an STEAP1 binding domain are in the same vector.

The recombinant receptor disclosed herein can be used in the treatment of STEAP1-expressing cancers, such as prostate cancer, the Ewing family of tumors (EFT), bladder cancer, ovarian cancer, and rhabdomyosarcoma. In particular embodiments, the recombinant receptor disclosed herein can be used in the treatment of lethal, metastatic castration-resistant prostate cancer.

### BRIEF DESCRIPTION OF THE FIGURES

The patent or application file contains at least one drawing executed in color. Copies of this patent or patent application publication with color drawing(s) will be provided by the Office upon request and payment of the necessary fee. Applicant considers the color versions of the drawings as part of the original submission and reserves the right to present color images of the drawings in later proceedings.

FIGS. 1A-1E. Comparative analysis of six-transmembrane epithelial antigen of prostate 1 (STEAP1) and prostate-specific membrane antigen (PSMA) in lethal, metastatic castration-resistant prostate cancer (mCRPC). (1A) Characteristics of the mCRPC tissues represented on University of Washington Tissue Acquisition Necropsy Tissue Microarray 92 (UW TAN TMA92). (1B) Plot showing paired average H-scores of STEAP1 and PSMA immunohistochemical (IHC) staining from each mCRPC tissue core. From left to right, the mCRPC cores are: 10-013_BB, 10-013_Q, 10-013_R, 10-056_J, 10-056_K, 10-068_II, 10-068_PP, 11-028_DD, 11-028_H, 11-028_L, 12-005_H, 12-005_K, 12-005_QQ, 12-011_I, 12-011_J, 12-011_LL, 12-021_H, 12-021_I, 13-012_1, 13-012_L, 13-012_OO, 13-042_JJ, 13-042_M, 13-042_O, 13-084_H, 13-084_II, 13-099_H, 13-099_J, 13-101_K, 13-101_SS, 13-101_UU, 13-104_I, 13-104_K, 13-104_OO, 13-117_BB, 13-117_H, 13-117_OO, 13-122_H, 13-122_II, 13-122_OO, 14-031_GG, 14-031_H, 14-031_K, 14-039_E, 14-039_K, 14-043_CC, 14-043_H, 14-043_OO, 14-053_AA, 14-053_H, 14-053_O, 14-077_K, 14-077_N, 14-091_TT, 14-091_E, 14-096_FF, 14-096_II, 14-105_1, 14-105_J, 14-105_VV, 15-003_GG, 15-003_H, 15-010_H, 15-010_K, 15-019_CC, 15-019_HH, 15-023_I, 15-023_L, 15-023_LL, 15-069_1, 15-069_KK, 15-069_P, 15-090_H, 15-090_I, 15-090_LL, 15-096_FF, 15-096_H, 15-096_L, 16-052_BB, 16-052_JJ, 16-071_H, 16-071_1, 16-071_KK, 16-080_FF, 16-080_L, 16-080_LL, 16-101_EE, 16-101_J, 16-101_N, 17-007_AA, 17-007_EE, 17-007_H, 17-017_H, 17-017_O, 17-017_SS, 17-022_H, 17-022_JJ, 17-033_FF, 17-033_H, 17-036_H, 17-036_HH, 17-036_L, 17-042_GG, 17-042_H, 17-042_PP, 17-043_I, 17-043_II, 17-043_J, 17-057_J, 17-057_N, 17-072_E, 17-072_H, 17-081_M, and 17-081_P. (1C) Contingency table showing the frequency of mCRPC tissue cores with STEAP1 or PSMA IHC staining above or below an H-score threshold of 30. (1D, 1E) Photomicrographs of select mCRPC tissue cores after STEAP1 and PSMA IHC staining to highlight the (1D) absence of PSMA but presence of STEAP1 expression and (1E) intratumoral heterogeneity of PSMA expression but not STEAP1. Scale bars=50 μm.

FIGS. 2A-2D. Characteristics of STEAP1 expression in lethal mCRPC tissues. (2A) Photomicrographs of select mCRPC tissue cores after STEAP1 IHC staining to highlight the plasma membrane staining consistent with staining intensity scores of 0, 1, 2, and 3. (2B) Plot showing the STEAP1 H-scores of mCRPC tissues cores based on their metastatic site. Dashed line represents a STEAP1 H-score of 30. ** denotes p<0.01. Plots of STEAP1 H-score and (2C) Androgen receptor (AR) H-score or (2D) synaptophysin (SYP) H-score for each mCPC tissue core. Pearson correlation coefficient (r) and p values are shown.

FIGS. 3A-3I. Screening second-generation 4-1BB chimeric antigen receptors (CAR) to identify a lead for STEAP1 CAR T cell therapy. (3A) Schematic of the lentiviral STEAP1 CAR construct and variation based on short, medium, and long spacers. LTR=long terminal repeat; MNDU3=Moloney murine leukemia virus U3 region; scFv=single-chain variable fragment; VL=variable light chain; VH=variable heavy chain; tm=transmembrane; EGFRt=truncated epidermal growth factor receptor; 4/2 NQ=CH2 domain mutations to prevent binding to Fc-gamma receptors. (3B) Immunoblot analysis showing expression of STEAP1 in 22Rv1 parental cells, 22Rv1 STEAP1 knockout (ko) cells, and 22Rv1 STEAP1 ko cells with rescue of STEAP1 expression by lentiviral expression. GAPDH is used as a protein loading control. (3C) IFN-γ enzyme-linked immunosorbent assay (ELISA) results from co-cultures of either untransduced T cells or STEAP1-BBζ CAR T cells with each of the 22Rv1 sublines at a 1:1 ratio at 24 hours. n=4 replicates per condition. Bars represent Standard Deviation (SD). (3D) Relative cell viability of 22Rv1 target cells over time measured by fluorescence live cell imaging upon co-culture with (left) STEAP1-BBζ CAR T cells or (right) untransduced T cells at variable effector-to-target (E:T) cell ratios. (3E) Relative cell viability of 22Rv1 STEAP1 ko target cells over time measured by fluorescence live cell imaging upon co-culture with (left) STEAP1-BBζ CAR T cells or (right) untransduced T cells at variable E:T cell ratios. In FIG. 3D and FIG. 3E, n=4 replicates per condition and bars represent standard error or mean (SEM). (3F) Immunoblot analysis demonstrating expression of STEAP1 in androgen receptor (AR)-positive human prostate cancer cell lines but not AR-negative prostate cancer cell lines. GAPDH is used as a protein loading control. (3G) IFN-γ quantification by ELISA from co-cultures of either untransduced T cells or STEAP1-BBζ CAR T cells with each of the human prostate cancer cell lines in 3F at a 1:1 ratio at 24 hours. n=4 replicates per condition. Bars represent SD. (3H) Immunoblot analysis with prolonged exposure to evaluate expression of STEAP1 in 22Rv1, PC3, and PC3 STEAP1 ko sublines. GAPDH is used as a protein loading control. (3I) IFN-γ quantification by ELISA from co-cultures of either untransduced T cells or STEAP1-BBζ CAR T cells with each of the human prostate cancer cell lines in F at a 1:1 ratio at 24 hours. n=4 replicates per condition. Bars represent SD.

FIGS. 4A-4D. Validation of the antigen-specific activation and target cell cytolysis of STEAP1-BBζ CAR T cells. (4A) Representative flow cytometry plots showing immunophenotyping of CD4 and CD8 T cell products at day 10 of expansion from untransduced control and lentiviral STEAP1 CAR transduction conditions. IFN-γ quantification by ELISA from (4B) control culture conditions or (4C) co-cultures of either untransduced T cells or STEAP1-BBζ CAR T cells with the DU145 or DU145 STEAP1 cell lines at a 1:1 ratio at 24 hours. n=4 replicates per condition. Bars represent SD. (4D) Relative cell viability of DU145 STEAP1 target cells over time measured by fluorescence live cell imaging upon co-culture with STEAP1-BBζ CAR T cells or untransduced T cells at a 1:1 ratio. n=4 replicates per condition. Bars represent SEM.

FIGS. 5A-5G. Determination of the STEAP1 ectodomain specificity of STEAP1-BBζ CAR T cells using mouse/human Steap1 chimeras. (5A) Immunoblot analysis confirming the lack of human STEAP1 (hSTEAP1) and mouse Steap1 (mSteap1) expression in the DU145 cell line and their respective expression in the lentivirally engineered DU145 hSTEAP1 and DU145 mSteap1 lines. GAPDH is used as a protein loading control. (5B) IFN-γ quantification by ELISA from (5B) co-cultures of STEAP1-BBζ CAR T cells with the DU145, DU145 hSTEAP1, and DU145 mSteap1 cell lines at a 1:1: ratio or (5C) control culture conditions at 24 hours. n=4 replicates per condition. Bars represent SD. (5D) Schematic of the mSteap1 protein with extracellular domains (mECDs, boxes without triangle) highlighted and the mouse/human Steap1 chimeric proteins each with individual replacement of a mECD with the counterpart hSTEAP1 extracellular domain (hECD, box with triangle). (5E) IFN-γ quantification by ELISA co-cultures of STEAP1-BBζ CAR T cells with each of the DU145 lines engineered to express hSTEAP1, mSteap1, or mouse/human Steap1 chimeric proteins at a 1:1 ratio at 24 hours. n=4 replicates per condition. Bars represent SD. (5F) Relative cell viability of each of the targets in FIG. 5E over time measured by fluorescence live cell imaging upon co-culture with STEAP1-BBζ CAR T cells at a 1:1 ratio. n=4 replicates per condition. Bars represent SEM. (5G) Alignment of the amino acid sequences of the hSTEAP1 ECD2 (SEQ ID NO: 68) and mSteap1 ECD2 (SEQ ID NO: 69) showing non-conserved residues at positions 198 and 209.

FIGS. 6A-6D. Evaluation of the reactivity of STEAP1-BBζ CAR T cells to STEAP1B isoforms. (6A) Alignment of the amino acid sequence of the hSTEAP1 ECD2 (SEQ ID NO: 68) to human STEAP1B isoforms 1 (SEQ ID NO: 68), 2 (SEQ ID NO: 68), and 3 (SEQ ID NO: 68) showing complete sequence conservation. (6B) Plots of TOPCONS consensus predictions of membrane protein topology and reliability scores for hSTEAP1, mSteap1, and the three human STEAP1B isoforms. (6C) Plots of membrane protein topology predictions for human STEAP1B isoform 1 showing disagreement between different algorithms. (6D) IFN-γ quantification by ELISA co-cultures of STEAP1-BBζ CAR T cells with each of the DU145 lines engineered to express hSTEAP1, mSteap1, or human STEAP1B isoforms at a 1:1 ratio at 24 hours. n=4 replicates per condition. Bars represent standard deviation. This data shows a lack of reactivity to STEAP1B isoforms, highlighting the specificity of the disclosed CAR, greatly minimizing or even eliminating the potential for off-target toxicities.

FIGS. 7A-7H. In vivo antitumor activity of STEAP1-BBζ CAR T cell therapy in prostate cancer models with native STEAP1 expression. (7A) Volumes of 22Rv1 subcutaneous tumors in NSG mice over time after a single intratumoral injection of 5×10⁶ untransduced T cells or STEAP1-BBζ CAR T cells at normal CD4/CD8 ratios. Bars represent SD. * denotes p<0.05; **** denotes p<0.0001. (7B) Schematic of tumor challenge experiments for 22Rv1 (top) and C4-2B (bottom) disseminated models. fLuc=firefly luciferase; BLI=bioluminescence imaging. (7C) Serial live BLI of NSG mice engrafted with 22Rv1-fLuc metastases and treated with a single intravenous injection of 5×10⁶ untransduced T cells or STEAP1-BBζ CAR T cells at normal CD4/CD8 ratios on day 0. The X denotes deceased mice. Radiance scale is shown. (7D) Plot showing the quantification of total flux over time from live BLI of each mouse in FIG. 7C. (7E) Kaplan-Meier survival curves of mice in FIG. 7C with statistical significance determined by log-rank test. (7F) Serial live BLI of NSG mice engrafted with C4-2B metastases and treated with a single intravenous injection of 5×10⁶ untransduced T cells or STEAP1-BBζ CAR T cells at normal (i.e., 1:1) CD4/CD8 ratios on day 0. The X denotes deceased mice. Radiance scale is shown. (7G) Plot showing the quantification of total flux over time from live BLI of each mouse in FIG. 7F. (7H) Quantification of CD3+EGFRt+ STEAP1-BBζ CAR T cells by flow cytometry from splenocytes of mice treated with STEAP1-BBζ CAR T cells at the end of experiment on day 49. All experiments shown were performed at least twice.

FIGS. 8A-8D. Therapeutic antitumor activity of STEAP1-BBζ CAR T cell therapy in human prostate cancer cell line xenograft models. (8A) Photomicrograph of CD3 IHC staining of a 22Rv1 subcutaneous tumor 25 days after intratumoral treatment with STEAP1-BBζ CAR T cells. Scale bars=100 μm. (8B) Plot of average weights of NSG mice engrafted with 22Rv1-fLuc metastatic tumors over time after treatment with either untransduced T cells or STEAP1-BBζ CAR T cells. Bars represent SD. (8C) Plot of average weights of NSG mice engrafted with C4-2B-fLuc metastatic tumors over time after treatment with either untransduced T cells or STEAP1-BBζ CAR T cells. Bars represent SD. (8D) Ex vivo BLI of liver and lung tissues from NSG mice engrafted with C4-2B-fLuc metastatic tumors and treated with either (left) untransduced T cells or (right) STEAP1-BBζ CAR T cells. Radiance scale is shown.

FIGS. 9A-9H. Establishing a mouse-in-mouse system with a novel human STEAP1 knock-in (hSTEAP1-KI) mouse model and murinized STEAP1 CAR. (9A) Schematic showing the homologous recombination strategy using a targeting vector to knock-in human STEAP1 exons 2-5 into the mouse Steap1 locus on the C57Bl/6 background. FRT=Flippase recognition target. (9B) Visualization of PCR products from tail tip genotyping of wildtype (+/+), heterozygous (KI/+), or homozygous (KI/KI) mice using primer pairs intended to amplify portions of wildtype or hSTEAP1-KI alleles. NTC=null template control. (9C) qPCR for human STEAP1 expression normalized to 18S expression in a survey of tissues from hSTEAP1-KI/+ mice. n=3 for sex-specific organs and n=6 for common organs. Bars represent SD. Photomicrographs of STEAP1 IHC staining of (9D) prostate tissues from (left) +/+ and (right) KI/+ mice and (9E) an adrenal gland from a KI/+ mouse. Scale bars=50 μm. (9F) Schematic of the retroviral murinized STEAP1 CAR construct. MuLV=murine leukemia virus; mCD19t=mouse truncated CD19. (9G) Quantification of the efficiency of retroviral transduction of activated mouse T cells from three independent experiments based the frequency of mouse CD3+CD19t+ cells by flow cytometry. (9H) Relative cell viability of RM9 or RM9-hSTEAP1 target cells over time measured by fluorescence live cell imaging upon co-culture at a 1:1 ratio with mouse STEAP1-mBBζ CAR T cells or untransduced T cells. n=4 replicates per condition. Bars represent SEM. *** denotes p<0.001.

FIGS. 10A-10H. Determination of the efficacy and safety of mouse STEAP1-mBBζ CAR T cells in hSTEAP1-KI mice bearing syngeneic, disseminated prostate cancer. (10A) Schematic of the tumor challenge experiment for the RM9-hSTEAP1 disseminated model in hSTEAP1-KI/+ mice. Cy=cyclophosphamide (for preconditioning). (10B) Serial live BLI of hSTEAP1-KI/+ mice engrafted with RM9-hSTEAP1-fLuc metastases and treated with a single intravenous injection of 5×10⁶ mouse untransduced T cells or STEAP1-mBBζ CAR T cells on day 0. The X denotes deceased mice. Radiance scale is shown. (10C) Plot showing the quantification of total flux over time from live BLI of each mouse in FIG. 10B. (10D) Kaplan-Meier survival curves of mice in FIG. 10B with statistical significance determined by log-rank test. Plots of weights for each mouse (numbered in FIG. 10B) over time in the (10E) mouse untransduced T cell treatment group and (10F) STEAP1-mBBζ CAR T cell treatment group. (10G) Photomicrographs of STEAP1 IHC staining of RM9-hSTEAP1 tumors after treatment with mouse untransduced T cells showing regions of (left) strong homogenous STEAP1 expression and (right) heterogeneous STEAP1 expression. Scale bars=50 μm. (10H) Representative STEAP1 IHC staining of RM9-hSTEAP1 tumors after treatment with STEAP1-mBBζ CAR T cells that demonstrate no STEAP1 expression. Scale bars=50 μm.

FIGS. 11A, 11B. Preserved tissue architecture and absence of increased T cell infiltration in the prostates or adrenal glands of hSTEAP1-KI/+ mice treated with mouse STEAP1-mBBζ CAR T cells. Representative photomicrographs of hematoxylin & eosin (H&E) and CD3 IHC staining of hSTEAP1-KI/+ prostates from mice treated with (11A) untransduced T cells and (11B) STEAP1-mBBζ CAR T cells. Arrowheads indicate rare CD3+ cells. Scale bars=50 μm.

FIGS. 12A-12G. (12A) IFN-γ enzyme-linked immunosorbent assay (ELISA) results from co-cultures of either untransduced T cells or STEAP1 short spacer CAR T cells or STEAP1 medium spacer CAR T cells with each of the 22Rv1 sublines at a 1:1 ratio at 24 hours. n=4 replicates per condition. Bars represent SD. (12B) Plot and table showing absolute quantitation of STEAP1 molecules per cell in the 22Rv1, C4-2B, PC3, and DU145 prostate cancer cell lines as determined by flow cytometry using Bangs Laboratories Quantum Simply Cellular Microspheres. (12C) Relative cell viability of PC3 target cells over time measured by fluorescence live cell imaging upon co-culture with untransduced T cells or STEAP1-BBζ CAR T cells at a 1:1 ratio. n=4 replicates per condition and bars represent SEM. (12D) Schematic of tumor challenge experiments for the PC3 disseminated model. (12E) Serial live bioluminescence imaging (BLI) of NSG mice engrafted with PC3-fLuc metastases and treated with a single intravenous injection of 5×10⁶ untransduced T cells or STEAP1-BBζ CAR T cells at a normal CD4/CD8 ratio on day 0. Mice were euthanized due to the onset of severe acute graft-versus-host (GVHD) in both treatment arms in week 4. Radiance scale is shown. (12F) Plot showing the quantification of total flux over time from live BLI of each mouse in FIG. 12E. (12G) Relative cell viability of Ewing sarcoma RD-ES and SK-ES-1 target cells over time measured by fluorescence live cell imaging upon co-culture with untransduced T cells or STEAP1-BBζ CAR T cells at a 1:1 ratio. n=4 replicates per condition and bars represent SEM.

FIGS. 13A, 13B. STEAP1-BBζ CAR T therapy is active in vivo against preclinical orthotopic models of Ewing Sarcoma for (13A) SK-ES-1 model and (13B) RD-ES model.

FIG. 14. IL-18 secretion enhances antigen-specific killing of target cells. Plots showing relative cell viabilities of target cells (STEAP1-RM9-CGW or STEAP1⁺ RM9-hSTEAP1) over time upon co-culture with T cells (untransduced, STEAP1 CAR, IL-18, or IL-18 STEAP1 CAR) at variable effector:target (1:1, 1:2.5, 1:5) ratios. Enhanced antigen-specific cytotoxicity of IL-18 armored STEAP1 CAR T cells relative to STEAP1 CAR T cells is evident at lower effector:target ratios.

FIGS. 15A, 15B. IL-18 secretion from STEAP1 CAR T cells extends survival of mice bearing disseminated RM9-hSTEAP1 tumors. C57Bl/6 mice were inoculated with syngeneic RM9-hSTEAP1-fLuc cells by tail vein injection on day −7 and received 5×10⁶ untransduced, STEAP1 CAR, or IL-18 STEAP1 CAR T cells by tail vein injection on day 0. (15A) Live bioluminescence imaging was performed twice weekly after treatment to monitor tumor burden. (15B) Kaplan-Meier survival curves showing extension of survival associated with IL-18 armored STEAP1 CAR T cell therapy.

FIGS. 16A, 16B. STEAP1 CAR T cell therapy is active against a preclinical disseminated model of Ewing sarcoma and demonstrates dose-dependence. NSG mice were inoculated with SK-ES-1-fLuc cells by tail vein injection on day −14 and received 5×10⁶ untransduced T cells or STEAP1 CAR T cells, 1×10⁶ STEAP1 CAR T cells, or 2×10⁵ STEAP1 CAR T cells. (16A) Live bioluminescence imaging was performed twice weekly after treatment to monitor tumor burden. (16B) Kaplan-Meier survival curves showing a dose-dependence of STEAP1 CAR T cell therapy on the survival of mice bearing disseminated SK-ES-1 tumors.

FIGS. 17A-17C. IL-18 armoring of STEAP1 CAR T cells enhances in vivo therapeutic efficacy. NSG mice were inoculated with SK-ES-1-fLuc cells by tail vein injection on day −14 and received 1×10⁶ untransduced T cells, 1×10⁶ STEAP1 CAR T cells or IL-18 STEAP1 CAR (STEAP1-18) T cells, or 2×10⁵ STEAP1 CAR T cells or IL18 STEAP1 CAR (STEAP1-18) T cells by tail vein injection on day 0. (17A) Schematic of STEAP1 CAR construct. (17B) Live bioluminescence imaging was performed after treatment to monitor tumor burden. (17C) Kaplan-Meier survival curves showing enhanced survival of mice bearing disseminated SK-ES-1 tumors treated with IL-18 armored STEAP1 CAR T cells relative to STEAP1 CAR T cells and untransduced T cells at a dose of 1×10⁶ cells.

FIG. 18. Sequences supporting the disclosure including STEAP1_VL coding sequence (SEQ ID NO: 70); (G4S)3 Linker coding sequence (SEQ ID NO: 71); STEAP1_VH coding sequence (SEQ ID NO: 72); STEAP1 scFv (VL-VH) (SEQ ID NO: 73); STEAP1_scFv coding sequence (SEQ ID NOs: 74 and 76); STEAP1 scFv (VH-VL) (SEQ ID NO: 75); IgG4 hinge region (SEQ ID NO: 77) and coding sequences (SEQ ID NOs: 78-80); IgG4-CH2 domain (SEQ ID NO: 81) and coding sequence (SEQ ID NO: 82); IgG4-int (DS) coding sequence (SEQ ID NO: 83); IgG4 CH3 domain (SEQ ID NO: 84) and coding sequence (SEQ ID NO: 85); Intermediate Spacer (SEQ ID NO: 222) and coding sequence (SEQ ID NO: 86); Long Spacer (SEQ ID NO: 87) and coding sequence (SEQ ID NO: 88); CD3ζ signaling domain (SEQ ID NOs: 89, 90, and 223) and coding sequences (SEQ ID NOs: 91 and 92); 4-1BB costimulatory domain (SEQ ID NOs: 93-95) and coding sequences (SEQ ID NOs: 96-98); CD28 Transmembrane Domain (SEQ ID NOs: 99-101) and coding sequences (SEQ ID NOs: 102-105); EGFRt (SEQ ID NO: 106) and coding sequence (SEQ ID NO: 107); tCD19 coding sequence (SEQ ID NO: 108); Thosea asigna Virus 2A (T2A) Peptide (SEQ ID NOs: 109-111) and coding sequenced (SEQ ID NOs: 112-114); Porcine Teschovirus-1 2a (P2A) Peptide (SEQ ID NOs: 115 and 116) and coding sequence (SEQ ID NO: 117); Equine Rhinitis A Virus (ERAV) 2A (E2A) Peptide (SEQ ID NO: 118); Foot-And-Mouth Disease Virus 2A (F2A) Peptide (SEQ ID NO: 119); IL2 signal sequence (SEQ ID NO: 120) and coding sequence (SEQ ID NO: 121); CMV promoter sequence (SEQ ID NO: 122); EF1 promoter coding sequence-A (SEQ ID NOs: 123 and 124); Psi coding sequence (SEQ ID NOS: 125 and 126); RRE coding sequence (SEQ ID NOs: 127 and 128); Flap (SEQ ID NO: 129); GM-CSF signal peptide coding sequence (SEQ ID NO: 130); GM-CSFR coding sequence (SEQ ID NO: 131); dWPRE coding sequence (SEQ ID NO: 132); WPRE (SEQ ID NO: 133); U3 coding sequence (SEQ ID NO: 134); delU3 (SEQ ID NO: 135); R coding sequence (SEQ ID NOs: 136 and 137); U5 coding sequence (SEQ ID NOs: 138 and 139); AmpR coding sequence (SEQ ID NO: 140); AmpR (SEQ ID NO: 141); ColE1 origin coding sequence (SEQ ID NOs: 142 and 143); LacO coding sequence (SEQ ID NO: 144); M13-rev coding sequence (SEQ ID NO: 145); T3 coding sequence (SEQ ID NO: 146); cPPT coding sequence (SEQ ID NO: 147); MNDU3 promoter coding sequence (SEQ ID NO: 148); EcoR1 coding sequence; Kozak coding sequence; T7 coding sequence (SEQ ID NO: 149); M13-fwd coding sequence (SEQ ID NO: 150); LacZ alpha coding sequence (SEQ ID NO: 151); M13 origin coding sequence (SEQ ID NO: 152); SV40 (SEQ ID NO: 153); 6-histidine tag coding sequence (SEQ ID NO: 154); 3× glycine linker and coding sequence; Portion of CD22 extracellular domain (SEQ ID NOs: 155-158); PRKAR1A dimerization domain (SEQ ID NO: 159 and 160); PRKAR1B dimerization domain (SEQ ID NO: 161); PRKAR1R dimerization domain (SEQ ID NO: 162 and 163); PRKAR1E dimerization domain (SEQ ID NO: 164); Dimerization and Docking Domain (DDD) (SEQ ID NOs: 165 and 166); Anchoring Domain (AD) (SEQ ID NOs: 167 and 168); Jun Leucine Zipper (SEQ ID NO: 169); Fos Leucine Zipper (SEQ ID NO: 170); consensus sequence for a WW motif (SEQ ID NO: 171); SH2B adapter protein dimerization domain (SEQ ID NO: 172); SH3 domain that allows homodimerization of IB1 (SEQ ID NO: 173); PTB domain of human DOK-7 (SEQ ID NO: 174); PDZ-like domain of SATB1 (SEQ ID NO: 175); WD40 repeats of APAF (SEQ ID NO: 176); PAS motif of the dioxin receptor (SEQ ID NO: 177); EF hand motif of parvalbumin (SEQ ID NO: 178); FKBP12 domain (SEQ ID NO: 179); FRB segment of mTOR (SEQ ID NO: 180); C4b Multimerization Domains (SEQ ID NOs: 181-213); STEAP1-BBζ CAR (SEQ ID NO: 214) and coding sequence (SEQ ID NO: 215); CAR and hIL18 coding sequence (SEQ ID NO: 217); CAR and hIL18 and EGFRt coding sequence (SEQ ID NO: 219); pCCLc-MNDU3-STEAP1 binding domain-IgG4Hinge-CH2-CH3-4-2NQ-CD28TM-41BBSig-CD3zeta-T2A-EGFRt (SEQ ID NO: 220); AND STEAP1CAR_IL-2SP_hIL-18_EGFRt coding sequence (SEQ ID NO: 221).

### DETAILED DESCRIPTION

According to the World Health Organization, cancer is the second leading cause of death globally, and was responsible for nearly 10 million deaths in 2020. Prostate cancer (PCa) is the most frequently diagnosed cancer in men aside from skin cancer and is the second-leading cause of cancer death in men. The most common type of PCa is typically classified as an adenocarcinoma which is a type of cancer that forms in mucus-secreting glands of organs. PCa originates in the prostate gland (a gland that produces seminal fluid) of the male reproductive system. Localized PCa can be successfully treated, with nearly 100% survival at 5 years from diagnosis. Some methods of treatment for PCa involve surgery, radiation, cryotherapy or hormone therapy.

Hormone therapy can provide an effective therapy against PCa because its growth is often driven by male sex hormones called androgens, which include testosterone. In these hormone therapies, androgen levels in the man's body are reduced. Androgen levels can be lowered by surgically removing the testicles, by administering drugs that prevent the production of androgens, and/or by blocking the ability of androgens to have an effect in the body. Unfortunately, most hormone dependent cancers become refractory to these types of treatments after one to three years and resume growth despite hormone therapy. When hormone therapies lose their efficacy to treat PCa, the PCa is referred to as a “castration-resistant” prostate cancer or CRPC. Unfortunately, when PCa becomes CRPC, the 5-year survival rate drops to 30%. Thus, there remains a critical need for therapies to treat CRPC, among other low-survival cancers.

The Ewing family of tumors (EFT) is a family of small round blue cell tumors that arise from bone or soft tissue. This family represents the second most common malignant bone tumor in children and young adults, with an incidence of 200 cases per year in the United States. Esiashvili et al., J Pediatr Hematol Oncol. 30 (6): 425-30 (2008). EFT is characterized by a specific translocation involving the EWS (Ewing's sarcoma gene) on chromosome 22 with one of the E26 transformation-specific transcription factory family genes.

For many years, the chosen treatments for cancer have been surgery, chemotherapy, and/or radiation therapy. In recent years, more targeted therapies have emerged to specifically target cancer cells by identifying and exploiting specific molecular changes seen primarily in those cells. For example, many cancer cells preferentially express particular markers (e.g., antigens) on their cellular surface and these markers have provided targets for antibody-based therapeutics.

Significant progress has been made in genetically engineering cells of the immune system to target and kill unwanted cell types, such as cancer cells. Many of these immune cells are T cells that have been genetically engineered to express a recombinant receptor, such as a chimeric antigen receptor (CAR). CAR are proteins with several distinct subcomponents that allow the genetically modified immune cells to recognize and kill targeted cell types such as cancerous cells. The subcomponents include at least an extracellular component and an intracellular component, when expressed by a cell. The extracellular component includes a binding domain that specifically binds a marker (e.g., antigen) that is preferentially present on the surface of unwanted cells such as cancerous cells. The binding domain is typically a single-chain variable fragment (scFv) derived from a monoclonal antibody (mAb), but it can be based on other formats which specifically bind the marker of interest.

When the binding domain binds such a marker, the intracellular component signals the immune cell to destroy the bound cell. The intracellular components provide such activation signals based on the inclusion of an effector domain. First generation CAR utilize the cytoplasmic domain of CD3ζ as an effector domain. Second generation CAR utilize the cytoplasmic domain of CD3ζ in combination with cluster of differentiation 28 (CD28) or 4-1BB (CD137) cytoplasmic domains, while third generation CAR utilize the CD3ζ cytoplasmic domain in combination with the CD28 and 4-1BB cytoplasmic domains as effector domains.

CAR can additionally include a transmembrane domain that links the extracellular component to the intracellular component, however not all CAR require transmembrane domains.

Other subcomponents of CAR can be used to increase a CAR's function. For example, spacers provide CAR with additional conformational flexibility and often increase the binding domain's ability to bind the targeted antigen. The appropriate length of a spacer within a particular CAR depends on numerous factors including how close or far a targeted marker is located from the surface of an unwanted cell's membrane.

A key to successful targeted therapy is in the choice of the target antigen (or target marker). An ideal target marker can be immunogenic, play a critical role in proliferation and/or differentiation, is expressed only on the surface of all malignant cells and malignant stem cells, and/or at least a large portion of patients should test positive for the target marker (Cheever, et al., 2009. Clin. Cancer Res. 15(17): 5323-8337).

STEAP1 (also known as PRSS24, STEAP, six transmembrane epithelial antigen of the prostate 1, or STEAP family member 1), is a 339-amino-acid protein named for its 6 transmembrane spanning regions. Transcriptome and proteome analyses as well as functional studies show that STEAP1 expression correlates with oxidative stress responses and elevated levels of reactive oxygen species.

Further, STEAP1 is upregulated in a variety of tumors, including prostate, bladder, ovarian, rhabdomyosarcoma, and EFT (Hubert et al., Proc Natl Acad Sci USA 96 (25): 14523-8 (1999); Rodeberg et al., Clin Cancer Res 11 (12): 4545-52 (2005)). In prostate cancer specifically, STEAP1 is expressed in up to 88% of lethal, metastatic disease whereas prostate-specific membrane antigen (PSMA) is expressed in up to 61% of lethal, metastatic disease. STEAP1 also can serve as an immunohistological marker for patients with EFT; 71 of 114 (62.3%) EFT samples displayed detectable membranous STEAP1 immunoreactivity (Grunewald et al., Ann Oncol, 23 (8): p. 2185-90 (2012)). Another genetic profiling study done in EFT patients showed that the absence of STEAP1 transcript in the bone marrow was strongly correlated with patient overall survival and survival without new metastases. Given the expression of STEAP1 in more than 60% of EFT tumors but with limited expression in normal tissue (secretory tissue of the bladder and prostate), STEAP1 can serve as a useful target for antibody-based and immune-cell based strategies.

Human STEAP1 (NCBI Reference Sequence: NP 036581.1) has the following amino
acid sequence:
(SEQ ID NO: 1)
MESRKDITNQEELWKMKPRRNLEEDDYLHKDTGETSMLKRPVLLHLHQTAHADEFDCPSELQ
HTQELFPQWHLPIKIAAIIASLTFLYTLLREVIHPLATSHQQYFYKIPILVINKVLPMVSITLLALVYLP
GVIAAIVQLHNGTKYKKFPHWLDKWMLTRKQFGLLSFFFAVLHAIYSLSYPMRRSYRYKLLNWA
YQQVQQNKEDAWIEHDVWRMEIYVSLGIVGLAILALLAVTSIPSVSDSLTWREFHYIQSKLGIVS
LLLGTIHALIFAWNKWIDIKQFVWYTPPTFMIAVFLPIVVLIFKSILFLPCLRKKILKIRHGWEDVTKI
NKTEICSQL.
Mouse STEAP1 (NCBI Reference Sequence: NP 081675.2) has the following amino
acid sequence:
(SEQ ID NO: 2)
MEISDDVTNPEQLWKMKPKGNLEDDSYSTKDSGETSMLKRPGLSHLQHAVHVDAFDCPSELQ
HTQEFFPNWRLPVKVAAIISSLTFLYTLLREIIYPLVTSREQYFYKIPILVINKVLPMVAITLLALVYL
PGELAAVVQLRNGTKYKKFPPWLDRWMLARKQFGLLSFFFAVLHAVYSLSYPMRRSYRYKLL
NWAYKQVQQNKEDAWVEHDVWRMEIYVSLGIVGLAILALLAVTSIPSVSDSLTWREFHYIQSKL
GIVSLLLGTVHALVFAWNKWVDVSQFVWYMPPTFMIAVFLPTLVLICKIALCLPCLRKKILKIRCG
WEDVSKINRTEMASRL.
Canine STEAP1 (NCBI Reference Sequence: XP 013974694.1) has the following
amino acid sequence:
(SEQ ID NO: 3)
MESRQDITSQEELWTMKPRRNLEEDDYLDKDSGDTRVLKRPVLLHMHQTTHFDEFDCPAELK
HKQELFPMWRWPVKIAAVISSLTFLYTLLREIIHPFVTSHQQYFYKIPILVINKVLPMVSITLLALVY
LPGVIAAVVQLHNGTKYKKFPHWLDRWMLTRKQFGLLSFFFAVLHAIYSLSYPMRRSYRYKLLN
WAYQQVQQNKEDAWIEHDVWRMEIYVSLGIVTLAILALLAVTSIPSVSDSLTWREFHYIQSKLG
MVSLLLGTIHALIFAWNKWVDIKQFVWYTPPTFMIAVFLPIVVLICKAILFLPCLRKKILKIRHGWE
DVTKINKTEMS.

While recombinant receptors have provided significant anti-cancer effects in some situations, the ability of a particular recombinant receptor to consistently elicit cytolytic effects in vivo remains an area of intense research and investigation. The ability of a recombinant receptor to elicit cytolytic effects in low antigen density conditions, for example, remains a challenge. Moreover, immune cells engineered to express a recombinant receptor can have suboptimal proliferation, functional anergy or exhaustion, impaired memory differentiation, and/or limited persistence limiting their ability to create or maintain remission in patients.

The effects of an immune cell therapy, such as CAR T cell therapy, can be boosted by administering cytokines that promote the immune response. Cytokines affect the growth of blood cells and other cells involved in the immune and inflammation response. In this way, various cytokines can help boost anti-cancer activity. More specifically, pro-inflammatory cytokines activate immune cells and induce the production of inflammatory proteins to inhibit the growth of tumors. For example, interleukin (IL)-18 is a pro-inflammatory cytokine that plays an anti-cancer role by promoting a type 1 immune response.

The current disclosure provides genetic constructs expressing a pro-inflammatory cytokine (e.g., IL-18) and a recombinant receptor for the treatment of STEAP1-related disorders, such as prostate cancer, EFT, bladder cancer, ovarian cancer, and rhabdomyosarcoma. In particular embodiments, the genetic constructs disclosed herein can be used in the treatment of lethal, metastatic castration-resistant prostate cancer. In particular embodiments, the genetic constructs disclosed herein can be used in the treatment of EFT. In particular embodiments, the pro-inflammatory cytokine includes IL-18, IL1a, IL-1B, IL-33, IL-36a, IL-36B, or IL-36γ. In particular embodiments, constitutive expression of the pro-inflammatory cytokine by genetic constructs described herein enhances the cytotoxicity of the STEAP1 recombinant receptor and induces interferon-gamma (IFN-γ) signaling to remodel the tumor microenvironment and enhance endogenous antitumor immunity. In particular embodiments, the recombinant receptor expressed by genetic constructs described herein provide cytolytic activity even in low antigen density conditions and shows little to no cross reactivity with the highly related STEAP1B. For example, data presented herein shows significant cytolytic activity against the PC3 cell line expressing 1,491 STEAP1 molecules per cell. This is in comparison to the 22Rv1 cell line and the C4-2B cell line, which express 69,475 STEAP1 molecules per cell or 67,462 STEAP1 molecules per cell, respectively. Furthermore, data presented herein shows that although the recombinant receptor (e.g., CAR) described herein is highly reactive against STEAP1, it is not reactive against STEAP1B isoforms which are proteins with the greatest homology to STEAP1. This shows that not only do the recombinant receptors described herein provide cytolytic activity even in low antigen density conditions, but that they are also highly specific for STEAP1, reducing clinical concern regarding off-target reactivity. The ability to provide cytolytic activity in such low antigen density conditions provides an important clinical benefit as treatment can be effectively administered before tumor burden expands to generate high antigen density conditions.

As used herein, “low antigen density conditions” refer to a cancer antigen expression level of less than 50,000 antigen molecules per diseased cell, less than 40,000 antigen molecules per diseased cell, less than 30,000 antigen molecules per diseased cell, less than 20,000 antigen molecules per diseased cell, less than 10,000 antigen molecules per diseased cell, less than 5,000 antigen molecules per diseased cell, less than 4,000 antigen molecules per diseased cell, less than 3,000 antigen molecules per diseased cell, less than 2,000 antigen molecules per diseased cell, or less than 1,500 antigen molecules per diseased cell.

As used herein, “low STEAP1 antigen density conditions” refer to a STEAP1 expression level of less than 50,000 STEAP1 molecules per diseased cell, less than 40,000 STEAP1 molecules per diseased cell, less than 30,000 STEAP1 molecules per diseased cell, less than 20,000 STEAP1 molecules per diseased cell, less than 10,000 STEAP1 molecules per diseased cell, less than 5,000 STEAP1 molecules per diseased cell, less than 4,000 STEAP1 molecules per diseased cell, less than 3,000 STEAP1 molecules per diseased cell, less than 2,000 STEAP1 molecules per diseased cell, or less than 1,500 STEAP1 molecules per diseased cell.

A “STEAP1-related disorder” is one where diseased or infected cells within a subject express STEAP1, such that STEAP1 provides an antigen for the targeted delivery of therapeutic treatments. In these disorders, STEAP1 should be preferentially-expressed by the diseased or infected cells such that on-target/off-site side effects are minimized or eliminated.

Diseased cells expressing STEAP1 are cells targeted for destruction by a treatment described herein. Diseased cells expressing STEAP1 include, for example, prostate cancer cells (e.g., castration-resistant prostate cancer cells), the Ewing family of tumor cells (including Ewing's sarcoma cells), bladder cancer cells, breast cancer cells, ovarian cancer cells, colon cancer cells, lung cancer cells, and kidney cancer cells.

In particular embodiments, a genetic construct includes a sequence encoding a pro-inflammatory cytokine. In particular embodiments, a genetic construct includes a sequence encoding a recombinant receptor including a STEAP1 binding domain. In particular embodiments, a genetic construct includes (i) a sequence encoding a pro-inflammatory cytokine, and (ii) a sequence encoding a recombinant receptor including a STEAP1 binding domain. In particular embodiments, the pro-inflammatory cytokine includes IL-18, IL1α, IL-1β, IL-33, IL-36α, IL-36β, or IL-36γ. In particular embodiments, the pro-inflammatory cytokine includes IL-18. In particular embodiments, the STEAP1 binding domain includes an scFv binding domain derived from a vandortuzumab vedotin (DSTP3086S; a humanized variant of the murine monoclonal antibody mAb 120.545). In particular embodiments, the recombinant receptor includes an extracellular component including the STEAP1 binding domain, an intracellular component, and a transmembrane domain linking the extracellular component to the intracellular component. In particular embodiments, the recombinant receptor is a CAR. In particular embodiments, the intracellular component includes a CD3z activation domain and a 4-1BB costimulatory domain. In particular embodiments, the extracellular domain includes a long spacer including the IgG4 hinge-CH2-CH3 with a 4/2-NQ mutation in the CH2 domain. In particular embodiments, the transmembrane domain includes a CD28 transmembrane domain. In particular embodiments, the genetic construct further includes a sequence encoding a self-cleaving polypeptide. In particular embodiments, the self-cleaving polypeptide includes a 2A peptide from Thosea asigna virus (T2A). In particular embodiments, the self-cleaving polypeptide includes a 2A peptide from porcine teschovirus-1 (P2A).

Aspects of the current disclosure are now described in more supporting detail as follows: (I) Immune Cells; (II) Cell Sample Collection and Cell Enrichment; (III) Genetically Modifying Cell Populations; (III-A) Genetic Engineering Techniques; (III-B) Pro-inflammatory Cytokines; (III-C) Recombinant Receptor Subcomponents; (III-C-i) Binding Domains; (III-C-ii) Spacers; (III-C-iii) Transmembrane Domains; (III-C-iv) Intracellular Effector Domains; (III-C-v) Linkers; (III-C-vi) Multimerization Domains; (III-D) Control Features Including Tag Cassettes, Transduction Markers, Selection Cassettes, and/or Suicide Switches; (IV) Characterization of Genetically Engineered Cells; (V) Cell Activating Culture Conditions; (VI) Ex Vivo Manufactured Cell Formulations; (VII) Targeted Viral Vectors & Nanoparticles; (VIII) Methods of Use; (IX) Reference Levels Derived from Control Populations; (X) Kits; (XI) Exemplary Embodiments; (XII) Experimental Examples; and (XIII) Closing Paragraphs. These headings are provided for organizational purposes only and do not limit the scope or interpretation of the disclosure.

(I) Immune Cells. The present disclosure describes cells genetically modified to express a recombinant receptor (e.g., CAR) and/or a pro-inflammatory cytokine. Genetically modified cells can include T-cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC). In particular embodiments, genetically modified cells include T-cells.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the αβ T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) as used herein refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+T lymphocytes are characterized by the expression of phenotypic markers of naive T-cells including CD62L, CCR7, CD28, CD127, and CD45RA.

Natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

NK cells include NK-T cells. NK-T cells are a specialized population of T cells that express a semi invariant T cell receptor (TCR ab) and surface antigens typically associated with natural killer cells. NK-T cells contribute to antibacterial and antiviral immune responses and promote tumor-related immunosurveillance or immunosuppression. Like natural killer cells, NK-T cells can also induce perforin-, Fas-, and TNF-related cytotoxicity. Activated NK-T cells are capable of producing IFN-γ and IL-4. In particular embodiments, NK-T cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Rα; and/or CD163.

Immature dendritic cells (i.e., pre-activation) engulf antigens and other non-self-components in the periphery and subsequently, in activated form, migrate to T-cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD24^{lo} Lin⁻ CD117⁺ hematopoietic progenitor cells. HPC can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. Preferably, the HSPC are CD34⁺ cells.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Cells to be genetically modified according to the teachings of the current disclosure can be patient-derived cells (autologous) or allogeneic when appropriate, and can also be in vivo or ex vivo. In particular embodiments, the immune cell is a CD4+ T cell or CD8+ T cell.

(II) Cell Sample Collection and Cell Enrichment. Methods of sample collection and enrichment are known by those skilled in the art. In some embodiments, cells are derived from cell lines. The cells in some embodiments are obtained from a xenogeneic source, for example, from mouse, rat, non-human primate, or pig. In particular embodiments, cells are derived from humans, for example a patient to be treated.

In some embodiments, T cells are derived or isolated from samples such as whole blood, peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T cells, monocytes, granulocytes, B cells, other nucleated white blood cells, HSC, HPC, HSPC, red blood cells, and/or platelets, and in some aspects contains cells other than red blood cells and platelets and further processing is necessary. In particular embodiments, T cells are derived from PBMCs.

In some embodiments, blood cells collected from a subject are washed, e.g., to remove the plasma fraction and to place the cells in an appropriate buffer or media for subsequent processing steps. In particular embodiments, the cells are washed with phosphate buffered saline (PBS). In some embodiments, the wash solution lacks calcium and/or magnesium and/or many or all divalent cations. Washing can be accomplished using a semi-automated “flow-through” centrifuge (for example, the Cobe 2991 cell processor, Baxter) according to the manufacturer's instructions. Tangential flow filtration (TFF) can also be performed. In particular embodiments, cells can be re-suspended in a variety of biocompatible buffers after washing, such as, Ca++/Mg++ free PBS.

The isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or engineering of the different populations is carried out from the same starting composition or material, such as from the same sample.

In particular embodiments, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, a selected T cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use. In particular embodiments, CD4+ and/or CD8+ T cells are enriched from PBMCs.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some examples, multiple rounds of separation steps are carried out, where the positively or negatively selected fraction from one step is subjected to another separation step, such as a subsequent positive or negative selection.

In some embodiments, an antibody or binding domain for a cellular marker is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, NJ); see also U.S. Pat. Nos. 4,452,773; 4,795,698; 5,200,084; and EP 452342.

In some embodiments, affinity-based selection is via magnetic-activated cell sorting (MACS) (Miltenyi Biotec, Auburn, CA). MACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1 (5): 355-376). In both cases, cells can be labeled with multiple markers, allowing for the isolation of well-defined cell subsets at high purity.

Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques.

CD3+, CD28+ T cells can be positively selected for and expanded using anti-CD3/anti-CD28 conjugated magnetic beads (e.g., DYNABEADS® (Life Technologies AS, Norway) M-450 CD3/CD28 T Cell Expander).

In particular embodiments, a CD8+ or CD4+ selection step is used to separate CD4+ helper and CD8+ cytotoxic T cells. Such CD8+ and CD4+ populations can be further sorted into sub-populations by positive or negative selection for markers expressed or expressed to a relatively higher degree on one or more naive, memory, and/or effector T cell subpopulations.

In some embodiments, enrichment for central memory T (TCM) cells is carried out. In particular embodiments, memory T cells are present in both CD62L subsets of CD8+ peripheral blood lymphocytes. PBMC can be enriched for or depleted of CD62L, CD8 and/or CD62L+CD8+ fractions, such as by using anti-CD8 and anti-CD62L antibodies.

In some embodiments, the enrichment for central memory T (TCM) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CCR7, CD45RO, and/or CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained, optionally following one or more further positive or negative selection steps.

Other cell types can be enriched based on known marker profiles and techniques. For example, CD34+ HSC, HSP, and HSPC can be enriched using anti-CD34 antibodies directly or indirectly conjugated to magnetic particles in connection with a magnetic cell separator, for example, the CliniMACS® Cell Separation System (Miltenyi Biotec, Bergisch Gladbach, Germany).

(III) Genetically Modifying Cell Populations. Cell populations can be genetically modified to express pro-inflammatory cytokines and/or recombinant receptors described herein.

(III-A) Genetic Engineering Techniques. Desired genes or genetic constructs disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence). The gene can encode a pro-inflammatory cytokine (e.g., IL-18) and/or the gene can encode a recombinant receptor including a STEAP1-binding domain. This definition includes various sequence polymorphisms, mutations, and/or sequence variants wherein such alterations do not substantially affect the function of the encoded genetic construct. The term “gene” may include not only coding sequences but also regulatory regions such as promoters, enhancers, and termination regions. Gene sequences encoding the molecule can be DNA or RNA that directs the expression of the open reading frame(s) within the genetic construct. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Portions of complete gene sequences are referenced throughout the disclosure as is understood by one of ordinary skill in the art.

Gene sequences encoding genetic constructs are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In embodiments, the gene sequence encoding the sequences can be codon optimized for expression in mammalian cells.

“Encoding” refers to the property of specific sequences of nucleotides in a gene, such as a cDNA, or an mRNA, to serve as templates for the synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Polynucleotide gene sequences encoding more than one portion of an expressed genetic constructs can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et al, Mol. Ther. 17 (8): 1453-1464 (2009). Other examples of lentivirus vectors that may be used in the clinic, include: the LENTIVECTOR® gene delivery technology from Oxford BioMedica, the LENTIMAX™ vector system from Lentigen and the like. Nonclinical types of lentiviral vectors are also available and would be known to one skilled in the art. In particular embodiments, cells are genetically engineered to express genetic constructs using a lentivirus or lentiviral vector.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

Retroviral vectors (see Miller, et al., 1993, Meth. Enzymol. 217:581-599) can be used. In such embodiments, the gene to be expressed is cloned into the retroviral vector for its delivery into cells. In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses, adeno-associated viruses (AAV) and alphaviruses can also be used. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19:673-686. Other methods of gene delivery include use of mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. A zinc finger is a domain of 30 amino acids within the zinc finger binding domain whose structure is stabilized through coordination of a zinc ion. Examples of zinc fingers include C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. A well-known example of a ZFN is a fusion of the FokI nuclease with a zinc finger DNA binding domain. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9 (8): 805-7; Ramirez et al., Nucl Acids Res, 2012, 40 (12): 5560-8; Kim et al., Genome Res, 2012, 22 (7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14 (I): 49-55; Beurdeley et al., Nat Commun, 2013, 4:1762; Scharenberg et al., Curr Gene Ther, 2013, 13 (4): 291-303; Gaj et al., Nat Methods, 2012, 9 (8): 805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments can use transposon-based systems as gene editing agents to mediate the integration of a genetic construct into cells. Generally, such methods will involve introducing into cells (i) a first vector encoding a transposase (or a transposase polypeptide) and (ii) a second vector encoding a desired genetic element that is flanked by transposon repeats. Transposons or transposable elements include a (short) nucleic acid sequence with terminal repeat sequences upstream and downstream thereof and encode enzymes that facilitate the excision and insertion of the nucleic acid into target DNA sequences.

Several transposon/transposase systems have been adapted for genetic insertions of heterologous DNA sequences. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON. Transposases and transposon systems are further described in U.S. Pat. Nos. 6,489,458; 7,148,203; 8,227,432; and 9,228,180.

(III-B) Pro-inflammatory Cytokines. A pro-inflammatory cytokine is a type of signaling molecule that is secreted by an immune cell or other cell to promote inflammation. Particular embodiments describe genetic constructs expressing a pro-inflammatory cytokine.

Cytokines are small proteins (generally 5-25 kDa) that are important in cell signaling. Cytokines are released by cells and affect the behavior of other cells, and sometimes the releasing cell itself, such as a T-cell. Cytokines can include, for example, chemokines, interferons, interleukins, lymphokines, and/or tumor necrosis factor. Cytokines can be produced by a broad range of cells, which can include, for example, immune cells like macrophages, B lymphocytes, T lymphocytes and/or mast cells, as well as, endothelial cells, fibroblasts, and/or various stromal cells.

Cytokines can act through receptors, and are important in the immune system as the cytokines can modulate the balance between humoral and cell-based immune responses, and can regulate the maturation, growth, and responsiveness of particular cell populations. Some cytokines enhance or inhibit the action of other cytokines in complex ways.

In particular embodiments, the pro-inflammatory cytokine includes interleukin (IL)-18, IL1α, IL-1β, IL-33, IL-36α, IL-36β, or IL-36γ.

The Interleukin-1 (IL-1) family consists of 11 structurally related family members, that are among the most potent immune system signaling molecules, acting through a group of closely related receptors. Seven ligands have agonist activity (IL-1α, IL-1β, IL-18, IL-33, IL-36α, IL-36β, IL-36γ) and four members with antagonistic activities [IL-1 receptor antagonist (IL-1Ra), IL-36Ra, IL-37, IL-38]. All IL-1 receptors have a similar mode of activation: upon binding of ligand to the primary receptor subunit (i.e. IL-1R1 for IL-1α and β, IL-18R for IL-18 and ST2 for IL-33), a second receptor subunit is recruited (i.e. IL-1RAP for IL-1α and β, IL-18RAP for IL-18 and IL-1RAP for IL-33) and signaling is initiated via juxtaposition of the receptor subunits' cytoplasmic Toll/IL-1 receptor (TIR) domains. The dimerized TIR domains provide a docking platform for the MYD88 adaptor protein, which via recruitment of other intermediates leads to activation of the pro-inflammatory nuclear factor-KB (NF-κB) and mitogen-activated protein kinase (MAPK) pathways. The IL-1 family members are primarily produced by innate immune cells and act on a variety of cell types during the immune response (for review see Sims and Smith, 2010).

Interleukin (IL)-18 is a pro-inflammatory cytokine involved in host defense against infections and regulates the innate and acquired immune response. IL-18 binds with high affinity and signals through the IL-18 receptor (IL-18R), a heteromeric complex of alpha and beta chains encoded by the genes IL18R1 and IL18RAP, respectively (Torigoe K et al (1997) J Biol Chem; 272 (41): 25737-42). IL-18 has been shown to mediate a variety of autoimmune and inflammatory diseases. It has been demonstrated that IL-18 expression is up-regulated in several autoimmune diseases, such as Crohn's disease, psoriasis, rheumatoid arthritis, multiple sclerosis and cardiovascular diseases (Braddock et al. (2004) Expert Opin Biol Ther; 4 (6): 847 860). IL-18 is also up-regulated in certain inflammatory diseases such as chronic obstructive pulmonary disease (COPD) (Imaoka et al. (2008) Eur Respir; J31: 287-297), idiopathic pulmonary fibrosis (IPF) (Kitasato et al. (2004) Am J Resp Cell Mol Biol; 31:619-625), macrophage activation syndrome (MAS) (Dinarello and Kaplanski (2005) Expert Rev Clin Immunol; 1 (4): 619-632), adult onset Still's disease (AOSD) (Arlet J B et al. (2006) Ann Rheum Dis 65 (12): 1596-601) and systemic juvenile idiopathic arthritis (SJIA) (Akashi et al. (1994) Br J Haematol; 87 (2): 243-50).

In particular embodiments, human IL-18 (hIL-18) includes the sequence:

(SEQ ID NO: 4)
YFGKLESKLSVIRNLNDQVLFIDQGNRPLFEDMTDSDCRDNAPRTIFIIS
MYKDSQPRGMAVTISVKCEKISTLSCENKIISFKEMNPPDNIKDTKSDII
FFQRSVPGHDNKMQFESSSYEGYFLACEKERDLFKLILKKEDELGDRSIM
FTVQNED.

In particular embodiments, hIL-18 is encoded by the sequence:

(SEQ ID NO: 5)
TACTTTGGCAAGCTTGAATCTAAATTATCAGTCATAAGAAATTTGAATGA
CCAAGTTCTCTTCATTGACCAAGGAAATCGGCCTCTATTTGAAGATATGA
CTGATTCTGACTGTAGAGATAATGCACCCCGGACCATATTTATTATAAGT
ATGTATAAAGATAGCCAGCCTAGAGGTATGGCTGTAACTATCTCTGTGAA
GTGTGAGAAAATTTCAACTCTCTCCTGTGAGAACAAAATTATTTCCTTTA
AGGAAATGAATCCTCCTGATAACATCAAGGATACAAAAAGTGACATCATA
TTCTTTCAGAGAAGTGTCCCAGGACATGATAATAAGATGCAATTTGAATC
TTCATCATACGAAGGATACTTTCTAGCTTGTGAAAAAGAGAGAGACCTTT
TTAAACTCATTTTGAAAAAAGAGGATGAATTGGGGGATAGATCTATAATG
TTCACTGTTCAAAACGAAGAC

In particular embodiments, IL-18 includes the sequence:

(SEQ ID NO: 6)
MAAEPVEDNCINFVAMKFIDNTLYFIAEDDENLESDYFGKLESKLSVIRN
LNDQVLFIDQGNRPLFEDMTDSDCRDNAPRTIFIISMYKDSQPRGMAVTI
SVKCEKISTLSCENKIISFKEMNPPDNIKDTKSDIIFFQRSVPGHDNKMQ
FESSSYEGYFLACEKERDLFKLILKKEDELGDRSIMFTVQNED.

IL1α and IL-1β are members that laid the foundation for the IL1 cytokine family. Both are pleiotropic and function through a widely expressed receptor complex composed of IL-1 receptor type I (IL-1RI) and IL-1 receptor accessory protein (IL-1RAcP) To do. This overactivation of IL-1 is associated with a number of diseases including rheumatoid arthritis (RA), chronic obstructive pulmonary disease (COPD), asthma, inflammatory bowel disease, multiple sclerosis, atherosclerosis and Alzheimer's disease. It is associated with human pathology. Many different lines of immune cells, including innate immune cells such as dendritic cells, macrophages and neutrophils, as well as cells involved in the adaptive immune response, including naïve cells, Th17 and CD8+ T cells, and B cells are It is activated by IL-1 (reviewed in Sims and Smith, 2010).

In particular embodiments, IL1α includes the sequence:

(SEQ ID NO: 7)
MAKVPDMFEDLKNCYSENEEDSSSIDHLSLNQKSFYHVSYGPLHEGCMDQ
SVSLSISETSKTSKLTFKESMVVVATNGKVLKKRRLSLSQSITDDDLEAI
ANDSEEEIIKPRSSPFSFLSNVKYNFMRIIKYEFILNDALNQSIIRANDQ
YLTAAALHNLDEAVKFDMGAYKSSKDDAKITVILRISKTQLYVTAQDEDQ
PVLLKEMPEIPKTITGSETNLLFFWETHGTKNYFTSVAHPNLFIATKQDY
WVCLAGGPPSITDFQILENQA.

In particular embodiments, IL-1β includes the sequence:

(SEQ ID NO: 8)
MAEVPELASEMMAYYSGNEDDLFFEADGPKQMKCSFQDLDLCPLDGGIQL
RISDHHYSKGFRQAASVVVAMDKLRKMLVPCPQTFQENDLSTFFPFIFEE
EPIFFDTWDNEAYVHDAPVRSLNCTLRDSQQKSLVMSGPYELKALHLQGQ
DMEQQVVFSMSFVQGEESNDKIPVALGLKEKNLYLSCVLKDDKPTLQLES
VDPKNYPKKKMEKRFVFNKIEINNKLEFESAQFPNWYISTSQAENMPVFL
GGTKGGQDITDFTMQFVSS.

IL-33 is a pro-inflammatory cytokine released by damaged epithelial tissue in response to insults such as allergens, viruses, or smoke. It is a member of the interleukin-1 (IL-1) family that potently induces the production of T helper-2 (Th2)-associated cytokines (e.g., IL-4). IL-33 is expressed by a wide variety of cell types including fibroblasts, mast cells, dendritic cells, macrophages, osteoblasts, endothelial cells and epithelial cells. Interleukin-33 (IL-33) is a ligand for the Toll-like/interleukin-1 receptor superfamily member ST2 (sometimes referred to as “suppression of tumorigenicity 2”). and the ST2 is the accessory protein IL-1RAcP (“interleukin-1 receptor accessory protein”). Activation of ST2/IL-1RAcP by IL-33 triggers a signaling cascade through downstream molecules such as MyD88 (myeloid differentiation factor 88) and TRAF6 (TNF receptor-associated factor 6), in particular It leads to activation of NFκB (nuclear factor-κB). IL-33 signaling has been implicated as a factor in various diseases and disorders, including the inflammatory lung diseases.

In particular embodiments, IL-33 includes the sequence:

(SEQ ID NO: 9)
MKPKMKYSTNKISTAKWKNTASKALCFKLGKSQQKAKEVCPMYFMKLRSG
LMIKKEACYFRRETTKRPSLKTGRKHKRHLVLAACQQQSTVECFAFGISG
VQKYTRALHDSSITGISPITEYLASLSTYNDQSITFALEDESYEIYVEDL
KKDEKKDKVLLSYYESQHPSNESGDGVDGKMLMVTLSPTKDFWLHANNKE
HSVELHKCEKPLPDQAFFVLHNMHSNCVSFECKTDPGVFIGVKDNHLALI
KVDSSENLCTENILFKLSET.

IL-36α, IL-36β, and IL-36γ are highly expressed in several tissues, including internal epithelial tissue and skin that have been exposed to pathogens. Expression of IL-36Ra and IL-36α was significantly upregulated in IL-1β/TNF-α stimulated human keratinocytes and IL-36Rα and IL-36γ mRNA is overexpressed in psoriatic skin lesions. Elevated IL-36a mRNA and protein expression has also been observed in chronic kidney disease (Ichii et al., Lab Invest., 90 (3): 459-475 (2010)). Both mouse bone marrow-derived dendritic cells (BMDCs) and CD4+T lymphocytes constitutively express IL-36R, an inflammatory cytokine that induces more potent stimulatory effects than other IL-1 cytokines (e.g., IL-12, IL-1β, IL-6, TNF-α, and IL-23) and reacts directly with IL-36α, IL-36β, and IL-36γ (Vigne et al., Blood, 118). (22): 5813-5823 (2011)).

In particular embodiments, IL-36α includes the sequence:

(SEQ ID NO: 10)
MEKALKIDTPQQGSIQDINHRVWVLQDQTLIAVPRKDRMSPVTIALISCR
HVETLEKDRGNPIYLGLNGLNLCLMCAKVGDQPTLQLKEKDIMDLYNQPE
PVKSFLFYHSQSGRNSTFESVAFPGWFIAVSSEGGCPLILTQELGKANTT
DFGLTMLF.

In particular embodiments, IL-36β isoform 1 includes the sequence:

(SEQ ID NO: 11)
MNPQREAAPKSYAIRDSRQMVWVLSGNSLIAAPLSRSIKPVTLHLIACRD
TEFSDKEKGNMVYLGIKGKDLCLFCAEIQGKPTLQLKLQGSQDNIGKDTC
WKLVGIHTCINLDVRESCFMGTLDQWGIGVGRKKWKSSFQHHHLRKKDKD
FSSMRTNIGMPGRM.

In particular embodiments, IL-36β isoform 2 includes the sequence:

(SEQ ID NO: 12)
MNPQREAAPKSYAIRDSRQMVWVLSGNSLIAAPLSRSIKPVTLHLIACRD
TEFSDKEKGNMVYLGIKGKDLCLFCAEIQGKPTLQLKEKNIMDLYVEKKA
QKPFLFFHNKEGSTSVFQSVSYPGWFIATSTTSGQPIFLTKERGITNNTN
FYLDSVE.

In particular embodiments, IL-36γ includes the sequence:

(SEQ ID NO: 13)
MRGTPGDADGGGRAVYQSMCKPITGTINDLNQQVWTLQGQNLVAVPRSDS
VTPVTVAVITCKYPEALEQGRGDPIYLGIQNPEMCLYCEKVGEQPTLQLK
EQKIMDLYGQPEPVKPFLFYRAKTGRTSTLESVAFPDWFIASSKRDQPII
LTSELGKSYNTAFELNIND.

(III-C) Recombinant Receptor Subcomponents. In particular embodiments, a recombinant receptor is or includes a binding domain that binds a target antigen, wherein the recombinant receptor is expressed by a cell following the artificial introduction of nucleic acid encoding the recombinant receptor into the cell. The recombinant receptor can be, e.g., a CAR, an engineered T cell receptor (eTCR), or a hybrid thereof.

As described previously, CAR molecules include several distinct subcomponents that allow genetically modified cells to recognize and kill unwanted cells, such as cancer cells. The subcomponents include at least an extracellular component and an intracellular component. The extracellular component includes a binding domain that specifically binds a marker that is preferentially present on the surface of unwanted cells. When the binding domain binds such markers, the intracellular component activates the cell to destroy the bound cell. CAR additionally include a transmembrane domain that links the extracellular component to the intracellular component, and other subcomponents that can increase the CAR's function. For example, the inclusion of a spacer and/or one or more linker sequences can allow the CAR to have additional conformational flexibility, often increasing the binding domain's ability to bind the targeted antigen. Each of these subcomponents is described in more detail in the following section (III-C) subheadings.

Many considerations associated with CAR apply to eTCR as well. eTCR disclosed herein include a binding domain that binds a target antigen (e.g., an scFv) linked to the Ca and/or CB chains of a TCR. A TCR is a heterodimeric fusion protein that typically includes an α and β chain. Each chain includes a variable region (Vα and Vβ) and a constant region (Cα and Cβ). In particular embodiments, an eTCR does not include the native TCR variable region but does include the native TCR constant region. In particular embodiments, the eTCR includes an scFv as the variable region of either the α or β chain. In particular embodiments, the eTCR includes an scFv as the variable region of both the α and β chain. In particular embodiments, eTCR include a Cα and/or Cβ chain sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR Cα or Cβ.

For particular examples of TCR that can be used within the context of the current disclosure, see, for example, WO2018/129270; WO2017/112944; WO2011/039507; U.S. Pat. No. 8,008,438; US2016/0083449; US2015/0246959; Stromnes, et al. (2015) Cancer cell 28 (5): 638-652; Kobayashi, et al. (2013) Nature Medicine 19:1542-1546); Varela-Rohena, et al. (2008) Nature Medicine. 14 (12): 1390-1395); and Robbins et al. (2008) The Journal of Immunology 180 (9): 6116-6131.

(III-C-i) Binding Domains. In certain examples, the current disclosure provides binding domains for use in CAR based on antibodies that bind STEAP1. Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR). The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al. (1997) J Mol Biol 273:927-948 (Chothia numbering scheme); Maccallum et al. (1996) J Mol Biol 262:732-745 (Contact numbering scheme); Martin et al. (1989) Proc. Natl. Acad. Sci., 86:9268-9272 (AbM numbering scheme); North et al. (2011) J. Mol. Biol. 406 (2): 228-56 (North numbering scheme); Lefranc M P et al. (2003) Dev Comp Immunol 27 (1): 55-77 (IMGT numbering scheme); and Honegger and Pluckthun (2001) J Mol Biol 309 (3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering. North numbering uses longer sequences in the structural analysis of the conformations of CDR loops. CDR residues can be identified using software programs such as ABodyBuilder.

In particular embodiments, a STEAP1 binding domain includes a CDR set provided herein. A CDR set refers to 3 heavy chain CDRs and 3 light chain CDRs that together result in binding to STEAP1.

TABLE 1
STEAP1 Binding Domain CDR Sequences.

| CDR | | | SEQ |
|---|---|---|---|
| Definition | CDR | Sequence | ID NO: |
| Kabat | CDRH1 | SDYAWN | 14 |
| | CDHR2 | YISNSGSTSYNPSLKS | 15 |
| | CDRH3 | ERNYDYDDYYYAMDY | 16 |
| | CDRL1 | KSSQSLLYRSNQKNYLA | 17 |
| | CDRL2 | WASTRES | 18 |
| | CDRL3 | QQYYNYPRT | 19 |
| Chothia | CDRH1 | GYSITSDY | 20 |
| | CDHR2 | SNSGS | 21 |
| | CDRH3 | ERNYDYDDYYYAMDY | 16 |
| | CDRL1 | KSSQSLLYRSNQKNYLA | 17 |
| | CDRL2 | WASTRES | 18 |
| | CDRL3 | QQYYNYPRT | 19 |
| IMGT | CDRH1 | GYSITSDYA | 22 |
| | CDHR2 | ISNSGST | 23 |
| | CDRH3 | ARERNYDYDDYYYAMDY | 24 |
| | CDRL1 | QSLLYRSNQKNY | 25 |
| | CDRL2 | WAS | N/A |
| | CDRL3 | QQYYNYPRT | 19 |
| North | CDRH1 | AVSGYSITSDYAWN | 26 |
| | CDHR2 | YISNSGSTS | 27 |
| | CDRH3 | ARERNYDYDDYYYAMDY | 24 |
| | CDRL1 | KSSQSLLYRSNQKNYLA | 17 |
| | CDRL2 | YWASTRES | 28 |
| | CDRL3 | QQYYNYPRT | 19 |
| Contact | CDRH1 | TSDYAWN | 29 |
| | CDHR2 | WVGYISNSGSTS | 30 |
| | CDRH3 | ARERNYDYDDYYYAMD | 31 |
| | CDRL1 | KNYLAWY | 32 |
| | CDRL2 | LLIYWASTRE | 33 |
| | CDRL3 | QQYYNYPR | 34 |

In some instances, additional scFvs based on the binding domains described herein can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. scFv are commonly used as the binding domains of recombinant receptors (e.g., CAR). In particular embodiments, the recombinant receptor includes a binding domain that binds STEAP1. In particular embodiments, the binding domain that binds STEAP1 (or STEAP1 binding domain) is an scFv. In particular embodiments, the binding domain that binds STEAP1 is an scFV derived from vandortuzumab vedotin (DSTP3086S). In particular embodiments, the binding domain that binds STEAP1 is a humanized variant of the murine monoclonal antibody mAb 120.545.

In particular embodiments, the STEAP1 binding domain includes a variable heavy chain including the sequence:
EVOLVESGGGLVQPGGSLRLSCAVSGYSITSDYAWNWVRQAPGKGLEWVGYISNSGSTSYN PSLKSRFTISRDNSKNTLYLQMNSLRAEDTAVYYCARERNYDYDDYYYAMDYWGQGTLVTVS S (SEQ ID NO: 35) and a variable light chain including the sequence:
DIQMTQSPSSLSASVGDRVTITCKSSQSLLYRSNQKNYLAWYQQKPGKAPKLLIYWASTRESG VPSRFSGSGSGTDFTLTISSLQPEDFATYYCQQYYNYPRTFGQGTKVEIKR (SEQ ID NO: 36).

In particular embodiments, the STEAP1 binding domain includes a variable heavy chain encoded by the sequence as set forth in SEQ ID NO: 72 and a variable light chain encoded by the sequence as set forth in SEQ ID NO: 70.

In particular embodiments, the STEAP1 binding domain is an scFv including the sequence as set forth in SEQ ID NO: 73 or SEQ ID NO: 75. In particular embodiments, the STEAP1 binding domain is an scFV encoded by the sequence as set forth in SEQ ID NO: 74 or SEQ ID NO: 76.

In particular embodiments, the STEAP1 binding domain can be derived from commercially available antibodies such as 4F6-1F3, MAB55871-100, 20199-1-AP, AF5587, ABIN6746136, ab207914, 600-401-EV6, or ABIN525786.

Other binding fragments, such as Fv, Fab, Fab′, F(ab′)2, can also be used within the recombinant receptor disclosed herein. Additional examples of antibody-based binding domain formats for use in a recombinant receptor include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In particular embodiments, the binding domain includes a humanized antibody or an engineered fragment thereof. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable domain. As provided herein, humanized antibodies or antibody fragments include one or more CDRs from nonhuman immunoglobulin molecules and framework regions wherein the amino acid residues including the framework are derived completely or mostly from human germline. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28 (4/5): 489-498; Studnicka et al., 1994, Protein Engineering, 7 (6): 805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13 (5): 353-60 (2000), Morea et al., Methods, 20 (3): 267-79 (2000), Baca et al., J. Biol. Chem., 272 (16): 10678-84 (1997), Roguska et al., Protein Eng., 9 (10): 895-904 (1996), Couto et al., Cancer Res., 55 (23 Supp): 5973s-5977s (1995), Couto et al., Cancer Res., 55 (8): 1717-22 (1995), Sandhu J S, Gene, 150 (2): 409-10 (1994), and Pedersen et al., J. Mol. Biol., 235 (3): 959-73 (1994). Often, framework residues in the framework regions will be substituted with the corresponding residue from the CDR donor antibody to alter, for example improve, STEAP1 binding. These framework substitutions are identified by methods well-known in the art, e.g., by modeling of the interactions of the CDR and framework residues to identify framework residues important for STEAP1 binding and sequence comparison to identify unusual framework residues at particular positions. (See, e.g., U.S. Pat. No. 5,585,089; and Riechmann et al., 1988, Nature, 332:323).

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling components) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a VL region in a binding domain of the present disclosure is derived from or based on a VL of an antibody disclosed herein and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the antibody disclosed herein. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of an antibody disclosed herein and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the antibody disclosed herein. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from an antibody disclosed herein or fragment or derivative thereof that specifically binds to STEAP1.

(III-C-ii) Transmembrane Domains. As indicated, transmembrane domains within a recombinant receptor serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 CD154, Toll-like receptor 1 (TLR1), TLR2, TLR3, TLR4, TLR5, TLR6, TLR7, TLR8, and TLR9. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge. In particular embodiments, the recombinant receptor includes a CD28 transmembrane domain. It has been shown that a CD28 transmembrane domain reduces the antigen-threshold for second-generation 4-1BB CAR T cell activation.

In particular embodiments, a transmembrane domain has a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof.

A transmembrane domain can include one or more additional amino acids adjacent to the transmembrane region, e.g., one or more amino acid within the extracellular region of the recombinant receptor (e.g., up to 15 amino acids of the extracellular region) and/or one or more additional amino acids within the intracellular region of the recombinant receptor (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the recombinant receptor is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is encoded by the nucleic acid sequence encoding the CD28 transmembrane domain (SEQ ID NOs: 102, 103, 104, or 105). In particular embodiments, the transmembrane domain includes the amino acid sequence of the CD28 transmembrane domain (SEQ ID NOs: 99, 100, or 101).

(III-C-iv) Intracellular Effector Domains. The intracellular effector domains of a recombinant receptor are responsible for activation of the cell in which the recombinant receptor is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD30, CD3E, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, NKp46, TLR1, TLR2, TLR3, TLR4, TLR5, TLR6, TLR7, TLR8, or TLR9. In particular embodiments, the effector domain includes a CD3ζ signaling domain.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119 (3): 696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CDIId, ITGAE, CD103, ITGAL, CDIIa, ITGAM, CDIIb, ITGAX, CDIIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, and CD19a. In particular embodiments, the co-stimulatory domain includes a 4-1BB signaling domain.

In particular embodiments, the intracellular signaling components include CD3ζ (encoded by, for example, SEQ ID NOs: 91 or 92) and/or a variant of 4-1BB (encoded by, for example, SEQ ID NOs: 96, 97, or 98). In particular embodiments, the intracellular signaling components include a variant of CD3ζ (SEQ ID NOs: 89, 90, or 91) and/or a portion of 4-1BB (SEQ ID NOs: 93, 94, or 95).

In particular embodiments, the intracellular signaling component includes (i) all or a portion of the signaling domain of CD3ζ, (ii) all or a portion of the signaling domain of 4-1BB, or (iii) all or a portion of the signaling domain of CD3ζ and 4-1BB. In particular embodiments, the intracellular signaling component includes (i) all or a portion of the signaling domain of CD3ζ, (ii) all or a portion of the signaling domain of 4-1BB, (iii) all or a portion of the signaling domain of CD28, or (iv) all or a portion of the signaling domain of CD3ζ, 4-1BB, and CD28.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

(III-C-iv) Multimerization Domains. In particular embodiments, the recombinant receptor can optionally include a multimerization domain. Protein biological activities depend upon their tertiary and quaternary structure. The quaternary structure requires the physical and chemical interaction of different protein subunits or polypeptides. A “multimerization domain” is a domain that causes two or more proteins (monomers) to interact with each other through covalent and/or non-covalent association(s). Multimerization domains present in proteins can result in protein interactions that form dimers, trimers, tetramers, pentamers, hexamers, heptamers, etc., depending on the number of units/monomers incorporated into the multimer.

In particular embodiments, the multimerization domain is a dimerization domain that allows binding of two complementary monomers to form a dimer. In particular embodiments, complementary monomers include PRKAR1A and PRKAR1A (SEQ ID NOs: 159 and 160), PRKAR1B and PRKAR1B (SEQ ID NO: 161), PRKAR1R (SEQ ID NOs: 162 and 163) and PRKAR1E (SEQ ID NO: 164). In particular embodiments, a dimerization and docking domain (DDD) can be derived from the cAMP-dependent protein kinase (PKA) regulatory subunits and can be paired with an anchoring domain (AD). The AD can be derived from a specific region found in various A-kinase anchoring proteins (AKAPs) that mediates association with the R subunits of PKA. In particular embodiments, complementary monomers include DDD (SEQ ID NOs: 165 and 166) and AD (SEQ ID NOs: 167 and 168). One skilled in the art will realize that other DDDs and ADs are known and can be used such as: the 4-helix bundle type DDD (Newlon, et al. EMBO J. 2001; 20:1651-1662; Newlon, et al. Nature Struct Biol. 1999; 3:222-227) domains obtained from p53, DCoH (pterin 4 α carbinolamine dehydratase/dimerization cofactor of hepatocyte nuclear factor 1α (TCF1)) and HNF-1 (hepatocyte nuclear factor 1) (Rose, et al. Nature Struct Biol. 2000; 7:744-748). Other AD sequences of potential use may be found in US 2003/0232420A1.

In particular embodiments, complementary binding domains can dimerize. In particular embodiments, the binding domain is a transmembrane polypeptide derived from a FcεRI chain. In particular embodiments, a recombinant receptor can include a part of a FcεRI α chain and another recombinant receptor can include a part of an FcεRI β chain such that said FcεRI chains spontaneously dimerize together to form a dimeric recombinant receptor. In particular embodiments, recombinant receptor can include a part of a FcεRI α chain and a part of a FcεRI γ chain such that said FcεRI chains spontaneously trimerize together to form a trimeric recombinant receptor, and in another embodiment the multi-chain recombinant receptor can include a part of FcεRI α chain, a part of FcεRI β chain and a part of FcεRI γ chain such that said FcεRI chains spontaneously tetramerize together to form a tetrameric recombinant receptor.

In particular embodiments, complementary binding domains can be derived from binding events such as those between an enzyme and its substrate/inhibitor, for example, cutinase and phosphonates (Hodneland, et al. Proc Natl Acd Sci USA. 2002; 99:5048-5052), may also be utilized to generate the two associating components (the “docking” step), which are subsequently stabilized covalently (the “lock” step).

In particular embodiments, binding domains can be derived from binding events such as those between receptor dimer pair such as the interleukin-8 receptor (IL-8R), integrin heterodimers such as LFA-I and GPIIIb/IIIa, dimeric ligand polypeptides such as nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa et al., J Biol. Chem., 269:27833-27839, 1994; Radziejewski et al., Biochem, 32:1350, 1993) and variants of some of these domains with modified affinities, such as those described in WO2012001647A2.

In particular embodiments, dimerization domains can include protein sequence motifs such as coiled coils, acid patches, zinc fingers, calcium hands, a CH1-CL pair, an “interface” with an engineered “knob” and/or “protuberance” (U.S. Pat. No. 5,821,333), leucine zippers (U.S. Pat. No. 5,932,448), SH2 and SH3 (Vidal et al., Biochemistry, 43:7336-44, 2004), PTB (Zhou et al., Nature, 378:584-592, 1995), WW (Sudol Prog Biochys MOL Bio, 65:113-132, 1996), PDZ (Kim et al., Nature, 378:85-88, 1995; Komau et al., Science, 269:1737-1740, 1995) and WD40 (Hu et al., J Biol Chem., 273:33489-33494, 1998).

In particular embodiments, the sequence corresponding to a dimerization domain includes the leucine zipper domain of Jun (SEQ ID NO: 169), the dimerization domain of Fos (SEQ ID NO: 170), a consensus sequence for a WW motif (SEQ ID NO: 171), the dimerization domain of the SH2B adapter protein from GenBank Accession no. AAF73912.1 (Nishi et al., Mol Cell Biol, 25:2607-2621, 2005; SEQ ID NO: 172), the SH3 domain of IB1 from GenBank Accession no. AAD22543.1 (Kristensen et al., EMBO J., 25:785-797, 2006; SEQ ID NO: 173), the PTB domain of human DOK-7 from GenBank Accession no. NP_005535.1 (Wagner et al., Cold Spring Harb Perspect Biol. 5: a008987, 2013; SEQ ID NO: 174), the PDZ-like domain of SATB1 from UniProt Accession No. Q01826 (Galande et al., Mol Cell Biol. August; 21:5591-5604, 2001; SEQ ID NO: 175), the WD40 repeats of APAF from UniProt Accession No. 014727 (Jorgensen et al., 2009. PLOS One. 4 (12): e8463; SEQ ID NO: 176), the PAS motif of the dioxin receptor from UniProt Accession No. 16L9E7 (Pongratz et al., Mol Cell Biol, 18:4079-4088, 1998; SEQ ID NO: 177) and the EF hand motif of parvalbumin from UniProt Accession No. P20472 (Jamalian et al., Int J Proteomics, 2014:153712, 2014; SEQ ID NO: 178).

In particular embodiments, complementary binding domains can be induced using a third molecule or chemical inducer. This method of dimerization requires that one recombinant receptor or component thereof include a chemical inducer of dimerization binding domain 1 (CBD1) and the second recombinant receptor or component thereof include the second chemical inducer of dimerization binding domain (CBD2), wherein CBD1 and CBD2 are capable of simultaneously binding to a chemical inducer of dimerization (CID). CBD1 may include a rapamycin binding domain of FK-binding protein 12 (FKBP12) (SEQ ID NO: 179) and CBD2 may include a FKBP12-Rapamycin Binding (FRB) domain of mTOR (SEQ ID NO: 180). In this case, the CID can include rapamycin or a derivative thereof which is capable of causing CBD1 and CBD2 to heterodimerize. If CBD1 and CBD2 are a FK506 (Tacrolimus) binding domain of FKBP12 and a cyclosporin binding domain of cylcophilin A, the CID can include a FK506/cyclosporin fusion protein. If CBD1 and CBD2 are FKBP12 binding domains including a F36V mutation, the CID can be AP1903. If CBD1 and CBD2 are an oestrogen-binding domain (EBD) and a streptavidin binding domain, the CID can be an estrone/biotin fusion protein. If CBD1 and CBD2 are a glucocorticoid-binding domain (GBD) and a dihydrofolate reductase (DHFR) binding domain, the CID can be a dexamethasone/methotrexate fusion molecule. If CBD1 and CBD2 are an O⁶-alkylguanine-DNA alkyltransferase (AGT) binding domain and a DHFR binding domain, the CID can be an O⁶-benzylguanine derivative/methotrexate fusion molecule. If CBD1 and CBD2 are a retinoic acid receptor domain and an ecodysone receptor domain, the CID can include RSL1. Use of the CID binding domains can also be used to alter the affinity to the CID. For instance, altering amino acids at positions 2095, 2098, and 2101 of FRB can alter binding to Rapamycin (Bayle et al, Chemistry & Biology 13, 99-107, 2006).

C4b multimerization domains can also be used. Particular C4b multimerization domains that can be used are provided as SEQ ID NOs: 181-213. In particular embodiments, the C4b multimerization domain will be a multimerization domain which includes (i) glycine at position 12, (ii) alanine at position 28, (iii) leucines at positions 29, 34, 36, and/or 41; (iv) tyrosine at position 32; (v) lysine at position 33; and/or (vi) cysteine at positions 6 and 18. In particular embodiments, the C4b multimerization domain will be a multimerization domain which includes (i) glycine at position 12, (ii) alanine at position 28, (iii) leucines at positions 29, 34, 36, and 41; (iv) tyrosine at position 32; (v) lysine at position 33; and (vi) cysteine at positions 6 and 18.

C4b multimerization domains can include any of SEQ ID NOs: 181-213 with an N-terminal deletion of at least 1 consecutive amino acid residue (e.g., at least 2, 3, 4, 5, 6, 7, 8, 9, 10 consecutive amino acid residues) in length. Additional embodiments can include a C-terminal deletion of at least 1 consecutive amino acid residue (e.g., at least 2, 3, 4, 5, 6, 7, 8, 9, 10 consecutive amino acid residues) in length.

Particular C4b multimerization domain embodiments will retain or will be modified to include at least 1 of the following residues: A6; E11; A13; D21; C22; P25; A27; E28; L29; R30; T31; L32; L33; E34; I35; K37; L38; L40; E41; I42; Q43; K44; L45; E48; L49; or Q50. Further embodiments will retain or will be modified to include A6; E11; A13; D21; C22; P25; A27; E28; L29; R30; T31; L32; L33; E34; I35; K37; L38; L40; E41; I42; Q43; K44; L45; E48; L49; and Q50. Particular C4b multimerization domain embodiments will include the amino acid sequence “AELR”.

In particular embodiments, dextrameric and ferritin-based multimerization can be used. An exemplary ferritin fusion sequence is described in PMID 26279189.

In particular embodiments, additional methods of causing dimerization can be utilized. Additional modifications to generate a dimerization domain in a recombinant receptor could include: generating a second interchain disulfide bond in the C-terminus domain by introducing a second cysteine residue into both recombinant receptors; swapping interacting residues in each of the recombinant receptor constructs in the C-terminus domains (“knob-in-hole”); and fusing the variable domains of the recombinant receptor directly to CD3ζ (CD3ζ fusion) (Schmitt et al., Hum. Gene Ther. 2009. 20:1240-1248).

(III-D) Spacers and Linkers. Spacers are used to create appropriate distances and/or flexibility between components (e.g., CAR sub-components). In particular embodiments, the recombinant receptor includes a spacer. Linkers, alternatively, can include any portion of a molecule that serves to connect two subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of a recombinant receptor.

In particular embodiments, the length of a spacer is customized for binding STEAP1-expressing cells and mediating destruction. In particular embodiments, a spacer length can be selected based upon the location of a cellular marker epitope, affinity of a binding domain for the epitope, and/or the ability of the STEAP1-binding agent to mediate cell destruction following STEAP1 binding.

Spacers typically include those having 10 to 250 amino acids, 10 to 200 amino acids, 10 to 150 amino acids, 10 to 100 amino acids, 10 to 50 amino acids, or 10 to 25 amino acids.

In particular embodiments, a spacer is 5 amino acids, 8 amino acids, 10 amino acids, 12 amino acids, 14 amino acids, 20 amino acids, 21 amino acids, 26 amino acids, 27 amino acids, 45 amino acids, 50 amino acids, or 75 amino acids. These lengths qualify as short spacers.

In particular embodiments, a spacer is 76 amino acids, 90 amino acids, 100 amino acids, 110 amino acids, 120 amino acids, 125 amino acids, 128 amino acids, 131 amino acids, 135 amino acids, 140 amino acids, 150 amino acids, 160 amino acids, 170 amino acids, or 179 amino acids. These lengths qualify as intermediate spacers.

In particular embodiments, a spacer is 180 amino acids, 190 amino acids, 200 amino acids, 210 amino acids, 220 amino acids, 228 amino acids, 230 amino acids, 240 amino acids, 250 amino acids, 260 amino acids, or 270 amino acids. These lengths qualify as long spacers.

Exemplary spacers include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular embodiments, the spacer is a short spacer, an intermediate (or medium) spacer, or a long spacer. In particular embodiments, the short spacer includes an IgG4 hinge region. In particular embodiments, the intermediate spacer includes an IgG4 hinge region and an IgG4 CH3 domain. In particular embodiments, the long spacer includes an IgG4 hinge region, an IgG4 CH2 domain, and an IgG4 CH3 domain.

In particular embodiments the IgG4 hinge region is encoded by any of SEQ ID NOs: 78, 79, or 80. In particular embodiments, the IgG4 hinge region includes the sequence as set forth in SEQ ID NO: 77. In particular embodiments, the IgG4-CH2 domain is encoded by the sequence as set forth in SEQ ID NO: 82. In particular embodiments, the IgG4-CH2 domain includes the sequence as set forth in SEQ ID NO: 81. In particular embodiments, the IgG4-CH3 domain is encoded by the sequence as set forth in SEQ ID NO: 85. In particular embodiments, the IgG4-CH3 domain includes the sequence as set forth in SEQ ID NO: 84.

In particular embodiments the short spacer is encoded by any of SEQ ID NOs: 78, 79, or 80. In particular embodiments, the short spacer includes the sequence as set forth in SEQ ID NO: 77. In particular embodiments the intermediate spacer is encoded by SEQ ID NO: 86 In particular embodiments the long spacer is encoded by SEQ ID NO: 88. In particular embodiments the long spacer includes the sequence as set forth in SEQ ID NO: 87. In particular embodiments, the long spacer includes a 4/2-N/Q mutation in the CH2 domain. Mutations can be used to prevent Fc-gamma receptor binding and activation-induced cell death.

Other examples of hinge regions that can be used as a spacer described herein include the hinge region present in the extracellular regions of type 1 membrane proteins, such as CD8a, CD4, CD28 and CD7, which may be wild-type or variants thereof.

In particular embodiments, a spacer includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

Linkers can be flexible, rigid, or semi-rigid, depending on the desired function of the linker. Linkers can include junction amino acids. For example, in particular embodiments, linkers provide flexibility and room for conformational movement between different components of a recombinant receptor. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 37), (Gly₃Ser)ₙ (Gly₄Ser)ₙ (SEQ ID NO: 38), (Gly₃Ser)ₙ (Gly₂Ser)ₙ (SEQ ID NO: 39), or (Gly₃Ser)ₙ (Gly₄Ser)₁ (SEQ ID NO: 40). In particular embodiments, the linker is (Gly₄Ser)₄ (SEQ ID NO: 41), (Gly₄Ser)₃ (SEQ ID NO: 42), (Gly₄Ser)₂ (SEQ ID NO: 43), (Gly₄Ser)₁ (SEQ ID NO: 44), (Gly₃Ser)₂ (SEQ ID NO: 45), (Gly₃Ser)₁ (SEQ ID NO: 46), (Gly₂Ser)₂ (SEQ ID NO: 47) or (Gly₂Ser)₁, GGSGGGSGGSG (SEQ ID NO: 48), GGSGGGSGSG (SEQ ID NO: 49), or GGSGGGSG (SEQ ID NO: 50).

In particular embodiments, a linker region is (GGGGS)ₙ (SEQ ID NO: 37) wherein n is an integer including, 1, 2, 3, 4, 5, 6, 7, 8, 9, or more. In particular embodiments, the linker includes the Whitlow linker: GSTSGSGKPGSGEGSTKG (SEQ ID NO: 216; Whitlow et al., Protein Eng 6 (8): 989-95, 1993). In particular embodiments, the spacer is (EAAAK)ₙ (SEQ ID NO: 51) wherein n is an integer including 1, 2, 3, 4, 5, 6, 7, 8, 9, or more.

In some situations, flexible linkers may be incapable of maintaining a distance or positioning of a component needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

(III-E) Control Features Including Tag Cassettes, Transduction Markers, Selection Cassettes, and/or Suicide Switches. In particular embodiments, genetic constructs can encode one or more tag cassettes and/or transduction markers. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a genetic construct, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the genetic construct.

Tag cassettes that bind cognate binding molecules include, for example, His tag (HHHHHH; SEQ ID NO: 52), Flag tag (DYKDDDDK; SEQ ID NO: 53), Xpress tag (DLYDDDDK; SEQ ID NO: 54), Avi tag (GLNDIFEAQKIEWHE; SEQ ID NO: 55), Calmodulin tag (KRRWKKNFIAVSAANRFKKISSSGAL; SEQ ID NO: 56), Polyglutamate tag, HA tag (YPYDVPDYA; SEQ ID NO: 57), Myc tag (EQKLISEEDL; SEQ ID NO: 58), Strep tag (which refers the original STREP® tag (WRHPQFGG; SEQ ID NO: 59), STREP® tag II (WSHPQFEK SEQ ID NO: 60 (IBA Institut fur Bioanalytik, Germany); see, e.g., U.S. Pat. No. 7,981,632), Softag 1 (SLAELLNAGLGGS; SEQ ID NO: 61), Softag 3 (TQDPSRVG; SEQ ID NO: 62), and V5 tag (GKPIPNPLLGLDST; SEQ ID NO: 63).

Conjugate binding molecules that specifically bind tag cassette sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers may be selected from at least one of a truncated CD19 (tCD19 or CD19t; see Budde et al., Blood 122:1660, 2013); a truncated human EGFR (tEGFR or EGFRt; see Wang et al., Blood 118:1255, 2011); an ECD of human CD34; and/or RQR8 which combines target epitopes from CD34 (see Fehse et al, Mol. Therapy 1 (5 Pt 1); 448-456, 2000) and CD20 antigens (see Philip et al, Blood 124:1277-1278). In particular embodiments, cells are genetically modified to express a transduction marker. In particular embodiments, the transduction marker includes EGFRt or CD19t. In particular embodiments, the CD19t includes mouse CD19t (mCD19t).

In particular embodiments, a selection cassette provides for positive selection or negative selection of a desired cell population. Negative selection is when several cell types are removed, leaving the cell type of interest. Positive selection involves targeting the desired cell population to retain desired cells.

A selection cassette can encode proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cells. In particular embodiments, a positive selection cassette includes resistance genes to neomycin, hygromycin, ampicillin, puromycin, phleomycin, zeomycin, blasticidin, or viomycin. In particular embodiments, a selection cassette includes the DHFR (dihydrofolate reductase) gene or DHFR double mutant (DHFRdm) gene providing resistance to methotrexate (MTX), the MGMT P140K gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine) or other genes for detoxification with respect to some drugs. In particular embodiments, the selection agent includes neomycin, hygromycin, puromycin, phleomycin, zeomycin, blasticidin, viomycin, ampicillin, O⁶BG/BCNU, MTX, tetracycline, aminopterin, hypoxanthine, thymidine kinase, DHFR, Gln synthetase, or ADA.

In particular embodiments, negative selection cassettes include a gene for transformation of a substrate present in the culture medium into a toxic substance for the cell that expresses the gene. These molecules include detoxification genes of diptheria toxin (DTA) (Yagi et al., Anal Biochem. 214 (1): 77-86, 1993; Yanagawa et al., Transgenic Res. 8 (3): 215-221, 1999), the kinase thymidine gene of the Herpes virus (HSV TK) sensitive to the presence of ganciclovir or FIAU. The HPRT gene may also be used as a negative selection by addition of 6-thioguanine (6TG) into the medium, and for all positive and negative selections, a poly A transcription termination sequence from different origins, the most classical being derived from SV40 poly A, or a eukaryotic gene poly A (bovine growth hormone, rabbit β-globin, etc.).

In particular embodiments, genetic constructs can include a polynucleotide that encodes a self-cleaving polypeptide, wherein the polynucleotide encoding the self-cleaving polypeptide is located between the polynucleotide encoding the recombinant receptor and a polynucleotide encoding a pro-inflammatory cytokine (e.g., IL-18). In particular embodiments, genetic constructs can include a polynucleotide that encodes a self-cleaving polypeptide, wherein the polynucleotide encoding the self-cleaving polypeptide is located between the polynucleotide encoding the recombinant receptor or pro-inflammatory cytokine (e.g., IL-18) and a polynucleotide encoding a transduction marker (e.g., EGFRt or mCD19t). Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof. Further exemplary nucleic acid and amino acid sequences of 2A peptides are set forth in, for example, Kim et al. (PLOS One 6: e18556 (2011). In particular embodiments, cells are genetically modified to include a self-cleaving polypeptide. In particular embodiments, the self-cleaving polypeptide includes T2A. In particular embodiments, the self-cleaving polypeptide includes P2A.

Control features may be present in multiple copies in a genetic construct or can be expressed as distinct molecules with the use of a skipping element. For example, a genetic construct can have one, two, three, four or five tag cassettes and/or one, two, three, four, or five transduction markers could also be expressed. For example, embodiments can include a genetic construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247.

One advantage of including at least one control feature in a genetic construct is that cells expressing the genetic construct administered to a subject can be increased or depleted using the cognate binding molecule to a tag cassette. In certain embodiments, the present disclosure provides a method for depleting a modified cell expressing a genetic construct by using an antibody specific for the tag cassette, using a cognate binding molecule specific for the control feature, or by using a second modified cell expressing a recombinant receptor and having specificity for the control feature. Elimination of modified cells may be accomplished using depletion agents specific for a control feature. For example, if EGFRt is used, then an anti-EGFRt binding domain (e.g., antibody, scFv) fused to or conjugated to a cell-toxic reagent (such as a toxin, radiometal) may be used, or an anti-EGFRt/anti-CD3 bispecific scFv, or an anti-EGFRt CAR T cell may be used.

In particular embodiments, a polynucleotide encoding an iCaspase9 construct (iCasp9) may be inserted into a genetic construct as a suicide switch.

In certain embodiments, modified cells expressing a genetic construct may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature can be, e.g., more readily identified, isolated, sorted, induced to proliferate, tracked, and/or eliminated as compared to a modified cell without a tag cassette.

(IV) Characterization of Genetically Engineered Cells. In particular embodiments, the engineered cells can be assessed for expression of the genetic construct (e.g., IL-18 and/or CAR). In particular embodiments, at least 50%, at least 55%, at least 60%, at least 65%, at least 70%, at least 75%, at least 80%, at least 85%, at least 90%, at least 95%, or at least 99% of the engineered cells express a detectable level of the genetic construct.

Surface protein expression can be determined by flow cytometry using methods known in the art. By labeling a population of cells with an element that targets the desired cell surface marker (e.g., an antibody) and is tagged with a fluorescent molecule, flow cytometry can be used to quantify the portion of the population that is positive for the surface marker, as well as the level of surface marker expression.

Genomic incorporation of a genetic construct within engineered cells can be determined by digital droplet PCR (ddPCR). Digital PCR enables quantification of DNA concentration in a sample. Digital PCR is performed by fractionating a mixture of a PCR reaction (e.g., containing a sample of nucleic acid molecules and copies of a PCR probe) such that some fractions contain no PCR probe copy, while other fractions contain one or more PCR probe copies. A PCR amplification of the fractions is performed and the fractions are analyzed for a PCR reaction. A fraction containing one or more probes and one or more target DNA molecules yields a positive end-point, while a fraction containing no PCR probe yields a negative end-point. The fraction of positive reactions is then fitted to a Poisson distribution to determine the absolute copy number of target DNA molecules per given volume of the unfractionated sample (i.e., copies per microliter of sample) (see Hindson, B. et al., (2011) Anal Chem. 83:8604-8610). Digital droplet PCR is a variation of digital PCR wherein a sample of nucleic acids is fractionated into droplets using a water-oil emulsion. PCR amplification is performed on the droplets collectively, whereupon a fluidics system is used to separate the droplets and provide analysis of each individual droplet. For one skilled in the art, ddPCR is used to provide an absolute quantification of DNA in a sample, to perform a copy number variation analysis, or to assess efficiency of genomic edits.

Engineered cells can also be assessed for cytokine-independent growth. Engineered cells are expected to grow in the presence of stimulatory cytokines (e.g., IL-2, IL-7). Growth in the absence of cytokines is an indicator of tumorigenic potential. In particular embodiments, engineered cells are grown for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days in either the presence or in the absence of one or more stimulatory cytokines (e.g., IL-2, IL-7). In particular embodiments, proliferation is assessed by cell count and viability using conventional methods (e.g., flow cytometry, microscopy, optical density, metabolic activity). In particular embodiments, proliferation is assessed starting on day 1, day 2, day 3, day 4, day 5, day 6. In particular embodiments, proliferation is assessed every 1 day, every 2 days, every 3 days, every 4 days, every 5 days, every 6 days, every 7 days, or every 8 days. In particular embodiments, growth in the absence of cytokines is assessed at the end of a growth period. In some particular embodiments, engineered cells with no growth in the absence of cytokines is defined as lacking tumorigenic potential. In particular embodiments, no growth is defined as an expansion of the population that is less than 0.1, 0.2, 0.3, 0.4, 0.5, 0.6, 0.7, 0.8, 0.9, 1.0, 1.1, 1.2, 1.3, 1.4, or 1.5-fold between the end of the growth period relative to the beginning of the growth period. In particular embodiments, the engineered cells do not proliferate in the absence of cytokine stimulation, growth factor stimulation, or antigen stimulation.

(V) Cell Activating Culture Conditions. Cell populations can be incubated in a culture-initiating composition to expand cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

In particular embodiments, the cell population can be incubated in the culture-initiating composition before or after genetic engineering the cell populations. In particular embodiments, the incubation can be carried out for 1 day to 6 days, 1 day to 5 days, 1 day to 4 days, 1 day to 3 days, 1 day to 2 days, or 1 day before genetically engineering the cell populations. In particular embodiments, the incubation can be carried out for 1 day to 6 days, 1 day to 5 days, 1 day to 4 days, 1 day to 3 days, 1 day to 2 days, or 1 day after genetically engineering the cell populations. In particular embodiments, the incubation can be carried out at the same time as genetically engineering the cell populations.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35 (9): 651-660, Terakura et al. (2012) Blood. 1:72-82, and/or Wang et al. (2012) J Immunother. 35 (9): 689-701.

Exemplary culture media for culturing T cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10-4-0.75×10-4 M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T cell culture media are also commercially available from Hyclone (Logan, UT). Additional T cell activating components that can be added to such culture media are described in more detail below.

In some embodiments, the T cells are expanded by adding to the culture-initiating composition feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can include gamma-irradiated PBMC feeder cells. In some embodiments, the PBMC are irradiated with gamma rays in the range of 3000 to 3600 rads to prevent cell division. In some aspects, the feeder cells are added to culture medium prior to the addition of the populations of T cells.

Optionally, the incubation may further include adding non-dividing EBV-transformed lymphoblastoid cells (LCL) as feeder cells. LCL can be irradiated with gamma rays in the range of 6000 to 10,000 rads. The LCL feeder cells in some aspects is provided in any suitable amount, such as a ratio of LCL feeder cells to initial T lymphocytes of at least 10:1.

In some embodiments, the stimulating conditions include temperature suitable for the growth of human T lymphocytes, for example, at least 25 °C, at least 30 °C, or 37 °C.

The activating culture conditions for T cells include conditions whereby T cells of the culture-initiating composition proliferate or expand. T cell activating conditions can include one or more cytokines, for example, interleukin (IL)-2, IL-7, IL-15 and/or IL-21. IL-2 can be included at a range of 10-100 ng/ml (e.g., 40, 50, or 60 ng/ml). IL-7, IL-15, and/or IL-21 can be individually included at a range of 0.1-50 ng/ml (e.g., 5, 10, or 15 ng/ml). Particular embodiments utilize IL-2 at 50 ng/ml. Particular embodiments utilize, IL-7, IL-15 and IL-21 individually included at 10 ng/ml.

In particular embodiments, T cell activating culture conditions can include T cell stimulating epitopes. T cell stimulating epitopes include CD3, CD27, CD2, CD4, CD5, CD7, CD8, CD28, CD30, CD40, CD56, CD83, CD90, CD95, 4-1BB (CD 137), B7-H3, CTLA-4, Frizzled-1 (FZD1), FZD2, FZD3, FZD4, FZD5, FZD6, FZD7, FZD8, FZD9, FZD10, HVEM, ICOS, IL-1R, LAT, LFA-1, LIGHT, MHCI, MHCII, NKG2D, OX40, ROR2 and RTK.

CD3 is a primary signal transduction element of T cell receptors. As indicated previously, CD3 is expressed on all mature T cells. In particular embodiments, the CD3 stimulating molecule (i.e., CD3 binding domain) can be derived from the OKT3 antibody (see U.S. Pat. Nos. 5,929,212; 4,361,549; ATCC® CRL-8001™; and Arakawa et al., J. Biochem. 120, 657-662 (1996)), the 20G6-F3 antibody, the 4B4-D7 antibody, the 4E7-C9, or the 18F5-H10 antibody.

In particular embodiments, CD3 stimulating molecules can be included within culture media at a concentration of at least 0.25 or 0.5 ng/ml or at a concentration of 2.5-10 μg/ml. Particular embodiments utilize a CD3 stimulating molecule (e.g., OKT3) at 5 μg/ml.

In particular embodiments, activating molecules associated with avi-tags can be biotinylated and bound to streptavidin beads. This approach can be used to create, for example, a removable T cell epitope stimulating activation system.

An exemplary binding domain for CD28 can include or be derived from TGN1412, CD80, CD86 or the 9D7 antibody. Additional antibodies that bind CD28 include 9.3, KOLT-2, 15E8, 248.23.2, EX5.3D10, and CD28.3 (deposited as a synthetic single chain Fv construct under GenBank Accession No. AF451974.1; see also Vanhove et al., BLOOD, 15 Jul. 2003, Vol. 102, No. 2, pages 564-570). Further, 1YJD provides a crystal structure of human CD28 in complex with the Fab fragment of a mitogenic antibody (5.11A1). In particular embodiments, antibodies that do not compete with 9D7 are selected.

4-1BB binding domains can be derived from LOB12, IgG2a, LOB12.3, or IgG1 as described in Taraban et al. Eur J Immunol. 2002 December; 32 (12): 3617-27. In particular embodiments a 4-1BB binding domain is derived from a monoclonal antibody described in U.S. Pat. No. 9,382,328. Additional 4-1BB binding domains are described in U.S. Pat. Nos. 6,569,997, 6,303,121, and Mittler et al. Immunol Res. 2004; 29 (1-3): 197-208.

OX40 (CD134) and/or ICOS activation may also be used. OX40 binding domains are described in US20100196359, US20150307617, WO 2015/153513, WO2013/038191 and Melero et al. Clin Cancer Res. 2013 Mar. 1; 19 (5): 1044-53. Exemplary binding domains that can bind and activate ICOS are described in e.g., US20080279851 and Deng et al. Hybrid Hybridomics. 2004 June; 23 (3): 176-82.

When in soluble form, T-cell activating agents can be coupled with another molecule, such as polyethylene glycol (PEG) molecule. Any suitable PEG molecule can be used. Typically, PEG molecules up to a molecular weight of 1000 Da are soluble in water or culture media. In some cases, such PEG based reagent can be prepared using commercially available activated PEG molecules (for example, PEG-NHS derivatives available from NOF North America Corporation, Irvine, Calif., USA, or activated PEG derivatives available from Creative PEGWorks, Chapel Hills, N.C., USA).

In particular embodiments, cell stimulating agents are immobilized on a solid phase within the culture media. In particular embodiments, the solid phase is a surface of the culture vessel (e.g., bag, cell culture plate, chamber, chromatography column, cross-linked gel, cross-linked polymer, column, culture dish, hollow fiber, microtiter plate, silica-coated glass plate, tube, tubing set, well, vial, other structure or container for culture or cultivation of cells).

In particular embodiments, a solid phase can be added to a culture media. Such solid phases can include, for example, beads, hollow fibers, resins, membranes, and polymers.

Exemplary beads include magnetic beads, polymeric beads, and resin beads (e.g., Strep-Tactin® Sepharose, Strep-Tactin® Superflow, and Strep-Tactin® MacroPrep IBA GmbH, Gottingen)). Anti-CD3/anti-CD28 beads are commercially available reagents for T cell expansion (Invitrogen). These beads are uniform, 4.5 μm superparamagnetic, sterile, non-pyrogenic polystyrene beads coated with a mixture of affinity purified monoclonal antibodies against the CD3 and CD28 cell surface molecules on human T cells. Hollow fibers are available from TerumoBCT Inc. (Lakewood, Colo., USA). Resins include metal affinity chromatography (IMAC) resins (e.g., TALON® resins (Westburg, Leusden)). Membranes include paper as well as the membrane substrate of a chromatography matrix (e.g., a nitrocellulose membrane or a polyvinylidene difluoride (PVDF) membrane).

Exemplary polymers include polysaccharides, such as polysaccharide matrices. Such matrices include agarose gels (e.g., Superflow™ agarose or a Sepharose® material such as Superflow™ Sepharose® that are commercially available in different bead and pore sizes) or a gel of crosslinked dextran(s). A further illustrative example is a particulate cross-linked agarose matrix, to which dextran is covalently bonded, that is commercially available (in various bead sizes and with various pore sizes) as Sephadex® or Superdex®, both available from GE Healthcare.

Synthetic polymers that may be used include polyacrylamide, polymethacrylate, a co-polymer of polysaccharide and agarose (e.g. a polyacrylamide/agarose composite) or a polysaccharide and N,N′-methylenebisacrylamide. An example of a copolymer of a dextran and N,N′-methylenebisacrylamide is the Sephacryl® (Pharmacia Fine Chemicals, Inc., Piscataway, NJ) series of materials.

Particular embodiments may utilize silica particles coupled to a synthetic or to a natural polymer, such as polysaccharide grafted silica, polyvinylpyrrolidone grafted silica, polyethylene oxide grafted silica, poly(2-hydroxyethylaspartamide) silica and poly(N-isopropylacrylamide) grafted silica.

Cell activating agents can be immobilized to solid phases through covalent bonds or can be reversibly immobilized through non-covalent attachments.

In particular embodiments, a T-cell activating culture media includes a FACS-sorted T cell population cultured within RPMI with HEPES, 5-15% human serum, 1-3% L-Glutamine, 0.5-1.5% Pen/strep, 0.25×10-4-0.75×10⁻⁴ M β-MercaptoEthanol, with IL-7, IL-15 and IL-21 individually included at 5-15 (e.g., 10) ng/ml. The culture is carried out on a flat-bottom well plate with 0.1-0.5×10e6 plated cells/well. On Day 3 post activation cells are transferred to a TC-treated plate.

In particular embodiments, a T-cell activating culture media includes a FACS-sorted CD8+T population cultured within RPMI with HEPES, 10% human serum, 2% L-Glutamine, 1% Pen/strep, 0.5×10⁻⁴ M β-MercaptoEthanol, with IL-7, IL-15 and IL-21 individually included at 5-15 (e.g., 10) ng/ml. The culture is carried out on a flat-bottom non-tissue culture (TC)-treated 96/48-well plate with 0.1-0.5×10e6 plated cells/well. On Day 3 post activation cells are transferred to TC-treated plate.

Culture conditions for HSC/HSP can include expansion with a Notch agonist (see, e.g., U.S. Pat. Nos. 7,399,633; 5,780,300; 5,648,464; 5,849,869; and 5,856,441 and growth factors present in the culture condition as follows: 25-300 ng/ml SCF, 25-300 ng/ml Flt-3L, 25-100 ng/ml TPO, 25-100 ng/ml IL-6 and 10 ng/ml IL-3. In more specific embodiments, 50, 100, or 200 ng/ml SCF; 50, 100, or 200 ng/ml of Flt-3L; 50 or 100 ng/ml TPO; 50 or 100 ng/ml IL-6; and 10 ng/ml IL-3 can be used.

(VI) Ex Vivo Manufactured Cell Formulations. In particular embodiments, genetically modified cells can be harvested from a culture medium and washed and concentrated into a carrier in a therapeutically-effective amount. Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 mls or less, 250 mls or less or 100 mls or less. Hence the density of administered cells is typically greater than 10⁴ cells/ml, 10⁷ cells/ml or 10⁸ cells/ml.

As indicated, formulations can include at least one genetically modified cell type (e.g., modified T cells, NK cells, or stem cells). Formulations can include different types of genetically-modified cells (e.g., T cells, NK cells, and/or stem cells in combination).

Different types of genetically-modified cells or cell subsets (e.g., modified T cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different recombinant receptor components. If only two of the cell types are combined or only 2 combinations of expressed recombinant receptor components are included within a formulation, the ratio can include any 2-number combination that can be created from the 3 number combinations provided above. In embodiments, the combined cell populations are tested for efficacy and/or cell proliferation in vitro, in vivo and/or ex vivo, and the ratio of cells that provides for efficacy and/or proliferation of cells is selected. Particular embodiments include a 1:1 ratio of CD4 T cells and CD8 T cells.

The cell-based formulations disclosed herein can be prepared for administration by, e.g., injection, infusion, perfusion, or lavage. The formulations can further be formulated for bone marrow, intravenous, intradermal, intraarterial, intranodal, intralymphatic, intraperitoneal, intralesional, intratumoral, intravesicular, and/or subcutaneous injection.

(VII) Targeted Viral Vectors & Nanoparticles. Targeted viral vectors and/or nanoparticles can also be used to genetically-modify immune cells in vivo or ex vivo. Viral vectors that can be used to deliver genetic constructs described herein to cells are described elsewhere herein, and numerous targeted (e.g., pseudotyped) viral vectors are known in the art.

Exemplary cell-targeted nanoparticles include a cell targeting ligand (e.g., CD3, CD4, CD8, CD34) on the surface of the nanoparticle wherein the cell targeting ligand results in selective uptake of the nanoparticle by a selected cell type. The nanoparticle then delivers gene modifying components that result in expression of the recombinant receptor and pro-inflammatory cytokine.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy. In particular embodiments, the nanoparticles can be those described in WO2014153114, WO2017181110, and WO201822672.

Therapeutically effective amounts of vectors and/or nanoparticles within formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

(VIII) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with formulations disclosed herein. Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer's development or progression. An immunogenic formulation can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a STEAP1-expressing cancer. In particular embodiments, prophylactic treatments reduce, delay, or prevent metastasis from a primary a cancer tumor site from occurring.

A “therapeutic treatment” includes a treatment administered to a subject who displays symptoms or signs of a cancer and is administered to the subject for the purpose of diminishing or eliminating those signs or symptoms. The therapeutic treatment can reduce, control, or eliminate the presence or activity of the cancer and/or reduce, control, or eliminate side effects of the cancer.

Function as an effective amount, prophylactic treatment, or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment. In particular embodiments, therapeutically effective amounts provide anti-cancer effects in low antigen density conditions.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

In particular embodiments, therapeutically effective amounts induce an immune response. The immune response can be against a STEAP1-expressing cancer cell.

The term “STEAP1-expressing cell” or “STEAP1-positive cell” refers to a cell that expresses STEAP1 on its surface. The term “STEAP1-positive cancer cell” refers to a cancer cell that expresses STEAP1 on its surface. In some embodiments, expression of STEAP1 on the cell surface is determined, for example, using antibodies to STEAP1 in a method such as immunohistochemistry, FACS, etc. Alternatively, STEAP1 mRNA expression is considered to correlate to STEAP1 expression on the cell surface and can be determined by, for example, in situ hybridization and/or RT-PCR (including quantitative RT-PCR).

Examples of STEAP1-related disorders that can be treated with genetic constructs disclosed herein include prostate cancer (e.g., castration-resistant prostate cancer), the Ewing family of tumors (including Ewing's sarcoma), bladder cancer, breast cancer, ovarian cancer, colon cancer, lung cancer, and kidney cancer.

Genetic constructs disclosed herein can be used to treat subjects having cancers with low antigen density conditions, as described above. Certain examples include assessing a subject's cancer for STEAP1 antigen expression levels and selecting a recombinant receptor of the current disclosure to treat the subject based on the presence of low antigen density conditions.

The recombinant receptor disclosed herein are not limited to treating subjects having cancers with low antigen density conditions, and can also be used in subjects having cancers with high antigen density conditions. High antigen density conditions include those with more than 50,000 STEAP1 molecules per diseased cell; more than 60,000 STEAP1 molecules per diseased cell; more than 70,000 STEAP1 molecules per diseased cell; more than 80,000 STEAP1 molecules per diseased cell; more than 90,000 STEAP1 molecules per diseased cell; or more than 100,000 STEAP1 molecules per diseased cell.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of cancer, previous or concurrent therapeutic interventions, idiopathy of the subject and route of administration.

Therapeutically effective amounts of cell-based formulations can include 104 to 10⁹ cells/kg body weight, or 103 to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of vectors and/or nanoparticles within formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intrathecal, intratumoral, intravesicular, and/or subcutaneous.

In certain embodiments, cells are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

In certain embodiments, formulations including immune effector cells genetically modified with genetic constructs disclosed herein may be administered in conjunction with any number of chemotherapeutic agents. Examples of chemotherapeutic agents include alkylating agents; alkyl sulfonates; aziridines; ethylenimines and methylamelamines; nitrogen mustards; nitrosureas; antibiotics; anti-metabolites); folic acid analogues; purine analogs; pyrimidine analogs; androgens; anti-adrenals; folic acid replenisher; taxoids; platinum analogs I; topoisomerase inhibitor; retinoic acid derivatives; and pharmaceutically acceptable salts, acids or derivatives of any of the above. Also included in this definition are anti-hormonal agents that act to regulate or inhibit hormone action on tumors such as anti-estrogens and anti-androgens and pharmaceutically acceptable salts, acids or derivatives of any of the above. Combinations of chemotherapeutic agents are also administered where appropriate, including, CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In other embodiments, the chemotherapeutic agent is administered at least 1 month before administering the cell or nucleic acid. In some embodiments, the methods further include administering two or more chemotherapeutic agents.

A variety of additional therapeutic agents may be used in conjunction with the formulations described herein. For example, potentially useful additional therapeutic agents include PD-1 inhibitors such as nivolumab (Opdivo®), pembrolizumab (Keytruda®), pembrolizumab, pidilizumab, and atezolizumab, and CTLA-4 inhibitors, such as ipilimumab (Yervoy®).

Additional therapeutic agents suitable for use in combination with the disclosure include abiraterone acetate, apalutamide, bicalutamide, cabazitaxel, casodex (bicalutamide), degarelix, docetaxel, enzalutamide, Erleada® (apalutamide), flutamide, goserelin acetate, Jevtana® (cabazitaxel), leuprolide acetate, Lupron® (leuprolide acetate), Lupron Depot (leuprolide acetate), Lupron Depot-Ped (leuprolide acetate), mitoxantrone hydrochloride, Nilandron® (nilutamide), nilutamide, Provenge® (Sipuleucel-T), radium 223 di chloride, sipuleucel-T, taxotere (docetaxel), Viadur (leuprolide acetate), Xofigo (radium 223 dichloride), Xtandi (enzalutamide), Zoladex (goserelin acetate), or Zytiga (abiraterone acetate).

In additional embodiments, the formulations described herein can be administered with an anti-inflammatory agent. Anti-inflammatory agents or drugs include steroids and glucocorticoids, nonsteroidal anti-inflammatory drugs (NSAIDS). Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofm) and intramuscular) and minocycline.

In certain embodiments, the formulations described herein are administered in conjunction with another cytokine (e.g., in addition to the pro-inflammatory cytokine expressed by the genetic construct). “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-II, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

(IX) Reference Levels Derived from Control Populations. Obtained values for parameters associated with a therapy described herein can be compared to a reference level derived from a control population, and this comparison can indicate whether a therapy described herein is effective for a subject in need thereof. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 60-65 years) and cancer status. In particular embodiments, a normal control population includes individuals that are age-matched to a test subject and do not have cancer. In particular embodiments, age-matched includes, e.g., 0-10 years old; 30-40 years old, 60-65 years old, 70-85 years old, etc., as is clinically relevant under the circumstances. In particular embodiments, a control population can include those that have a STEAP1-related disorder and have not been administered a therapeutically effective amount.

In particular embodiments, the relevant reference level for values of a particular parameter associated with a therapy described herein is obtained based on the value of a particular corresponding parameter associated with a therapy in a control population to determine whether a therapy disclosed herein has been therapeutically effective for a subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

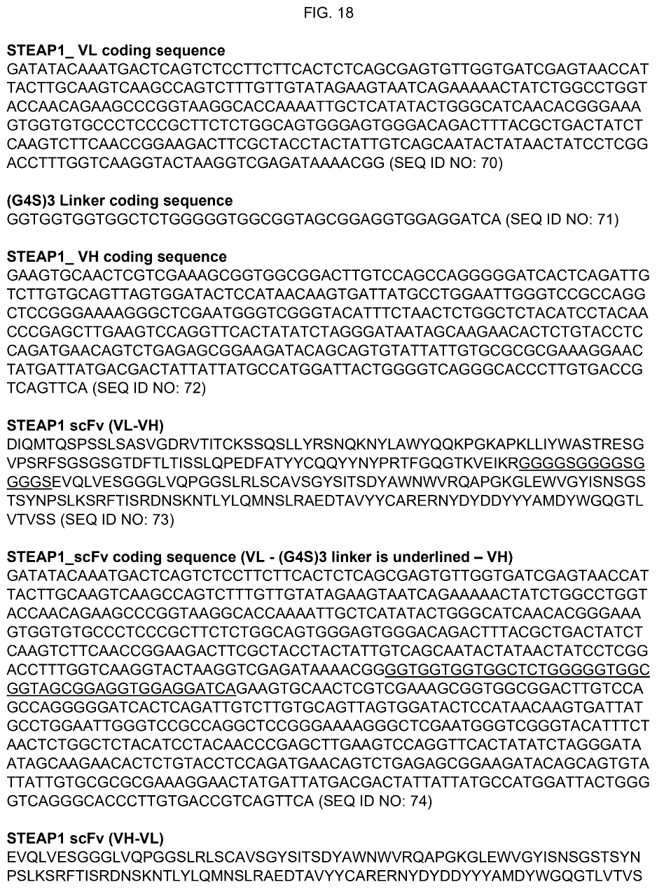

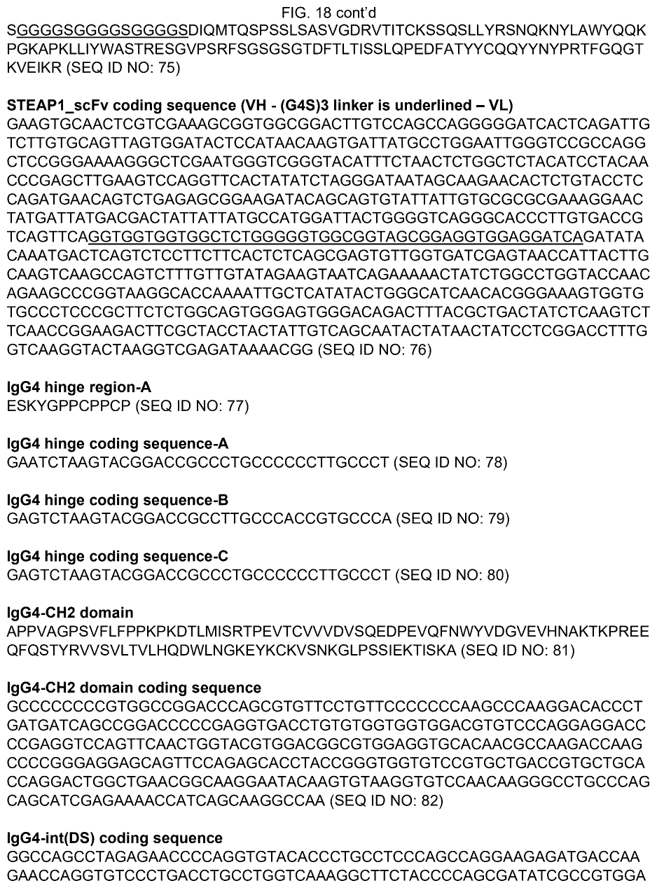

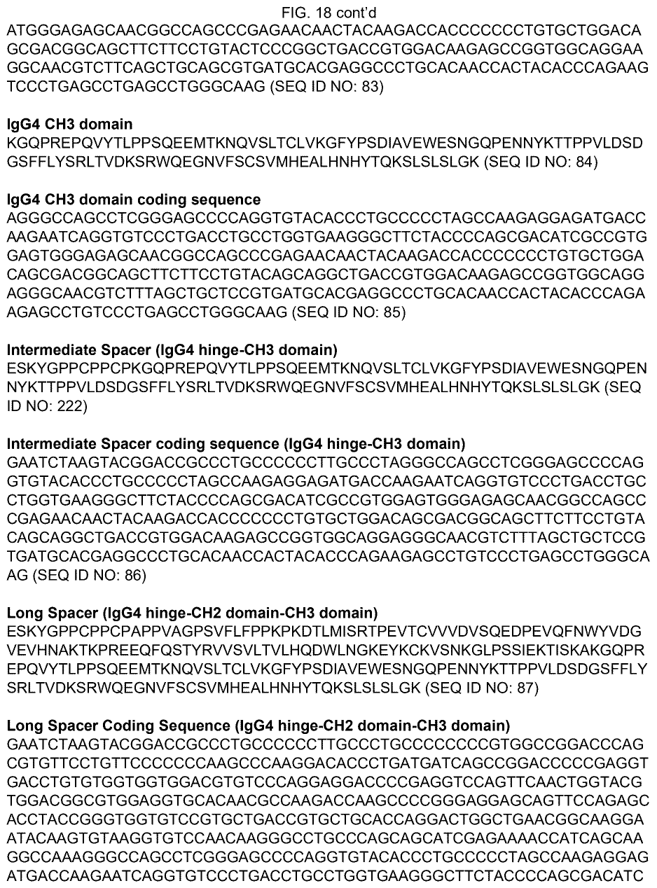

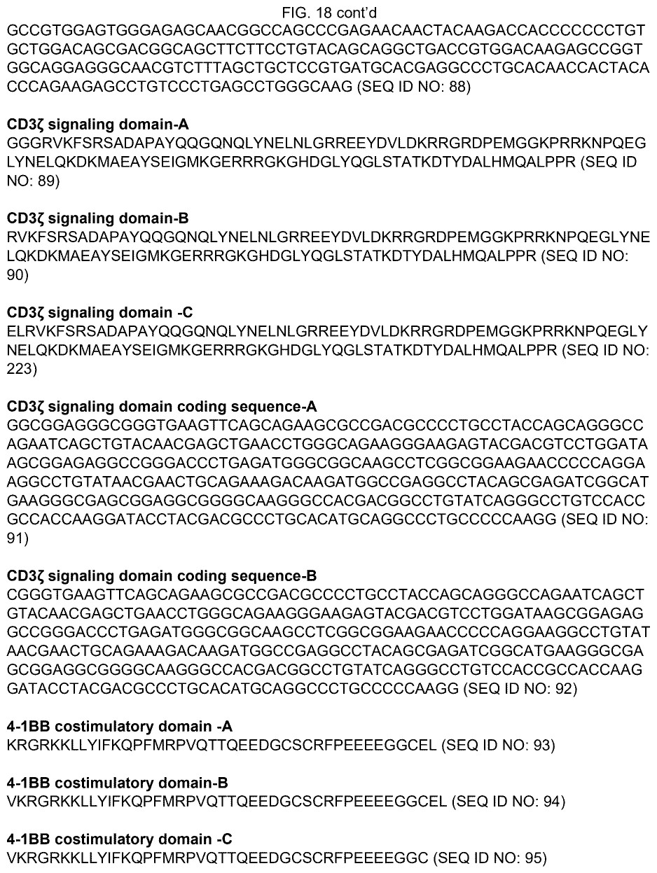

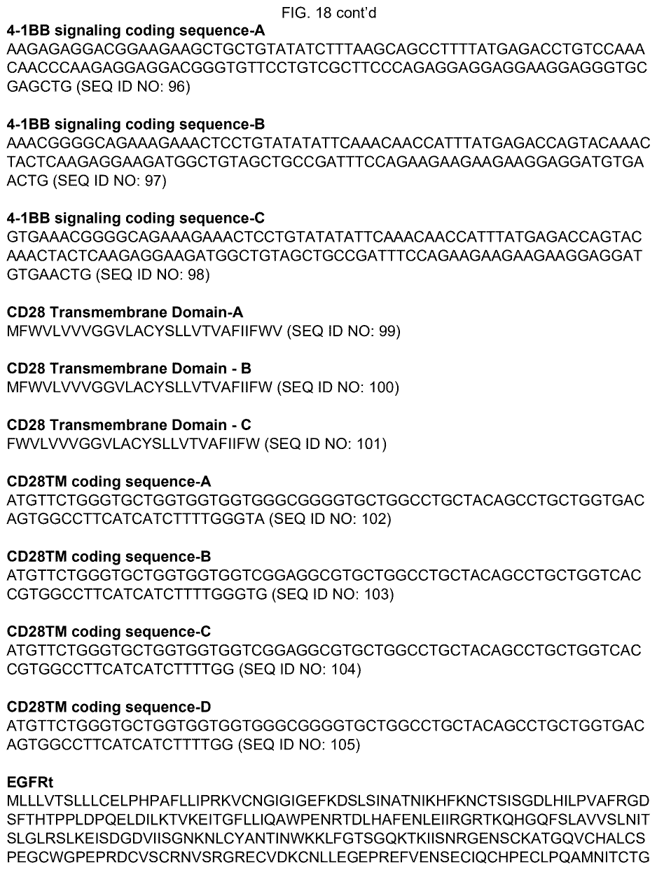

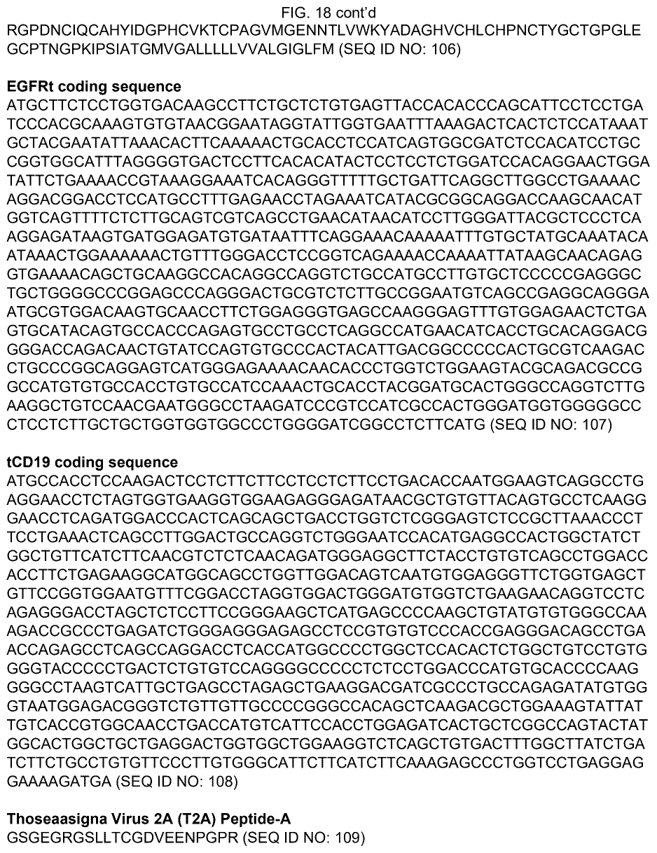

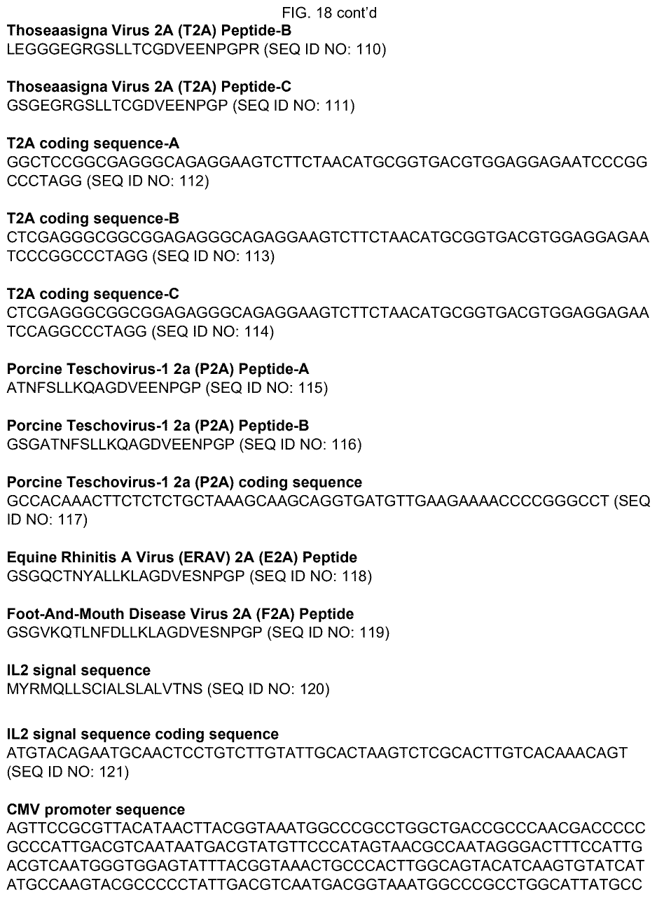

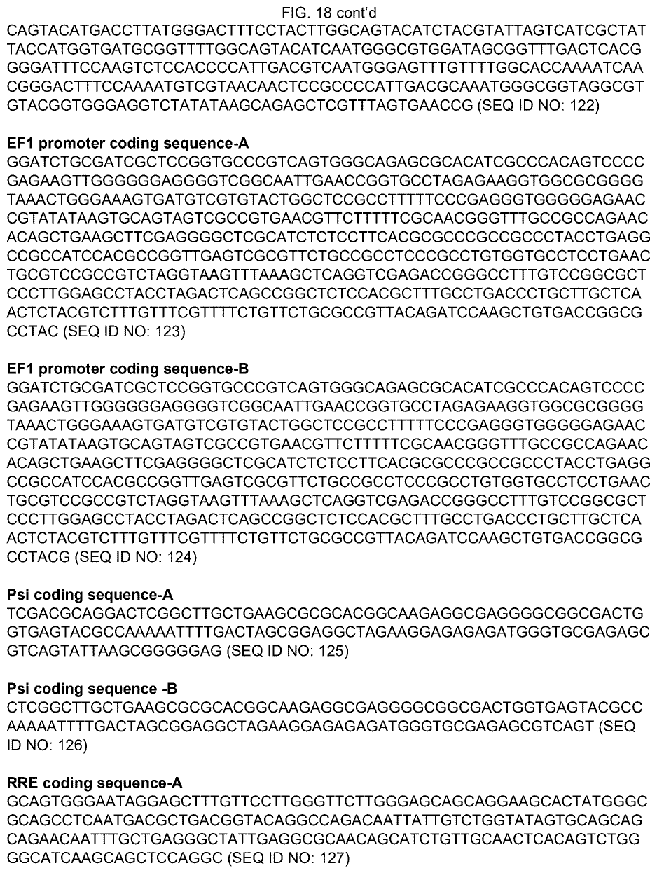

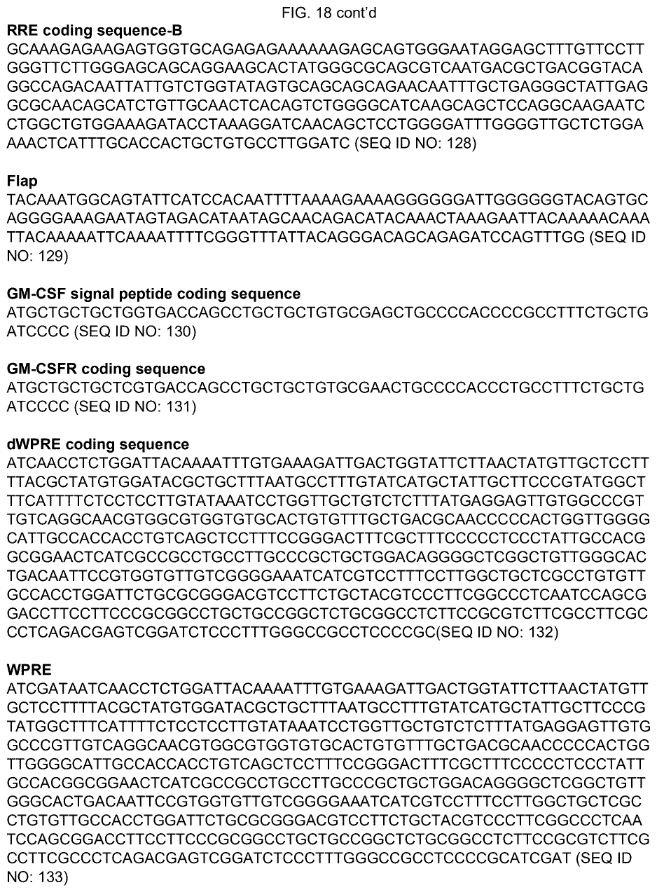

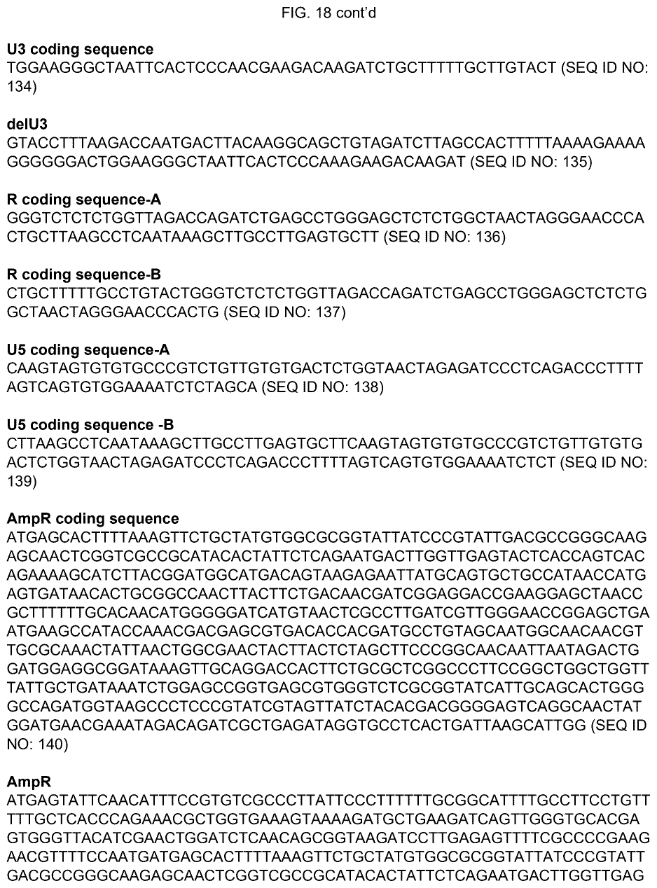

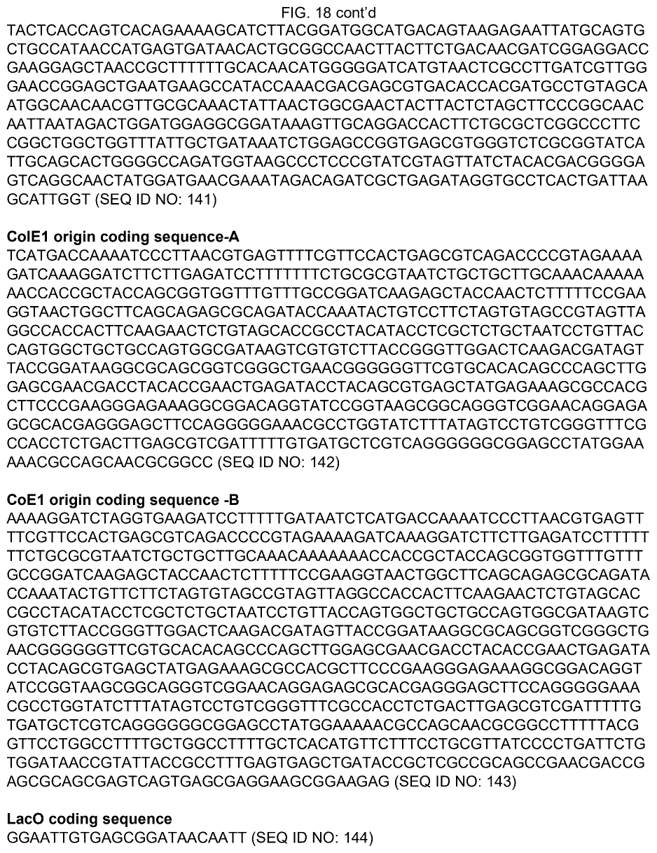

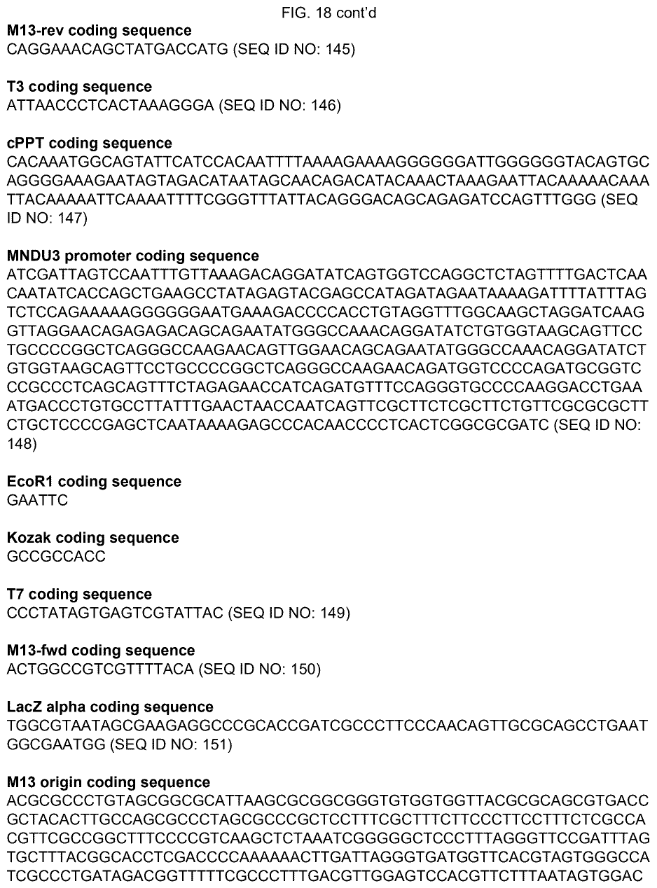

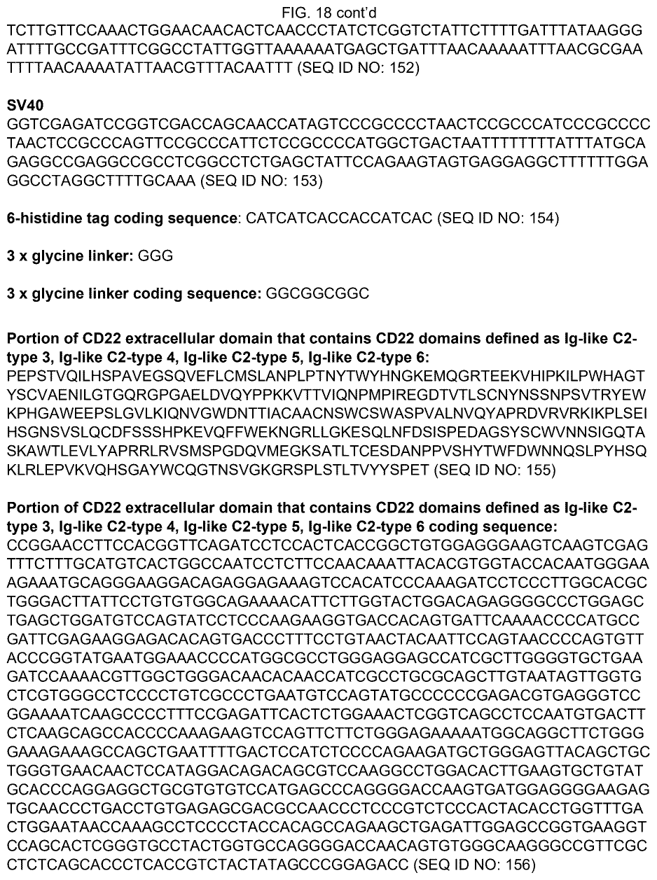

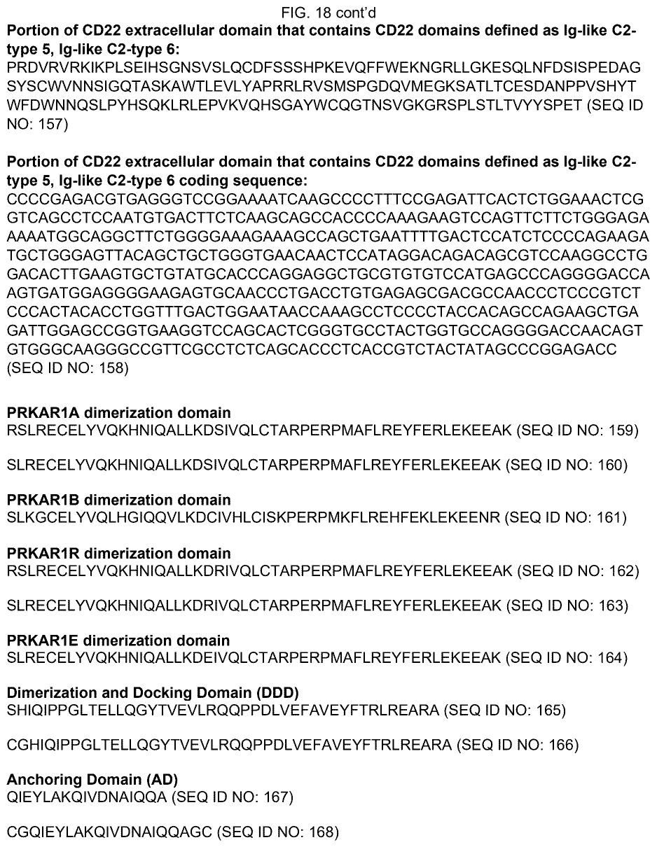

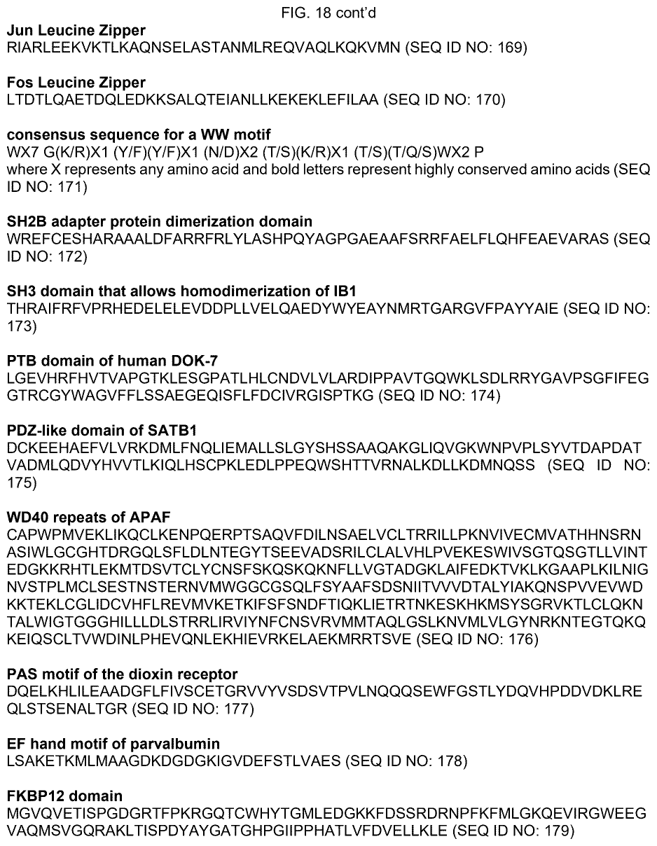

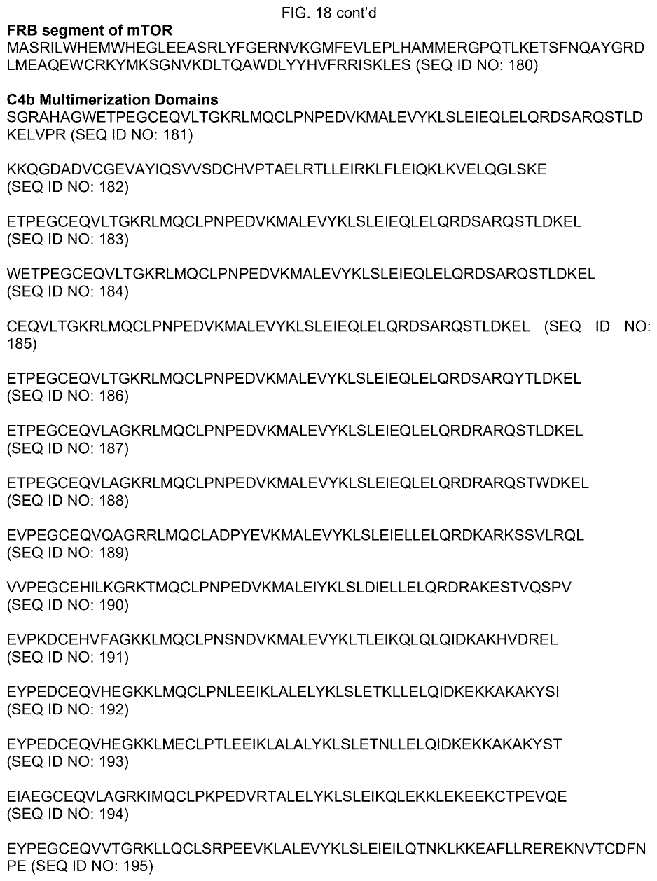

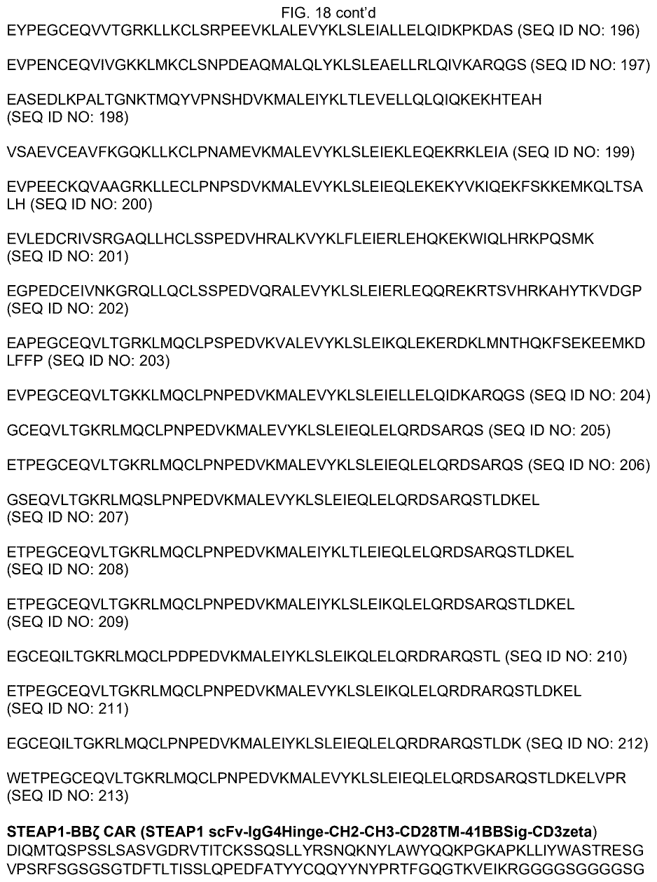

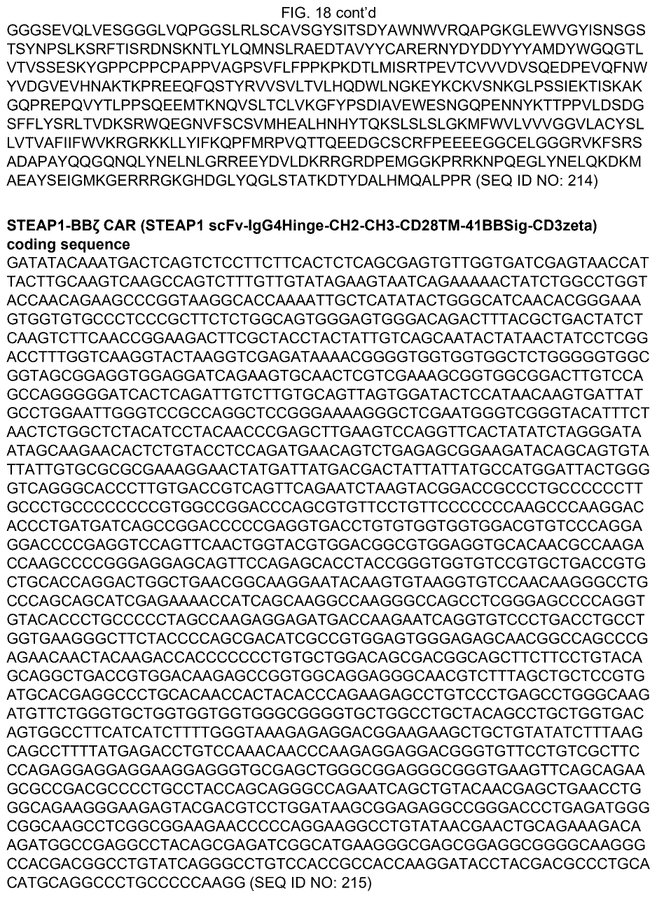

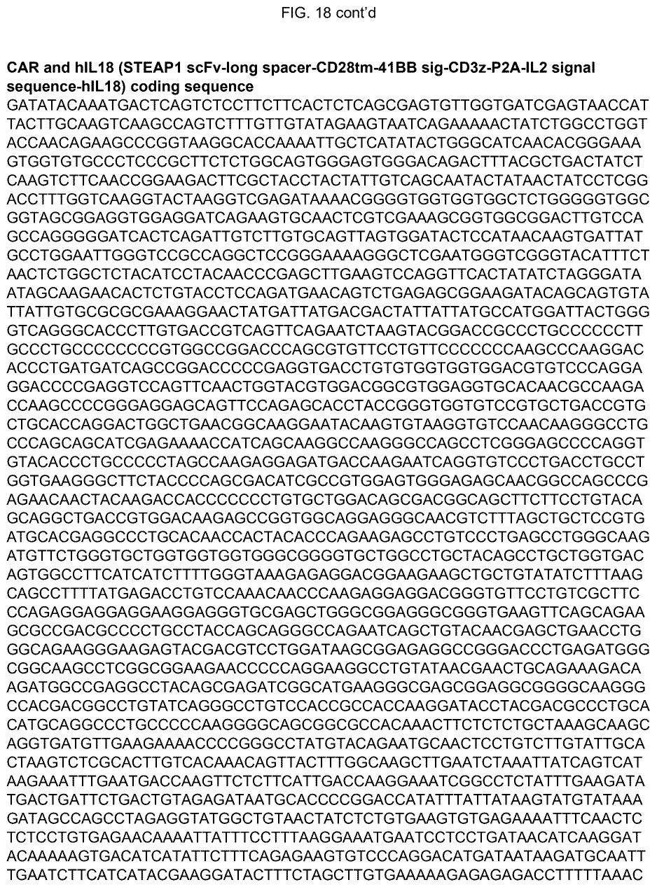

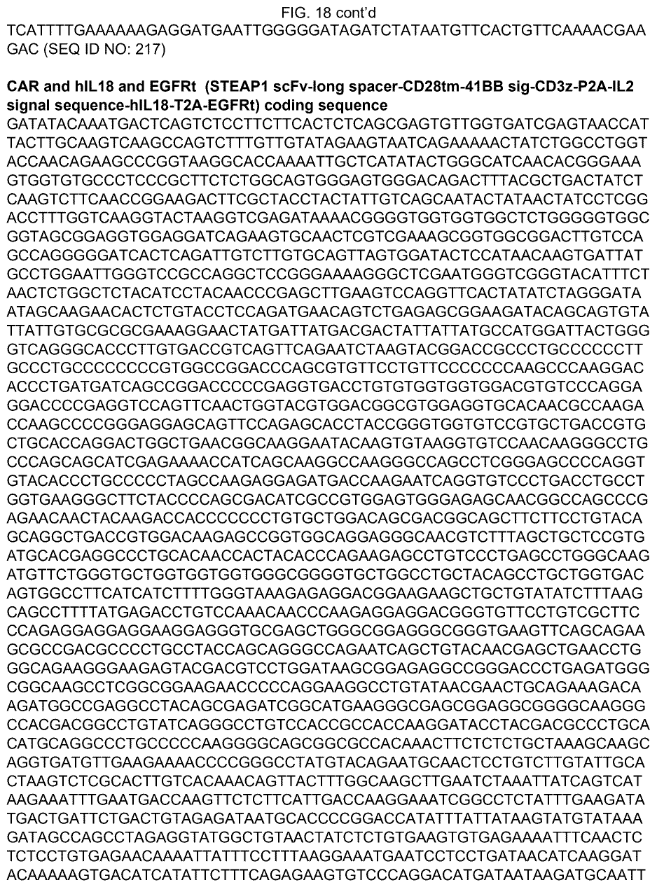

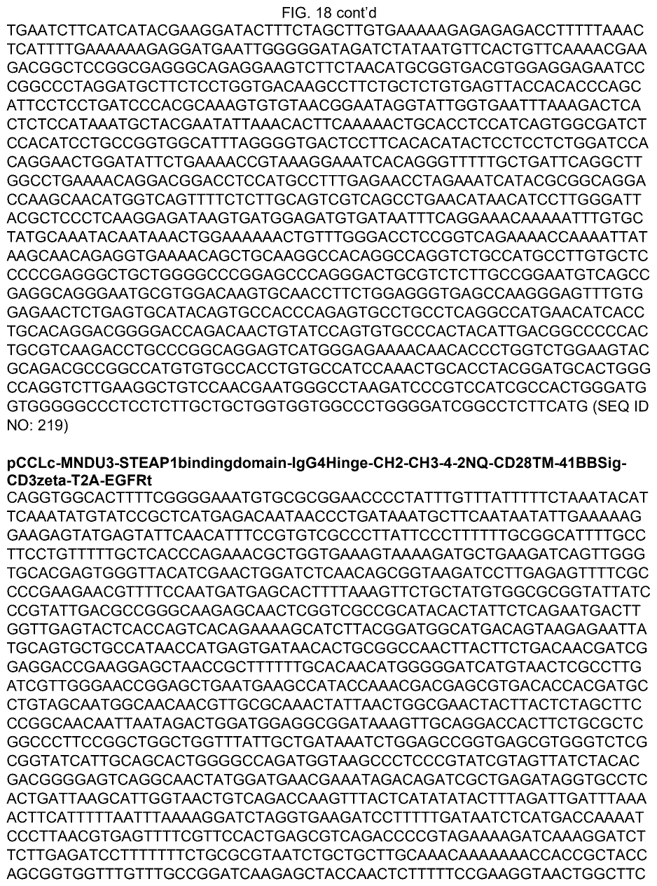

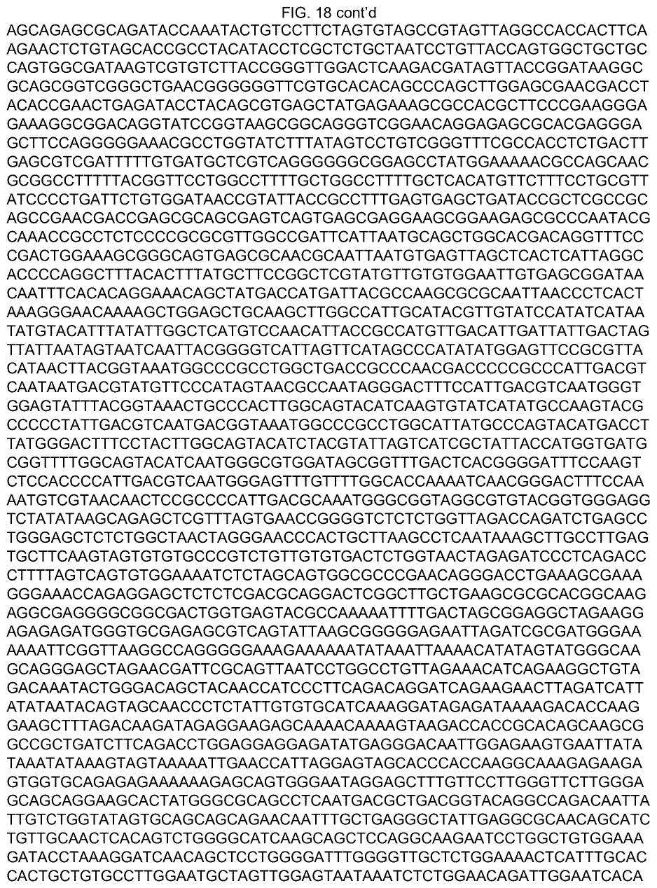

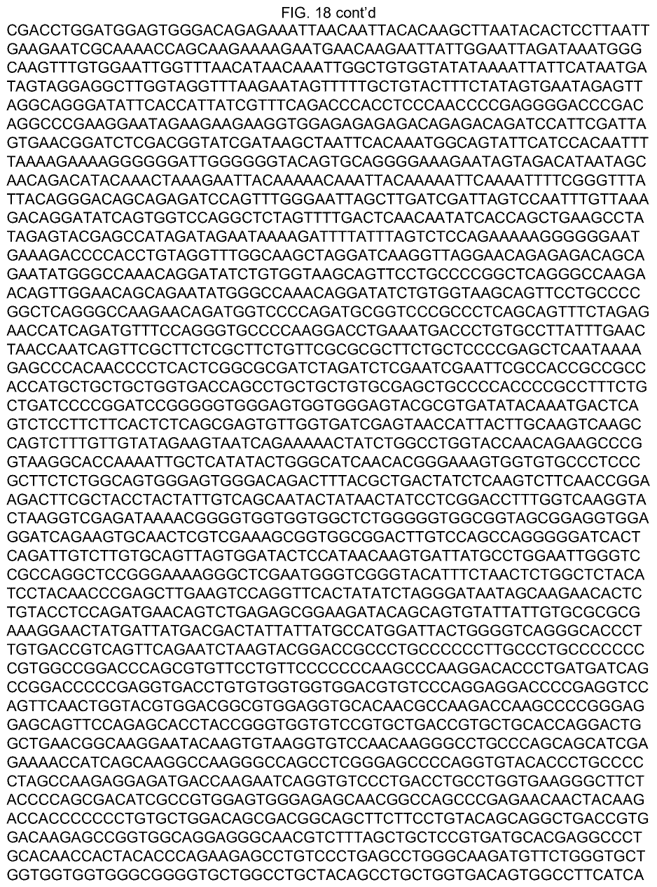

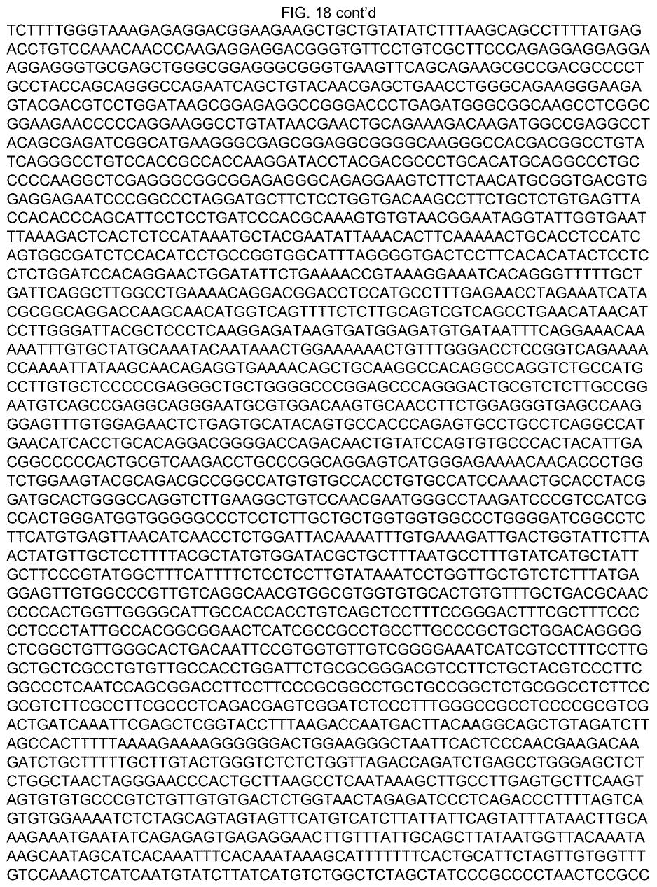

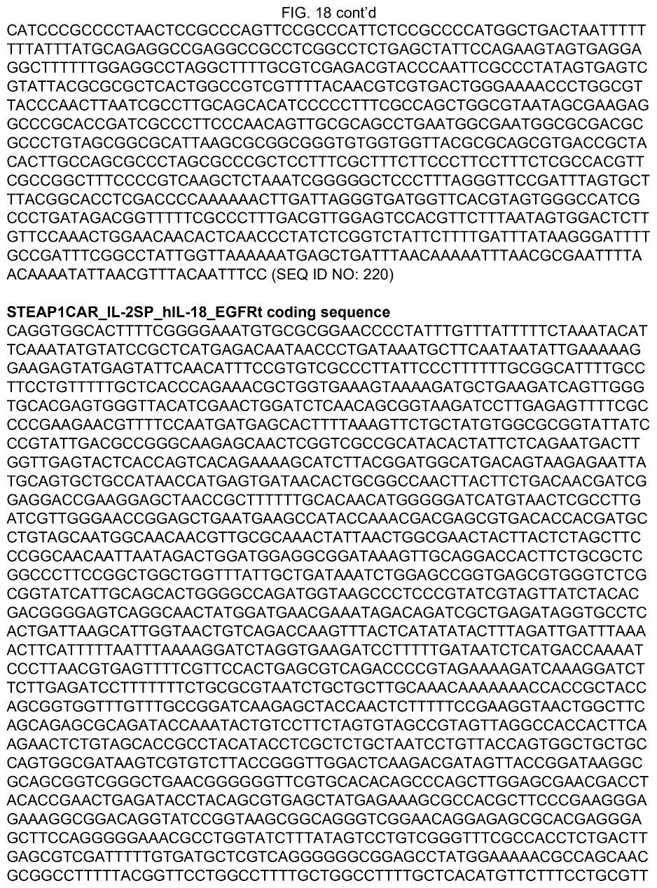

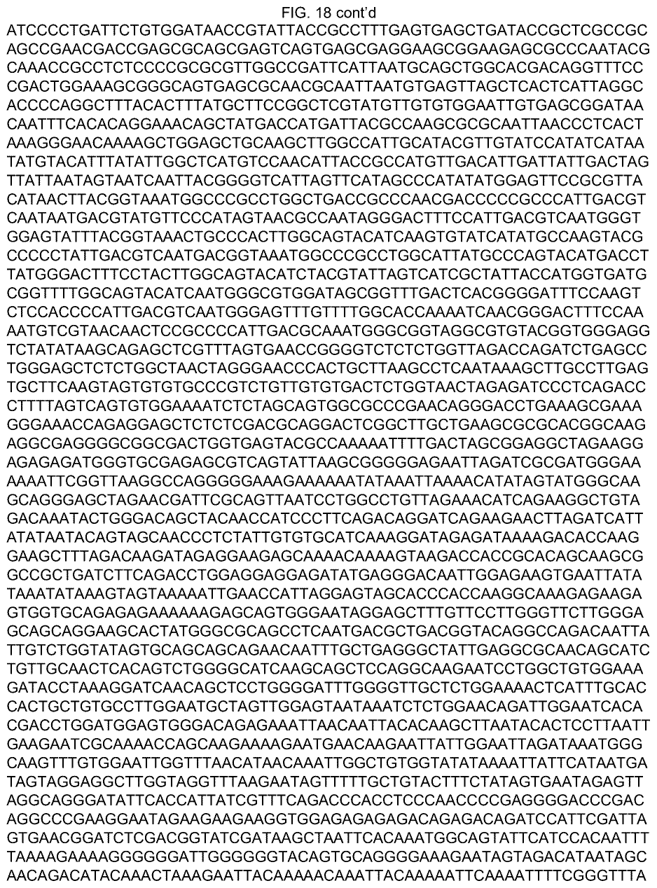

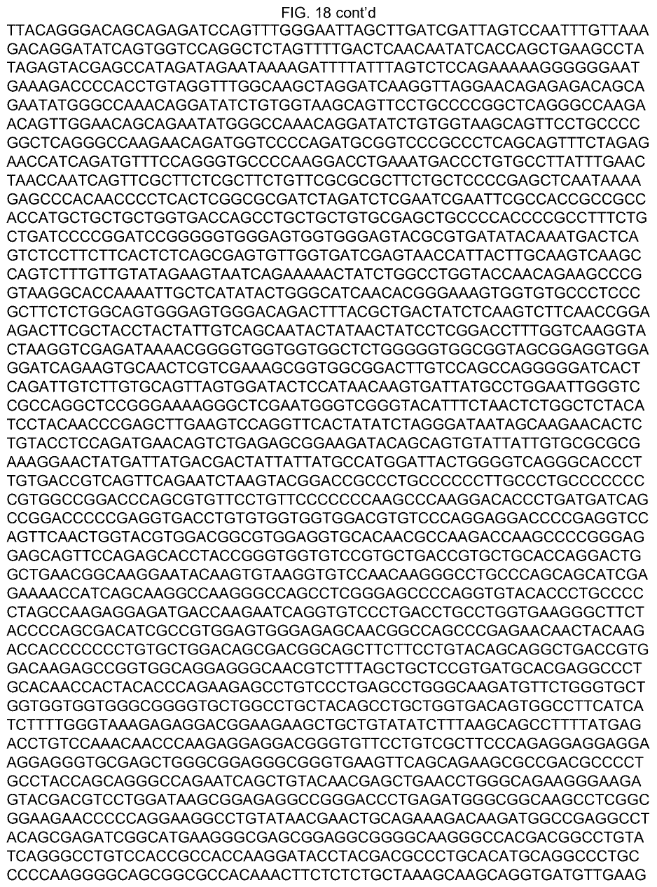

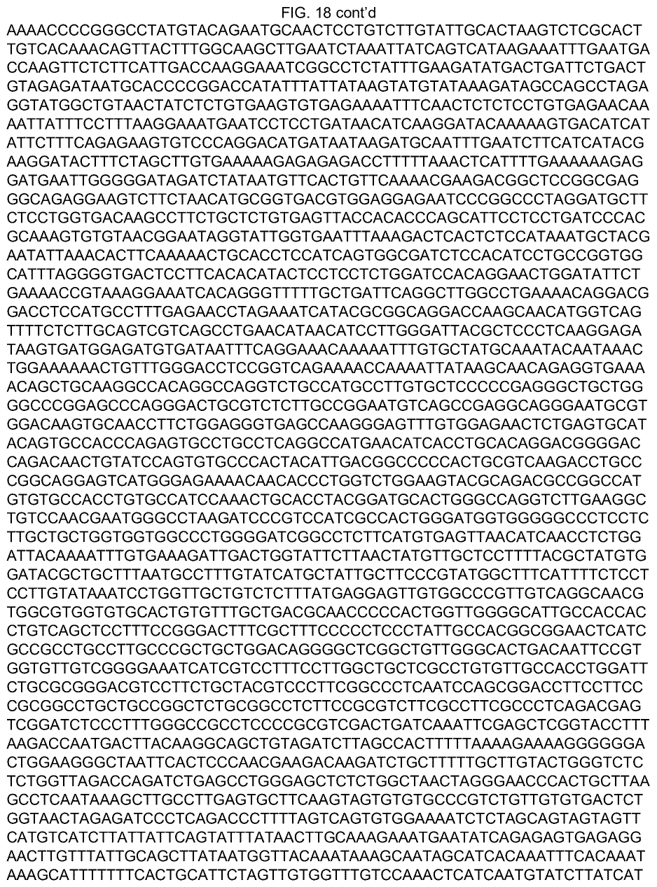

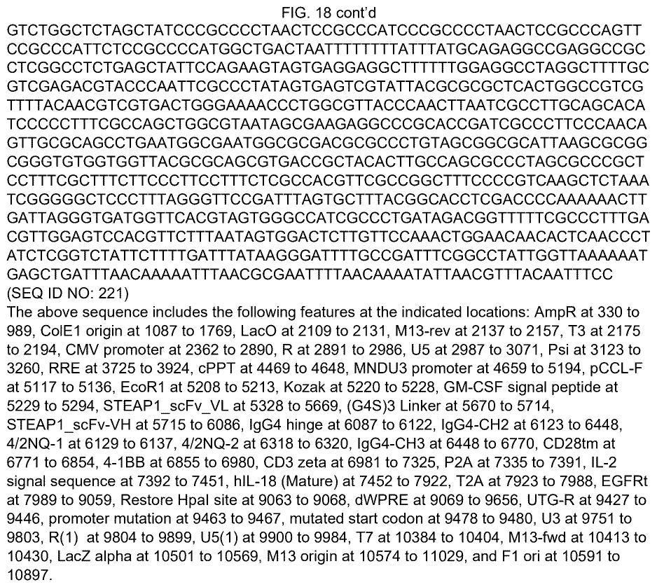

(X) Kits. The current disclosure also includes kits. Kits can include various components to practice methods disclosed herein. For example, depending on the aspect of the methods practiced, kits could include one or more of nucleic acids encoding a genetic construct disclosed herein; a protein or encoding sequence as set forth in FIG. 18; nucleic acids encoding a pro-inflammatory cytokine; nucleic acids encoding IL-18; nucleic acids encoding a self-cleaving peptide; nucleic acids encoding recombinant receptors; nucleic acids encoding chimeric antigen receptors (CAR); lentiviral genetic construct; a nucleic acid encoding an scFv; a nucleic acid encoding a VL; a nucleic acid encoding a VH; a nucleic acid encoding a transmembrane domain; a nucleic acid encoding EGFRt; a nucleic acid encoding CD19t; cells (e.g., immune cells, T-cells, CD4 T cells, CD8 T cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC), untransduced T cells, STEAP1-BBζ CAR T cells, pro-inflammatory cytokine-expressing immune cells, pro-inflammatory cytokine-expressing CAR T cells, 22Rv1 STEAP1 knockout (ko) cells, 22Rv1 STEAP1 ko cells with rescue of STEAP1 expression by lentiviral expression); cell lines (e.g., androgen receptor (AR)-positive human prostate cancer cell lines, AR-negative prostate cancer cell lines, DU145 cell lines, DU145 STEAP1 cell lines, lentivirally engineered DU145 hSTEAP1 cell lines, lentivirally engineered DU145 mSteap1 cell lines, C4-2B prostate cancer cell lines, PC3 prostate cancer cell lines, LNCaP cell lines, 22Rv1 cell lines, VCaP cell lines, LNCaP95 cell lines, MSKCCEF1 cell lines, NCI-H660 cell lines); tissue samples (e.g., peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom); genetic expression components (e.g., genes for expression provided by vectors (e.g., lentiviral vector, retroviral vector), CRISPR components, ZFNs, TALENs, MegaTALs, targeted viral vectors and/or nanoparticles); cell formulation or activation components (e.g., saline, buffered saline, phosphate buffered saline (PBS); biocompatible buffers such as, Ca++/Mg++ free PBS; physiological saline, water, Hanks' solution, Ringer's solution, T cell stimulating epitopes (e.g., anti-CD3/anti-CD28 conjugated beads; OKT3, TGN1412), culture-initiating compositions, RPMI, non-essential amino acids, sodium pyruvate, penicillin/streptomycin, non-dividing EBV-transformed lymphoblastoid cells (LCL), IL-21, human serum albumin (HSA) or other human serum components or fetal bovine serum, dextrose, Stabilizers, preservatives); combination therapy components (e.g., local anesthetics, chemotherapeutic agents, immunosuppressive agents, anti-inflammatory agents); an antibody tagged with a fluorescent molecule; PCR amplification sequences; cytokines (e.g., IL-2, IL-7, IL-15, IL-21); culture vessels; reference levels, hSTEAP1-KI mice bearing syngeneic, disseminated prostate cancer; primer pairs to amplify portions of wildtype or hSTEAP1-KI alleles; GAPDH; IFN-γ enzyme-linked immunosorbent assay (ELISA); culture plates; etc.

The Exemplary Embodiments and Examples below are included to demonstrate particular, non-limiting embodiments of the disclosure. Those of ordinary skill in the art will recognize in light of the present disclosure that many changes can be made to the specific embodiments disclosed herein and still obtain a like or similar result without departing from the spirit and scope of the disclosure.

### (XI) Exemplary Embodiments

1. A genetic construct including (i) a sequence encoding interleukin-18 (IL-18), and (ii) a sequence encoding a chimeric antigen receptor (CAR) including a STEAP1 binding domain, a spacer, a transmembrane domain, and an intracellular component.

2. A genetic construct including (i) a sequence encoding a pro-inflammatory cytokine, and (ii) a sequence encoding a recombinant receptor including an extracellular component, an intracellular component, and a transmembrane domain linking the extracellular component to the intracellular component, wherein the extracellular component includes a STEAP1 binding domain.

3. The genetic construct of embodiment 2, wherein the pro-inflammatory cytokine includes interleukin (IL)-18, IL1α, IL-1β, IL-33, IL-36α, IL-36β, or IL-36γ.

4. The genetic construct of embodiments 2 or 3, wherein the pro-inflammatory cytokine includes a sequence as set forth in any of SEQ ID NOs: 4, 6, 7, 8, 9, 10, 11, 12, or 13, or a sequence having at least 95% sequence identity to the sequence as set forth in any of SEQ ID NOs: 4, 6, 7, 8, 9, 10, 11, 12, or 13.

5. The genetic construct of any of embodiments 2-4, wherein the pro-inflammatory cytokine includes IL-18.

6. The genetic construct of embodiment 5, wherein the IL-18 includes the sequence as set forth in SEQ ID NO: 4 or SEQ ID NO: 6 or a sequence having at least 95% sequence identity to the sequence as set forth in SEQ ID NO: 4 or SEQ ID NO: 6.

7. The genetic construct of embodiments 5 or 6, wherein the IL-18 is encoded by the sequence as set forth in SEQ ID NO: 5 or a sequence having at least 95% sequence identity to the sequence as set forth in SEQ ID NO: 5.

8. The genetic construct of any of embodiments 2-7, wherein the recombinant receptor includes a chimeric antigen receptor (CAR), an engineered T cell receptor (eTCR), or a hybrid thereof.

9. The genetic construct of any of embodiments 2-8, wherein the recombinant receptor includes a CAR.

10. The genetic construct of any of embodiments 2-9, wherein the STEAP1 binding domain includes
a variable heavy chain including a complementarity determining region (CDRH) 1 as set forth in SEQ ID NO: 14, a CDRH2 as set forth in SEQ ID NO: 15, and a CDRH3 as set forth in SEQ ID NO: 16, and a variable light chain including a CDRL1 as set forth in SEQ ID NO: 17, a CDRL2 as set forth in SEQ ID NO: 18, and a CDRL3 as set forth in SEQ ID NO: 19, according to Kabat;
a variable heavy chain including a CDRH1 as set forth in SEQ ID NO: 20, a CDRH2 as set forth in SEQ ID NO: 21, and a CDRH3 as set forth in SEQ ID NO: 16, and a variable light chain including a CDRL1 as set forth in SEQ ID NO: 17, a CDRL2 as set forth in SEQ ID NO: 18, and a CDRL3 as set forth in SEQ ID NO: 19, according to Chothia;
a variable heavy chain including a CDRH1 as set forth in SEQ ID NO: 22, a CDRH2 as set forth in SEQ ID NO: 23, and a CDRH3 as set forth in SEQ ID NO: 24, and a variable light chain including a CDRL1 as set forth in SEQ ID NO: 25, a CDRL2 having the sequence WAS, and a CDRL3 as set forth in SEQ ID NO: 19, according to IMGT;
a variable heavy chain including a CDRH1 as set forth in SEQ ID NO: 26, a CDRH2 as set forth in SEQ ID NO: 27, and a CDRH3 as set forth in SEQ ID NO: 24, and a variable light chain including a CDRL1 as set forth in SEQ ID NO: 17, a CDRL2 as set forth in SEQ ID NO: 28, and a CDRL3 as set forth in SEQ ID NO: 19, according to North; or
a variable heavy chain including a CDRH1 as set forth in SEQ ID NO: 29, a CDRH2 as set forth in SEQ ID NO: 30, and a CDRH3 as set forth in SEQ ID NO: 31, and a variable light chain including a CDRL1 as set forth in SEQ ID NO: 32, a CDRL2 as set forth in SEQ ID NO: 33, and a CDRL3 as set forth in SEQ ID NO: 34, according to Contact.

11. The genetic construct of any of embodiments 2-10, wherein the STEAP1 binding domain includes a variable heavy chain with at least 90% sequence identity to the sequence as set forth in SEQ ID NO: 35 and a variable light chain with at least 90% sequence identity to the sequence as set forth in SEQ ID NO: 36.

12. The genetic construct of any of embodiments 2-11, wherein the STEAP1 binding domain includes a variable heavy chain that has the sequence as set forth in SEQ ID NO: 35 and a variable light chain that has the sequence as set forth in SEQ ID NO: 36.

13. The genetic construct of any of embodiments 2-12, wherein the STEAP1 binding domain includes a variable heavy chain that is encoded by a sequence having at least 90% sequence identity to the sequence as set forth in SEQ ID NO: 72 and a variable light chain that is encoded by a sequence having at least 90% sequence identity to the sequence as set forth in SEQ ID NO: 70.

14. The genetic construct of any of embodiments 2-13, wherein the STEAP1 binding domain includes a variable heavy chain that is encoded by the sequence as set forth in SEQ ID NO: 72 and a variable light chain that is encoded by the sequence as set forth in SEQ ID NO: 70.

15. The genetic construct of any of embodiments 2-14, wherein the STEAP1 binding domain includes a single chain variable fragment (scFv).

16. The genetic construct of embodiment 15, wherein the scFv has at least 90% sequence identity to the sequence as set forth in SEQ ID NO: 73 or SEQ ID NO: 75.

17. The genetic construct of embodiments 15 or 16, wherein the scFv has the sequence as set forth in SEQ ID NO: 73 or SEQ ID NO: 75.

18. The genetic construct of any of embodiments 15-17, wherein the scFv is encoded by a sequence having at least 90% sequence identity to the sequence as set forth in SEQ ID NO: 74 or SEQ ID NO: 76.

19. The genetic construct of any of embodiments 15-18, wherein the scFv is encoded by the sequence as set forth in SEQ ID NO: 74 or SEQ ID NO: 76.

20. The genetic construct of any of embodiments 2-19, wherein the extracellular component further includes a spacer.

21. The genetic construct of embodiment 20, wherein the spacer includes a long spacer, an intermediate spacer, or a short spacer.

22. The genetic construct of embodiment 21, wherein the long spacer is 230 amino acids or less.

23. The genetic construct of embodiments 21 or 22, wherein the long spacer includes a IgG4 hinge region, IgG4-CH2 domain, and IgG4-CH3 domain.

24. The genetic construct of embodiment 23, wherein the IgG4 is human IgG4.

25. The genetic construct of any of embodiments 21-24, wherein the long spacer includes a sequence having at least 95% sequence identity to the sequence as set forth in SEQ ID NO: 87.

26. The genetic construct of any of embodiments 21-25, wherein the long spacer includes the sequence as set forth in SEQ ID NO: 87.

27. The genetic construct of any of embodiments 21-26, wherein the long spacer is encoded by a sequence having at least 95% sequence identity to the sequence as set forth in SEQ ID NO: 88.

28. The genetic construct of any of embodiments 21-27, wherein the long spacer is encoded by the sequence as set forth in SEQ ID NO: 88.

29. The genetic construct of embodiment 21, wherein the intermediate spacer includes a hinge region and an IgG4-CH3 domain.

30. The genetic construct of embodiments 21 or 29, wherein the intermediate spacer includes the sequence as set forth in SEQ ID NO: 222 or a sequence having at least 95% sequence identity to the sequence as set forth in SEQ ID NO: 222.

31. The genetic construct of any of embodiments 21, 29 or 30, wherein the intermediate spacer is encoded by the sequence as set forth in SEQ ID NO: 86 or a sequence having at least 95% sequence identity to the sequence as set forth in SEQ ID NO: 86.

32. The genetic construct of embodiment 21, wherein the short spacer includes an IgG4 hinge region.

33. The genetic construct of embodiments 21 or 32, wherein the short spacer includes the sequence as set forth in SEQ ID NO: 77 or a sequence having at least 95% sequence identity to the sequence as set forth in SEQ ID NO: 77.

34. The genetic construct of any of embodiments 21, 32 or 33, wherein the short spacer is encoded by the sequence as set forth in any of SEQ ID NOs: 78-80 or a sequence having at least 95% sequence identity to the sequence as set forth in any of SEQ ID NOs: 78-80.

35. The genetic construct of any of embodiments 2-34, wherein the intracellular component includes all or a portion of a CD3ζ signaling domain; all or a portion of a 4-1BB signaling domain, all or a portion of a CD28 signaling domain, all or a portion of the CD3ζ signaling domain and the 4-1BB signaling domain; all or a portion of the CD3ζ signaling domain and all or a portion of the CD28 signaling domain; or all or a portion of the CD3ζ signaling domain, all or a portion of the 4-1BB signaling domain, and all or a portion of the CD28 signaling domain.

36. The genetic construct of any of embodiments 2-35, wherein the intracellular component includes all or a portion of a CD3ζ signaling domain and all or a portion of a 4-1BB signaling domain.

37. The genetic construct of embodiment 36, wherein the CD3ζ signaling domain has at least 90% sequence identity to the sequence as set forth in any of SEQ ID NOs: 89, 90, or 223.

38. The genetic construct of embodiments 36 or 37, wherein the CD3ζ signaling domain has the sequence as set forth in any of SEQ ID NOs: 89, 90, or 223.

39. The genetic construct of any of embodiments 36-38, wherein the CD3ζ signaling domain is encoded by a sequence having at least 90% sequence identity to the sequence as set forth in SEQ ID NOs: 91 or 92.

40. The genetic construct of any of embodiments 36-39, wherein the CD3ζ signaling domain is encoded by the sequence as set forth in SEQ ID NOs: 91 or 92.

41. The genetic construct of any of embodiments 36-40, wherein the 4-1BB signaling domain has at least 90% sequence identity to the sequence as set forth in any of SEQ ID NOs: 93, 94, or 95.

42. The genetic construct of any of embodiments 36-41, wherein the 4-1BB signaling domain has the sequence as set forth in any of SEQ ID Nos: 93, 94, or 95.

43. The genetic construct of any of embodiments 36-42, wherein the 4-1BB signaling domain is encoded by a sequence having at least 90% sequence identity to the sequence as set forth in any of SEQ ID NOs: 96, 97, or 98.

44. The genetic construct of any of embodiments 36-43, wherein the 4-1BB signaling domain is encoded by the sequence as set forth in any of SEQ ID NOs: 96, 97, or 98.

45. The genetic construct of any of embodiments 2-44, wherein the transmembrane domain includes a CD28 transmembrane domain.

46. The genetic construct of embodiment 45, wherein the CD28 transmembrane domain has at least 90% sequence identity to the sequence as set forth in any of SEQ ID NOs: 99, 100, or 101.

47. The genetic construct of embodiments 45 or 46, wherein the CD28 transmembrane domain has the sequence as set forth in any of SEQ ID NOs: 99, 100, or 101.

48. The genetic construct of any of embodiments 45-47, wherein the CD28 transmembrane domain is encoded by a sequence having at least 90% sequence identity to the sequence as set forth in any of SEQ ID NOs: 102, 103, 104, or 105.

49. The genetic construct of any of embodiments 45-48, wherein the CD28 transmembrane domain is encoded by the sequence as set forth in any of SEQ ID NOs: 102, 103, 104, or 105.

50. The genetic construct of any of embodiments 2-49, wherein the STEAP1 binding domain includes a STEAP1 scFv, the intracellular component includes a CD3ζ signaling domain and a 4-1BB signaling domain, and the transmembrane domain includes a CD28 transmembrane domain.

51. The genetic construct of any of embodiments 2-50, wherein the recombinant receptor includes a sequence having at least 90% sequence identity to the sequence as set forth in SEQ ID NO: 214.

52. The genetic construct of any of embodiments 2-51, wherein the recombinant receptor includes the sequence as set forth in SEQ ID NO: 214.

53. The genetic construct of any of embodiments 2-52, wherein the recombinant receptor is encoded by a sequence having at least 90% sequence identity to the sequence as set forth in SEQ ID NO: 215.

54. The genetic construct of any of embodiments 2-53, wherein the recombinant receptor is encoded by the sequence as set forth in SEQ ID NO: 215.

55. The genetic construct of any of embodiments 2-54, further including a tag cassette or a suicide switch.

56. The genetic construct of any of embodiments 2-55, wherein the recombinant receptor further includes a multimerization domain.

57. The genetic construct of any of embodiments 2-56, further including a self-cleaving polypeptide.

58. The genetic construct of embodiment 57, wherein the self-cleaving polypeptide includes a porcine teschovirus-1 (P2A), Thosea asigna virus (T2A), equine rhinitis A virus (E2A), foot-and-mouth disease virus (F2A), or variants thereof.

59. The genetic construct of embodiments 57 or 58, further including a second self-cleaving polypeptide.

60. The genetic construct of embodiment 59, wherein the second self-cleaving polypeptide includes a porcine teschovirus-1 (P2A), Thosea asigna virus (T2A), equine rhinitis A virus (E2A), foot-and-mouth disease virus (F2A), or variants thereof.

61. The genetic construct of any of embodiments 2-60, further including a transduction marker.

62. The genetic construct of embodiment 61, wherein the transduction marker is a truncated epidermal growth factor receptor (EGFRt).

63. The genetic construct of embodiment 62, wherein the EGFRt includes a sequence having at least 90% sequence identity to the sequence as set forth in SEQ ID NO: 106.

64. The genetic construct of embodiments 62 or 63, wherein the EGFRt includes the sequence as set forth in SEQ ID NO: 106.

65. The genetic construct of any of embodiments 62-64, wherein the EGFRt is encoded by a sequence having at least 90% sequence identity to the sequence as set forth in SEQ ID NO: 107.

66. The genetic construct of any of embodiments 62-65, wherein the EGFRt is encoded by the sequence as set forth in SEQ ID NO: 107.

67. The genetic construct of any of embodiments 2-66, further including a selection cassette.

68. The genetic construct of embodiment 67, wherein the selection cassette includes dihydrofolate reductase double mutant (DHFRdm).

69. The genetic construct of any of embodiments 2-68, wherein the genetic construct is encoded by a sequence having at least 90% sequence identity to the sequence as set forth in any of SEQ ID NO: 217, 219, 220, or 221.

70. The genetic construct of any of embodiments 2-69, wherein the genetic construct is encoded by the sequence as set forth in any of SEQ ID NOs: 217, 219, 220, or 221.

71. A nanoparticle encapsulating the genetic construct of any of embodiments 1-70.

72. A cell genetically modified to express the genetic construct of any of embodiments 1-70.

73. The cell of embodiment 72, wherein the cell is an autologous cell or an allogeneic cell in reference to a subject.

74. The cell of embodiments 72 or 73, wherein the cell is in vivo or ex vivo.

75. The cell of any of embodiments 72-74, wherein the cell is an immune cell.

76. The cell of any of embodiments 72-75, wherein the cell is a T cell, B cell, natural killer (NK) cell, NK-T cell, monocyte/macrophage, hematopoietic stem cells (HSC), or a hematopoietic progenitor cell (HPC).

77. The cell of embodiment 76, wherein the cell is a T cell selected from a CD3+ T cell, a CD4+ T cell, a CD8+ T cell, a central memory T cell, an effector memory T cell, and/or a naïve T cell.

78. The cell of any of embodiments 72-77, wherein the cell is a CD8+ T cell.

79. The cell of any of embodiments 72-77, wherein the cell is a CD4+ T cell.

80. The cell of any of embodiments 72-79, wherein the cell is non-reactive against cells expressing Steap1b.

81. A population of cells genetically modified to express the genetic construct of any of embodiments 1-70.

82. The population of cells of embodiment 81, wherein the population of cells include autologous cells or allogeneic cells in reference to a subject.

83. The population of cells of embodiments 81 or 82, wherein the population of cells are in vivo or ex vivo.

84. The population of cells of any of embodiments 81-83, wherein the population of cells include immune cells.

85. The population of cells of any of embodiments 81-84, wherein the population of cells include T cells, B cells, natural killer (NK) cells, NK-T cells, monocytes/macrophages, hematopoietic stem cells (HSC), and/or hematopoietic progenitor cell (HPCs).

86. The population of cells of any of embodiments 81-85, wherein the population of cells include CD4+ T cells and CD8+ T cells.

87. The population of cells of any of embodiments 81-86, wherein the population of cells include a 1:1 ratio of CD4+ T cells to CD8+ T cells.

88. The population of cells of any of embodiments 81-87, wherein the population of cells are non-reactive against cells expressing Steap1b.

89. A formulation including (i) cells genetically modified to express the genetic construct of any of embodiments 1-70 and (ii) a pharmaceutically acceptable carrier.

90. A method of treating a subject with a STEAP1-related disorder including co-expressing a therapeutically effective amount of a STEAP1 recombinant receptor and a pro-inflammatory cytokine within the subject, thereby treating the subject.

91. The method of embodiment 90, wherein the pro-inflammatory cytokine includes IL-18, IL1α, IL-1β, IL-33, IL-36α, IL-36β, or IL-36γ.

92. The method of embodiments 90 or 91, wherein the pro-inflammatory cytokine includes IL-18.

93. The method of any of embodiments 90-92, wherein the STEAP1 recombinant receptor include a STEAP1 CAR.

94. The method of embodiment 93, wherein the STEAP1 CAR includes an extracellular component including a STEAP1 binding domain; an intracellular component including a CD3ζ signaling domain and a 4-1BB signaling domain; and a transmembrane domain linking the extracellular component to the intracellular component, wherein the transmembrane domain includes a CD28 transmembrane domain.

95. The method of any of embodiments 90-94, wherein the co-expressing includes administering a therapeutically effective amount of the formulation of embodiment 89 to the subject.

96. The method of any of embodiments 90-95, wherein the subject's STEAP1-related disorder is based on presence of diseased cells expressing STEAP1 at low STEAP1 antigen conditions.

97. The method of embodiment 96, wherein the low STEAP1 antigen conditions include less than 50,000 STEAP1 molecules per diseased cell.

98. The method of embodiments 96 or 97, wherein the low STEAP1 antigen conditions include less than 30,000 STEAP1 molecules per diseased cell.

99. The method of any of embodiments 96-98, wherein the low STEAP1 antigen conditions include less than 15,000 STEAP1 molecules per diseased cell.

100. The method of any of embodiments 96-99, wherein the low STEAP1 antigen conditions include less than 10,000 STEAP1 molecules per diseased cell.

101. The method of any of embodiments 96-100, wherein the low STEAP1 antigen conditions include less than 5,000 STEAP1 molecules per diseased cell.

102. The method of any of embodiments 96-101, wherein low STEAP1 antigen conditions include less than 2,000 STEAP1 molecules per diseased cell.

103. The method of any of embodiments 96-102, wherein low STEAP1 antigen conditions include less than 1,500 STEAP1 molecules per diseased cell.

104. The method of any of embodiments 96-103, further including obtaining a sample of the diseased cells and measuring the STEAP1 antigen density levels of the cells.

105. The method of any of embodiments 90-104, wherein the STEAP1-related disorder includes prostate cancer, the Ewing family of tumors (EFT), bladder cancer, ovarian cancer, or rhabdomyosarcoma.

106. The method of embodiment 105, wherein the EFT includes Ewing Sarcoma.

107. The method of embodiment 106 wherein the Ewing Sarcoma includes orthotopic Ewing sarcoma or disseminated Ewing sarcoma.

108. The method of any of embodiments 90-107, wherein the STEAP1-related disorder includes lethal, metastatic castration-resistant prostate cancer.

109. The method of any of embodiments 95-108, wherein the administering a therapeutically effective amount includes administering intravesically, intravenously, intradermally, intraarterially, intraparenterally, intranodally, intralymphatically, intraperitoneally, intralesionally, intraprostatically, intravaginally, intrarectally, topically, intrathecally, intratumorally, intramuscularly, or subcutaneously.

110. A method of providing an immune response against STEAP1-expressing cells in a subject in need thereof including co-expressing a therapeutically effective amount of a STEAP1 recombinant receptor and a pro-inflammatory cytokine within the subject, thereby providing an immune response against STEAP1-expressing cells in the subject.

111. The method of embodiment 110, wherein the pro-inflammatory cytokine includes IL-18, IL1α, IL-1β, IL-33, IL-36α, IL-36β, or IL-36γ.

112. The method of embodiments 110 or 111, wherein the pro-inflammatory cytokine includes IL-18.

113. The method of any of embodiments 110-112, wherein the STEAP1 recombinant receptor include a STEAP1 CAR.

114. The method of embodiment 113, wherein the STEAP1 CAR includes an extracellular component including a STEAP1 binding domain; an intracellular component including a CD3ζ signaling domain and a 4-1BB signaling domain; and a transmembrane domain linking the extracellular component to the intracellular component, wherein the transmembrane domain includes a CD28 transmembrane domain.

115. The method of any of embodiments 110-114, wherein the co-expressing includes administering a therapeutically effective amount of the formulation of any of embodiments 89 to the subject.

116. The method of any of embodiments 110-115, wherein the STEAP1-expressing cells include prostate cancer cells, the Ewing family of tumor (EFT) cells, bladder cancer cells, ovarian cancer cells, or rhabdomyosarcoma cells.

117. The method of embodiment 116, wherein the EFT includes Ewing Sarcoma.

118. The method of embodiment 117, wherein the Ewing Sarcoma includes orthotopic Ewing sarcoma or disseminated Ewing sarcoma.

119. The method of any of embodiments 110-118, wherein the STEAP1-expressing cells include prostate cancer cells.

120. The method of embodiment 119, wherein the prostate cancer cells include lethal, metastatic castration-resistant prostate cancer cells.

121. The method of any of embodiments 115-120, wherein the administering a therapeutically effective amount includes administering intravesically, intravenously, intradermally, intraarterially, intraparenterally, intranodally, intralymphatically, intraperitoneally, intralesionally, intraprostatically, intravaginally, intrarectally, topically, intrathecally, intratumorally, intramuscularly, or subcutaneously.

122. A kit including nucleic acids encoding a pro-inflammatory cytokine and nucleic acids encoding a recombinant receptor including an extracellular component, an intracellular component, and a transmembrane domain linking the extracellular component to the intracellular component, wherein the extracellular component includes a STEAP1 binding domain.

123. The kit of embodiment 122, wherein the pro-inflammatory cytokine includes interleukin (IL)-18, IL1α, IL-1β, IL-33, IL-36α, IL-36β, or IL-36γ.

124. The kit of embodiments 122 or 123, wherein the pro-inflammatory cytokine includes the sequence as set forth in any of SEQ ID NOs: 4, 6, 7, 8, 9, 10, 11, 12, or 13 or a sequence having at least 95% sequence identity to the sequence as set forth in any of SEQ ID NOs: 4, 6, 7, 8, 9, 10, 11, 12, or 13.

125. The kit of any of embodiments 122-124, wherein the pro-inflammatory cytokine includes IL-18.

126. The kit of any of embodiments 122-125, wherein the IL-18 includes the sequence as set forth in SEQ ID NO: 4 or SEQ ID NO: 6 or a sequence having at least 95% sequence identity to the sequence as set forth in SEQ ID NO: 4 or SEQ ID NO: 6.

127. The kit of embodiments 125 or 126, wherein the IL-18 is encoded by the sequence as set forth in SEQ ID NO: 5 or a sequence having at least 95% sequence identity to the sequence as set forth in SEQ ID NO: 5.

128. The kit of any of embodiments 122-127, wherein the recombinant receptor includes a chimeric antigen receptor (CAR), an engineered T cell receptor (eTCR), or a hybrid thereof.

129. The kit of any of embodiments 122-128, wherein the recombinant receptor includes a CAR.

130. The kit of any of embodiments 122-129, wherein the STEAP1 binding domain includes
a variable heavy chain including a complementarity determining region (CDRH) 1 as set forth in SEQ ID NO: 14, a CDRH2 as set forth in SEQ ID NO: 15, and a CDRH3 as set forth in SEQ ID NO: 16, and a variable light chain including a CDRL1 as set forth in SEQ ID NO: 17, a CDRL2 as set forth in SEQ ID NO: 18, and a CDRL3 as set forth in SEQ ID NO: 19, according to Kabat;
a variable heavy chain including a CDRH1 as set forth in SEQ ID NO: 20, a CDRH2 as set forth in SEQ ID NO: 21, and a CDRH3 as set forth in SEQ ID NO: 16, and a variable light chain including a CDRL1 as set forth in SEQ ID NO: 17, a CDRL2 as set forth in SEQ ID NO: 18, and a CDRL3 as set forth in SEQ ID NO: 19, according to Chothia;
a variable heavy chain including a CDRH1 as set forth in SEQ ID NO: 22, a CDRH2 as set forth in SEQ ID NO: 23, and a CDRH3 as set forth in SEQ ID NO: 24, and a variable light chain including a CDRL1 as set forth in SEQ ID NO: 25, a CDRL2 having the sequence WAS, and a CDRL3 as set forth in SEQ ID NO: 19, according to IMGT;
a variable heavy chain including a CDRH1 as set forth in SEQ ID NO: 26, a CDRH2 as set forth in SEQ ID NO: 27, and a CDRH3 as set forth in SEQ ID NO: 24, and a variable light chain including a CDRL1 as set forth in SEQ ID NO: 17, a CDRL2 as set forth in SEQ ID NO: 28, and a CDRL3 as set forth in SEQ ID NO: 19, according to North; or
a variable heavy chain including a CDRH1 as set forth in SEQ ID NO: 29, a CDRH2 as set forth in SEQ ID NO: 30, and a CDRH3 as set forth in SEQ ID NO: 31, and a variable light chain including a CDRL1 as set forth in SEQ ID NO: 32, a CDRL2 as set forth in SEQ ID NO: 33, and a CDRL3 as set forth in SEQ ID NO: 34, according to Contact.

131. The kit of any of embodiments 122-130, wherein the STEAP1 binding domain includes a variable heavy chain with at least 90% sequence identity to the sequence as set forth in SEQ ID NO: 35 and a variable light chain with at least 90% sequence identity to the sequence as set forth in SEQ ID NO: 36.

132. The kit of any of embodiments 122-131, wherein the STEAP1 binding domain includes a variable heavy chain that has the sequence as set forth in SEQ ID NO: 35 and a variable light chain that has the sequence as set forth in SEQ ID NO: 36.

133. The kit of any of embodiments 122-132, wherein the STEAP1 binding domain includes a variable heavy chain that is encoded by a sequence having at least 90% sequence identity to the sequence as set forth in SEQ ID NO: 72 and a variable light chain that is encoded by a sequence having at least 90% sequence identity to the sequence as set forth in SEQ ID NO: 70.

134. The kit of any of embodiments 122-133, wherein the STEAP1 binding domain includes a variable heavy chain that is encoded by the sequence as set forth in SEQ ID NO: 72 and a variable light chain that is encoded by the sequence as set forth in SEQ ID NO: 70.

135. The kit of any of embodiments 122-134, wherein the STEAP1 binding domain includes a single chain variable fragment (scFv).

136. The kit of embodiment 135, wherein the scFv has at least 90% sequence identity to the sequence as set forth in SEQ ID NO: 73 or SEQ ID NO: 75.

137. The kit of embodiments 135 or 136, wherein the scFv has the sequence as set forth in SEQ ID NO: 73 or SEQ ID NO: 75.

138. The kit of any of embodiments 135-137, wherein the scFv is encoded by a sequence having at least 90% sequence identity to the sequence as set forth in SEQ ID NO: 74 or SEQ ID NO: 76.

139. The kit of any of embodiments 135-138, wherein the scFv is encoded by the sequence as set forth in SEQ ID NO: 74 or SEQ ID NO: 76.

140. The kit of any of embodiments 122-139, wherein the extracellular component further includes a spacer.

141. The kit of embodiment 140, wherein the spacer includes a long spacer, an intermediate spacer, or a short spacer.

142. The kit of any of embodiments 122-141, wherein the intracellular component includes all or a portion of a CD3ζ signaling domain; all or a portion of a 4-1BB signaling domain, all or a portion of a CD28 signaling domain, all or a portion of the CD3ζ signaling domain and the 4-1BB signaling domain; all or a portion of the CD3ζ signaling domain and all or a portion of the CD28 signaling domain; or all or a portion of the CD3ζ signaling domain, all or a portion of the 4-1BB signaling domain, and all or a portion of the CD28 signaling domain.

143. The kit of any of embodiments 122-142, wherein the STEAP1 binding domain includes a STEAP1 scFv, the intracellular component includes a CD3ζ signaling domain and a 4-1BB signaling domain, and the transmembrane domain includes a CD28 transmembrane domain.

144. The kit of any of embodiments 122-143, wherein the recombinant receptor includes a sequence having at least 90% sequence identity to the sequence as set forth in SEQ ID NO: 214.

145. The kit of any of embodiments 122-144, wherein the recombinant receptor includes the sequence as set forth in SEQ ID NO: 214.

146. The kit of any of embodiments 122-145, wherein the nucleic acids encoding the recombinant receptor include a sequence having at least 90% sequence identity to the sequence as set forth in SEQ ID NO: 215.

147. The kit of any of embodiments 122-146, wherein the nucleic acids encoding the recombinant receptor include the sequence as set forth in SEQ ID NO: 215.

148. The kit of any of embodiments 122-147, wherein the nucleic acids encoding the pro-inflammatory cytokine and/or nucleic acids encoding the recombinant receptor are encapsulated within a liposome or polymeric nanoparticle.

149. The kit of any of embodiments 122-148, wherein the nucleic acid encoding the pro-inflammatory cytokine and/or nucleic acids encoding the recombinant receptor are within a viral vector.

150. The kit of embodiment 149, wherein the viral vector is a retroviral vector.

151. The kit of embodiment 150, wherein the retroviral vector is a lentiviral vector.

152. The kit of any of embodiments 122-151, further including guide RNA.

### (XII) Experimental Example. Targeting STEAP1 with Chimeric Antigen Receptor T Cell Therapy in Advanced Prostate Cancer

Methods. Cell lines. 22Rv1 (CRL-2505), LNCaP (CRL-1740), PC3 (CRL-1435), DU145 (HTB-81), NCI-H660 (CRL-5813), C4-2B (CRL-3315), RM9 (RL-3312), and Myc-CaP (CRL-3255) were obtained from the American Type Culture Collection. LNCaP95 cells were a gift from University of Washington. MSKCC EF1 were derived from the MSKCC PCa4 organoid line provided by Memorial Sloan Kettering Cancer Center, as previously described (Lee, J. K., et al., PNAS 115, E4473-e4482 (2018)). Cell lines were maintained in RPMI 1640 medium supplemented with 10% FBS, 100 U/mL penicillin and 100 μg/mL streptomycin, and 4 mmol/L GlutaMAX (Thermo Fisher). 22Rv1 STEAP1 ko and PC3 STEAP1 ko cells were generated by transient transfection of 22Rv1 cells with a pool of PX458 (Addgene #48138) plasmids each expressing one of four different sgRNA targeting sequences predicted from the Broad Institute Genetic Perturbation Platform sgRNA Designer (Doench, J. G., et al., Nat. Biotechnol. 34, 184-191 (2016)): 1) 5′-ATAGTCTGTCTTACCCAATG-3′ (SEQ ID NO: 64); 2) 5′-CCTTTGTAGCATAAGGACAC-3′ (SEQ ID NO: 65); 3) 5′-ATCCACTTATCCAACCAATG-3′ (SEQ ID NO: 66); and 4) 5′-CATCAACAAAGTCTTGCCAA-3′ (SEQ ID NO: 67). 48-72 hours after transfection, GFP-positive cells were singly sorted on a Sony SH800 Cell Sorter into a 96-well plate and clonally expanded. Human STEAP, mouse Steap1, mouse Steap1 reconstituted with human STEAP1 ectodomains, and human STEAP1B cDNAs were cloned into the EcoRI site of the third-generation lentiviral vector FU-CGW (Xin, L., et al., PNAS 103, 7789-7794 (2006)) by Gibson assembly. Lentiviruses were generated and titered as previously described (Xin, L., et al., PNAS 100, 11896-11903 (2003)) and used to transduce 22Rv1 STEAP1 ko or DU145 cells.

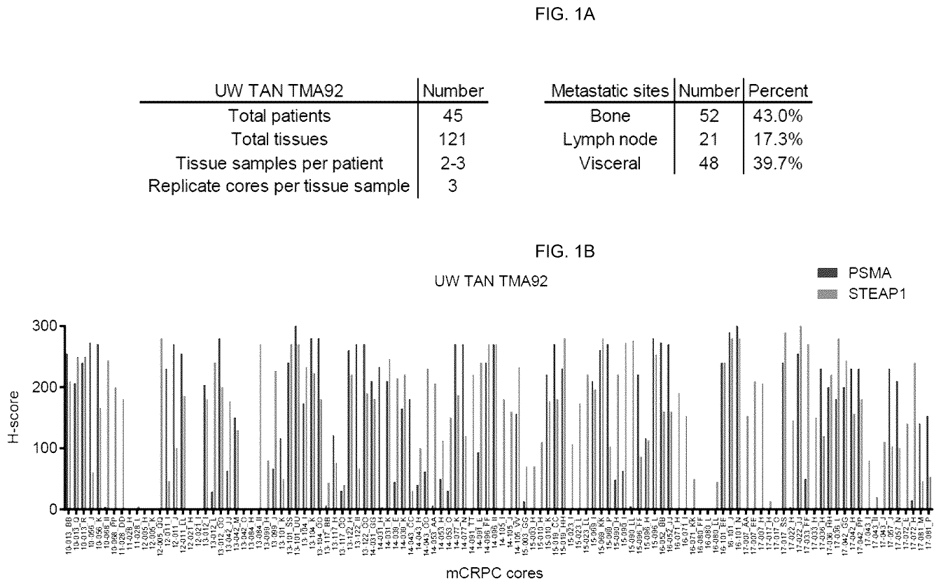

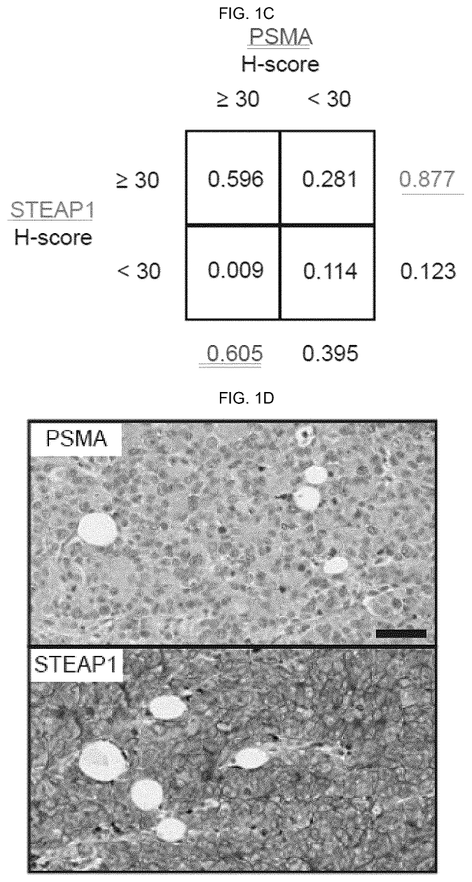

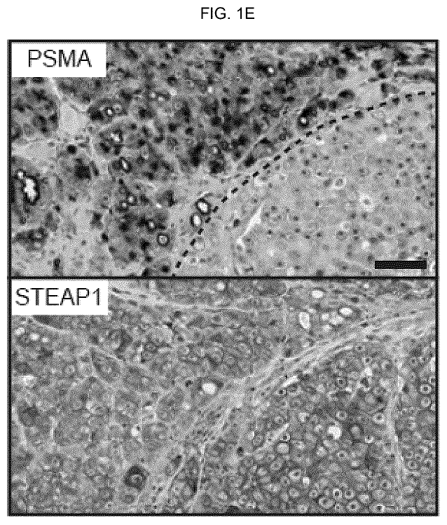

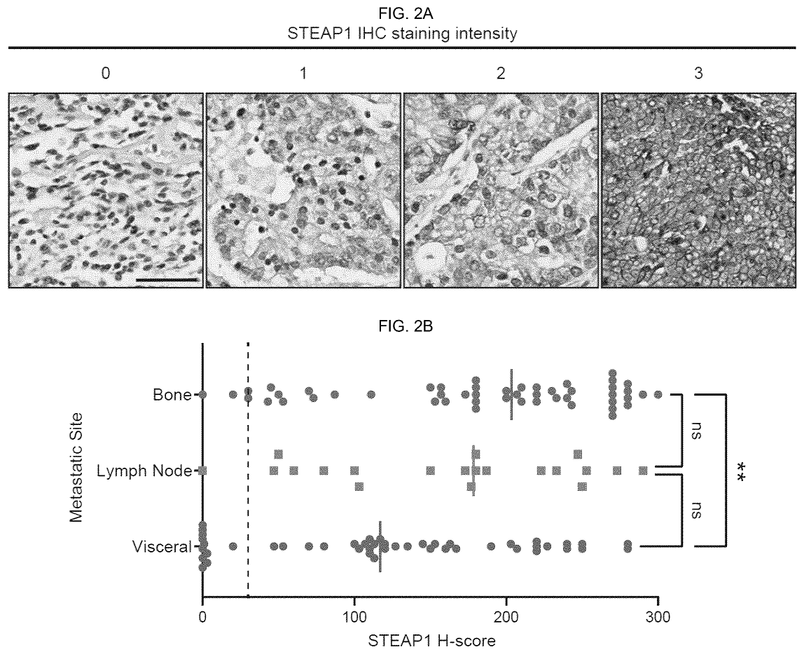

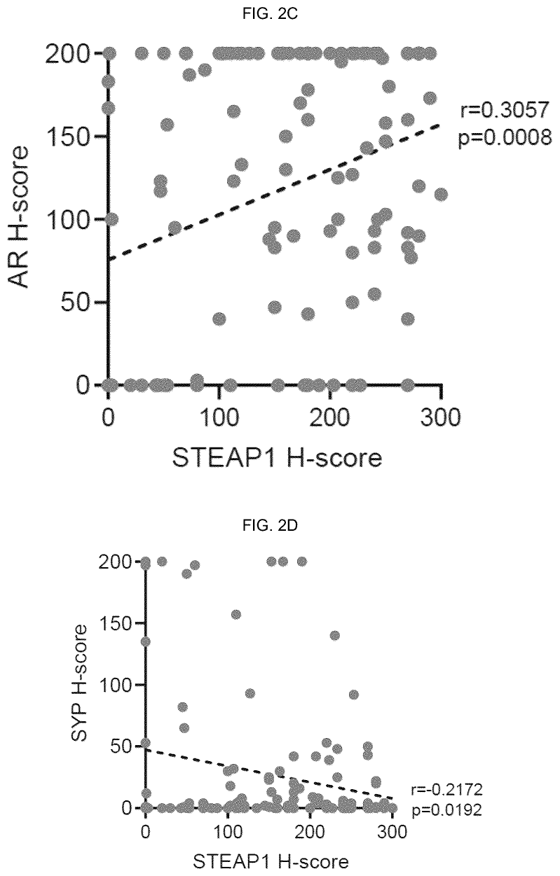

Results. STEAP1 is broadly expressed in lethal metastatic castration-resistant prostate cancer (mCRPC) tissues. First, the pattern and extent of STEAP1 expression relative to prostate-specific membrane antigen (PSMA) in advanced prostate was evaluated. Immunohistochemical (IHC) staining was performed on a duplicate set of tissue microarrays consisting of 121 cores of metastatic tissues collected from 45 lethal mCRPC patients at rapid autopsy between the years 2010 and 2017 by the University of Washington Tumor Acquisition Necropsy Program (Roudier, M. P., et al. Human pathology 34, 646-653 (2003)) (FIG. 1A). Plasma membrane staining for STEAP1 and PSMA in each core was scored by a research pathologist and semiquantitative H-scores were determined based on the staining intensity (0, 1, 2, or 3, FIG. 2A) multiplied by the percentage of cancer cells staining at each intensity (FIG. 1B). Based on these results, a generalized linear mixed statistical model was used to determine that the odds of non-zero staining were 7.7-fold (95% CI 2.8 to 20.8, p<0.001) higher for STEAP1 than for PSMA. By implementing a minimal staining threshold with an H-score cut-off of 30, it was found that 87.7% of evaluable matched cores (100 of 114) demonstrated staining for STEAP1 compared to 60.5% (69 of 114) for PSMA (FIG. 1C). In addition, 28.1% of cores (32 of 114) showed STEAP1 but not PSMA staining (FIG. 1D) whereas 0.9% of cores (one of 114) exhibited PSMA but not STEAP1 staining. Several cases with heterogeneous expression of PSMA within cores (FIG. 1E) were also observed which is consistent with a recent report of intratumoral PSMA heterogeneity in mCRPC biopsies (Paschalis, et al. European urology (2019)).

STEAP1 staining based on the minimal staining threshold was identified in 96% (48 of 50) of bone metastases, 95% (19 of 20) of lymph node metastases, and 76.6% (36 of 47) of visceral metastases (FIG. 2B). No difference in STEAP1 staining intensity was observed between bone and lymph node or lymph node and visceral metastatic sites. However, bone metastases demonstrated a higher STEAP1 H-score than visceral metastases (183.6 vs. 121.9, p=0.0018). A positive Pearson correlation (r=0.3057, 95% CI 0.1314 to 0.4616, p<0.001) was identified between the expression of STEAP1 and androgen receptor (AR) in cases represented on the tissue microarray (FIG. 2C) which was expected given that STEAP1 is an androgen-regulated gene (Gomes, I. M., et al., The Prostate 73, 605-613 (2013); and Sharp, A., et al., The Journal of clinical investigation 129, 192-208 (2019)). In contrast, a negative correlation (r=−0.2172, 95% Cl −0.3843 to −0.03628, p=0.0192) was appreciated between the expression of STEAP1 and the neuroendocrine differentiation marker synaptophysin (SYP) (FIG. 2D). These findings suggest that, like PSMA (Bakht, et al., Endocrine-related cancer 26, 131-146 (2018)), STEAP1 expression may be lost with neuroendocrine transdifferentiation of prostate cancer.

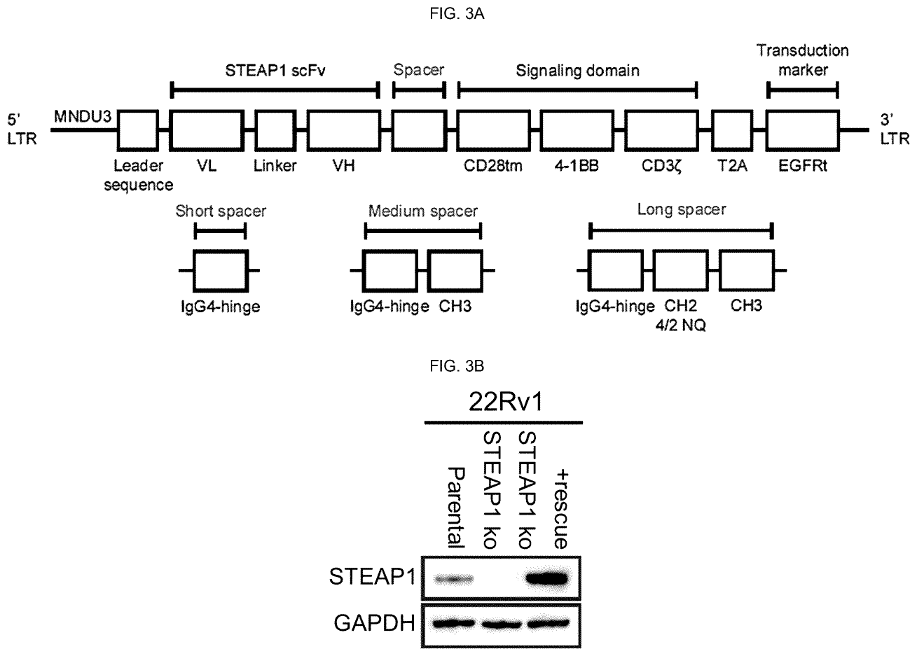

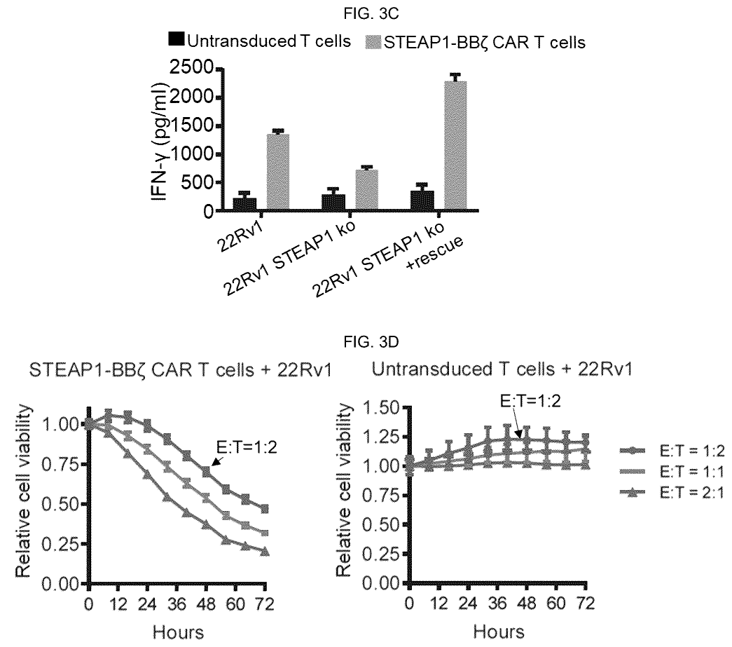

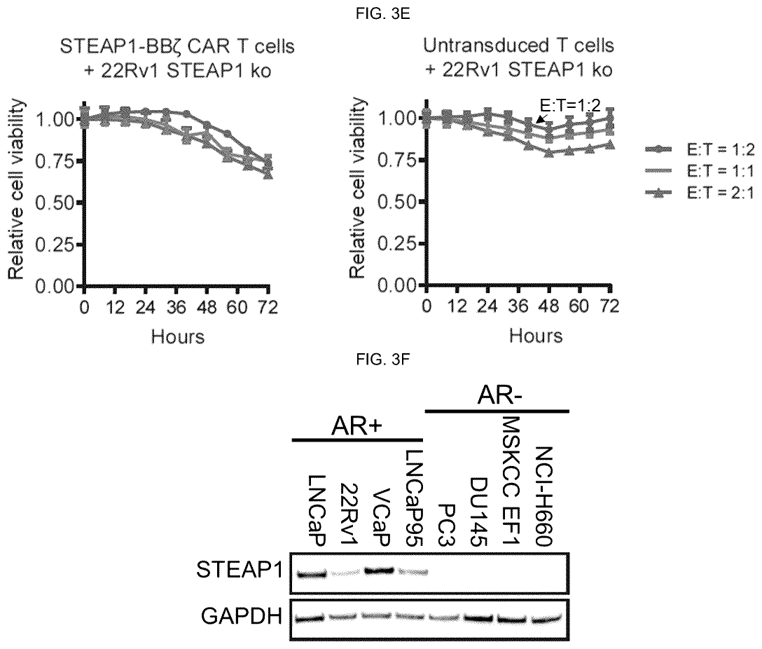

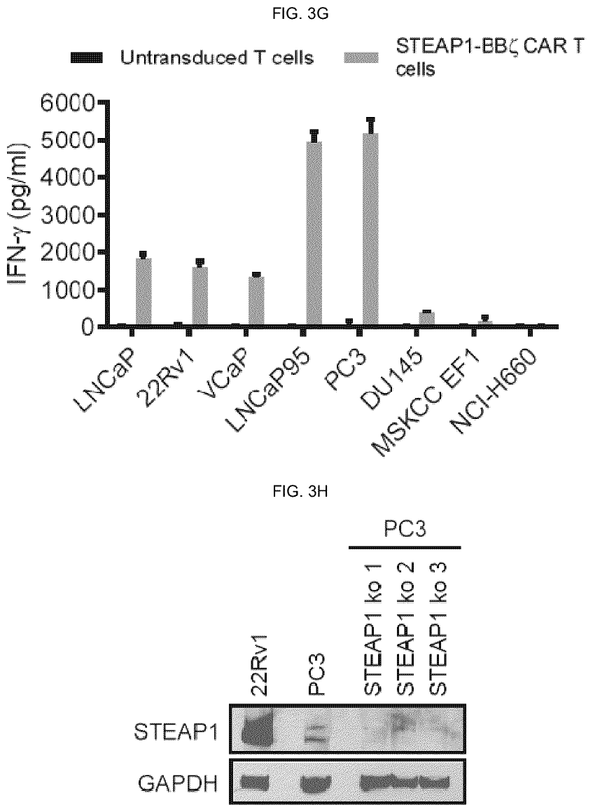

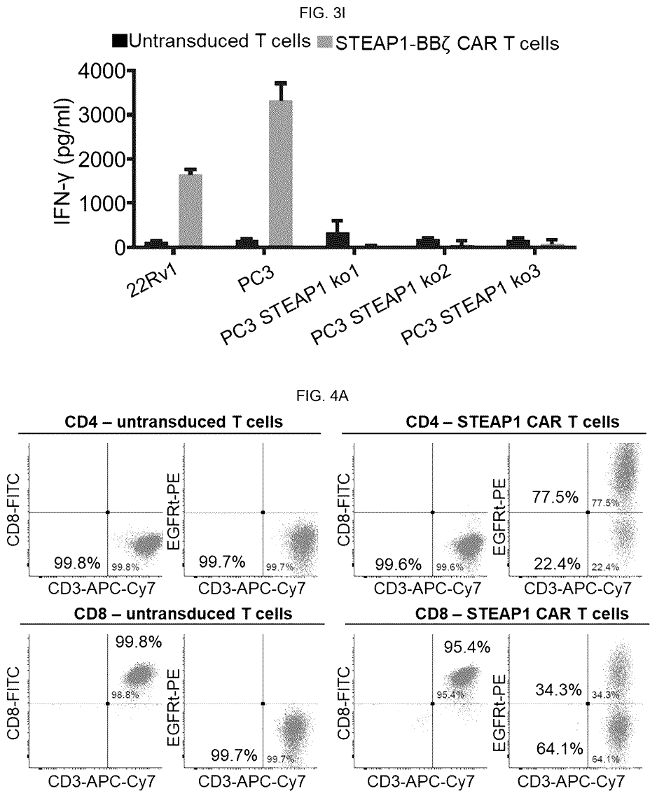

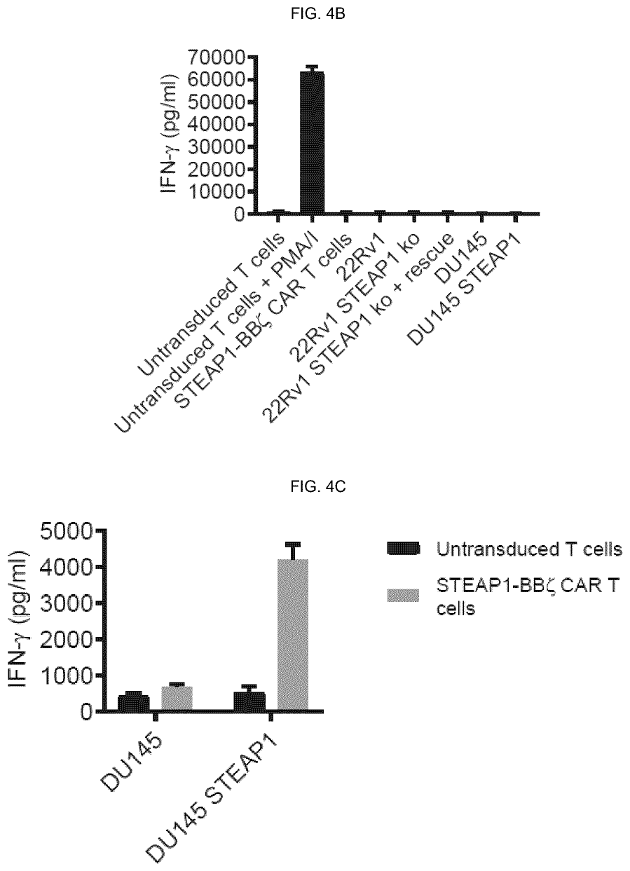

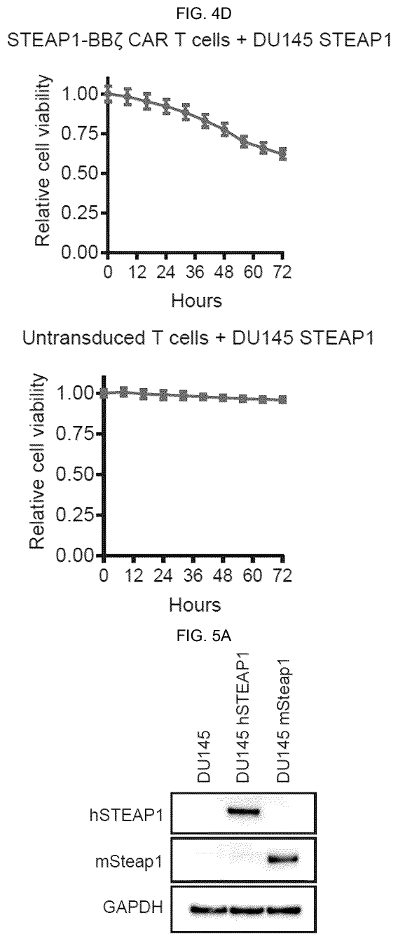

Development of a potent, antigen-specific STEAP1 CAR. Having affirmed the rationale for targeting STEAP1 in late-stage mCRPC because of its widespread expression, a lentiviral STEAP1-specific 2nd generation CAR was engineered. A 4-1BB costimulatory domain was favored due to its association with T cell memory formation and prolonged persistence (Salter, et al., Science signaling 11 (2018)) and a CD28 transmembrane domain was introduced as this has been shown to reduce the antigen-threshold for second-generation 4-1BB CAR T cell activation (Majzner, et al., Cancer discovery 10, 702-723 (2020)). The fully humanized single-chain variable fragment (scFv) derived from vandortuzumab vedotin (DSTP3086S), an antibody-drug conjugate targeting STEAP whose development was discontinued after a phase I/II trial due to an unfavorable therapeutic window likely from payload deconjugation was incorporated. This scFv is a humanized variant of the murine monoclonal antibody (mAb 120.545) originally developed by Agensys, Inc. that demonstrates 1 nM affinity in cell-based binding assays (Challita-Eid, P. M., et al., Cancer research 67, 5798-5805 (2007)). To potentially tune CAR activity, three different hinge/spacer lengths were implemented, including short (IgG4 hinge), medium (IgG4 hinge-CH3), and long (IgG4 hinge-CH2-CH3). The long spacer was engineered with previously described 4/2-NQ mutations (Hudecek, et al., Cancer immunology research 3, 125-135 (2015)) in the CH2 domain to prevent Fc-gamma receptor binding and activation-induced cell death that occurs with the adoptive transfer of long spacer CAR T cells into immunodeficient mice. The three candidate CAR were each cloned into a lentiviral vector (FIG. 3A) that also co-expresses truncated epidermal growth factor receptor (EGFRt) as a transduction marker. Lentiviruses were generated and used to transduce human CD4 and CD8 T cells enriched from human donor peripheral blood mononuclear cells (PBMCs) collected from pheresis. Expanded CD4 and CD8 CAR T cells were immunophenotyped (FIG. 4A) and reconstituted into cell products of a defined composition with a normal CD4/CD8 ratio to evaluate their functional activities.

To control for STEAP1 expression in an isogenic manner, the focus was the 22Rv1 human prostate cancer cell line that demonstrates native STEAP1 expression. STEAP1 knockout (ko) by CRISPR/Cas9 genome editing was performed. A STEAP1 rescue line was then generated from the 22Rv1 STEAP1 ko by transduction with a STEAP1 expressing lentivirus (FIG. 3B). These lines were then used initially to screen the short, medium, and long spacer STEAP1 CAR T cells in co-culture assays with a readout of interferon-γ (IFN-γ) release as an indicator of T cell activation. The long spacer STEAP1 CAR T cells (referred to as STEAP1-BBζ CAR T cells) demonstrated antigen-specific pattern of IFN-γ release (FIG. 3C, FIG. 4B). Further, STEAP1-BBζ CAR T cells showed substantial dose-dependent cytolysis of 22Rv1 cells compared to untransduced T cells (FIG. 3D) and demonstrated relative sparing of 22Rv1 STEAP1 ko cells (FIG. 3E). Similar studies were then performed in the DU145 human prostate cancer cell line that lacks native STEAP1 expression but was engineered to express STEAP1 (DU145 STEAP1) by lentiviral transduction. In this setting, STEAP1-BBζ CAR T cell activation was observed in co-cultures with DU145 STEAP1 cells and not the parental DU145 cells (FIG. 4C). Cytolytic activity was observed with STEAP1-BBζ CAR T cells and not untransduced T cells in co-cultures with DU145 STEAP1 cells (FIG. 4D).

A larger panel of human prostate cancer cell lines was subsequently analyzed to characterize their native STEAP1 expression by immunoblot analysis. The cell lines with known AR expression/activity (LNCaP, 22Rv1, VCaP, and LNCaP95) showed varying levels of STEAP1 expression while the AR-null cell lines (PC3, DU145, MSKCC EF1, and NCI-H660) did not appear to express detectable levels of STEAP1 (FIG. 3F). Co-cultures of STEAP1-BBζ CAR T were performed with these cell lines to further validate their antigen-specific activation based on IFN-γ release (FIG. 3G). However, a discordant finding was identified in that the PC3 line, which showed no apparent STEAP1 expression (FIG. 3F), induced substantial activation of STEAP1-BBζ CAR T cells. Prior literature suggested that STEAP1 is expressed in the PC3 cell line at low levels (Gomes, I. M., et al., Genes & cancer 5, 142-151 (2014)). Indeed, when the immunoblots were exposed significantly longer, a band suggesting the presence of very low expression of STEAP1 (FIG. 3H) was seen. To confirm whether the STEAP1-BBζ CAR T cell activation was due to this minor STEAP1 expression in PC3, three PC3 STEAP1 ko sublines (FIG. 3G) were generated and co-cultures were again performed with STEAP1-BBζ CAR T cells. STEAP1 ko in the PC3 line led to the abrogation of STEAP1-BBζ CAR T cell activation (FIG. 3I), further validating specificity and providing evidence of the sensitivity of STEAP1-BBζ CAR T cells to low antigen density conditions.

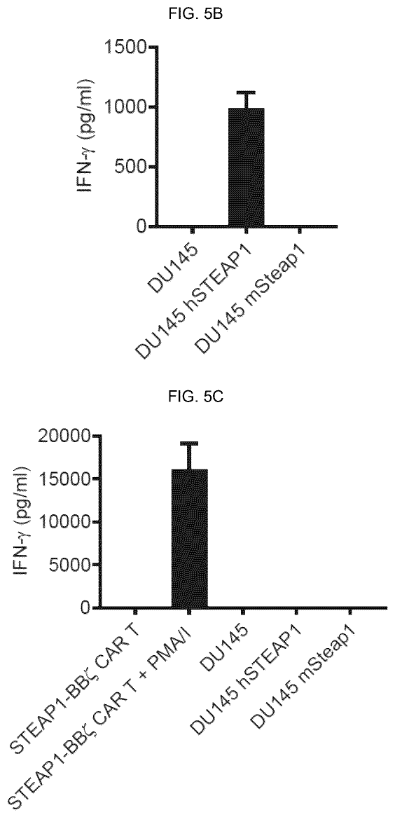

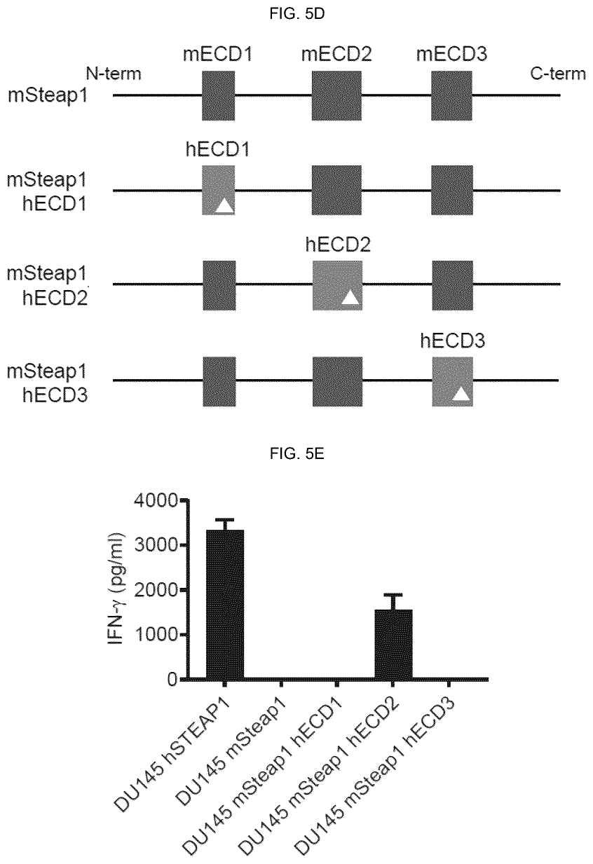

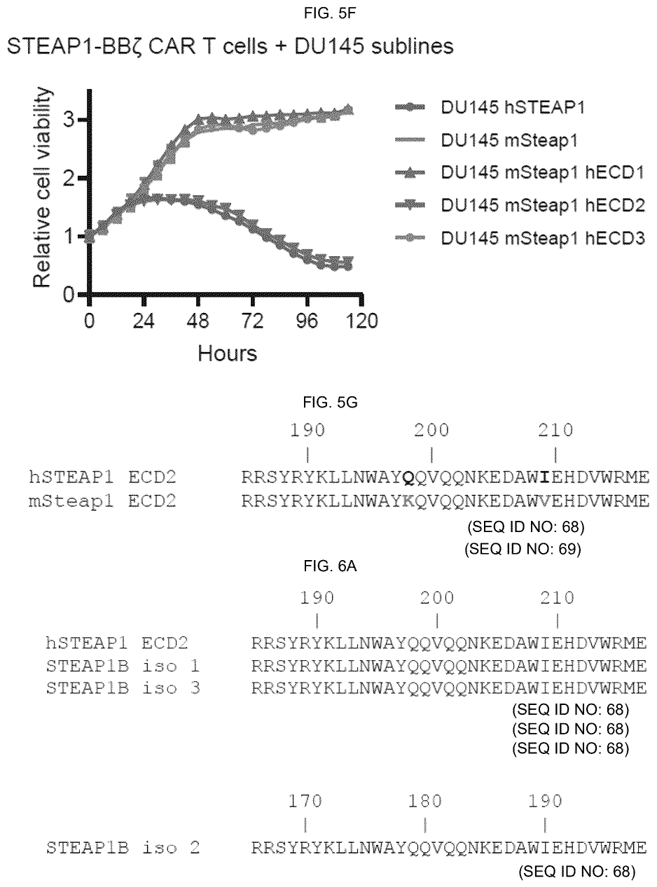

Functional determination of the cross-reactivity of STEAP1-BBζ CAR with mouse Steap1 and human STEAP1B. Consistent with the anti-human specificity of the scFv derived from mAb 120.545, STEAP1-BBζ CAR T cells did not demonstrate cross reactivity with mouse Steap1 (FIGS. 5A-5C). However, this was used as an opportunity to individually reconstitute the three human STEAP1 extracellular domains (ECDs) onto mouse Steap1 (FIG. 5D) to determine which ECDs are critical for epitope recognition by STEAP1-BBζ CAR T cells. Co-culture experiments were performed with STEAP1-BBζ CAR T cells and DU145 cells engineered to express mouse Steap1 with individual replacement of mouse ECDs with human ECDs. It was found that human STEAP1 ECD2 but not ECD1 or ECD3 was associated with STEAP1-BBζ CAR T cell activation (FIG. 5E) and target cell cytolysis (FIG. 5F). Interestingly, the human STEAP1 and mouse Steap1 ECD2 demonstrate 93.9% (31/33 amino acids) homology (FIG. 5G), indicating that Q198 and/or 1209 of human STEAP1 are critical to productive recognition by STEAP1-BBζ CAR T cells.

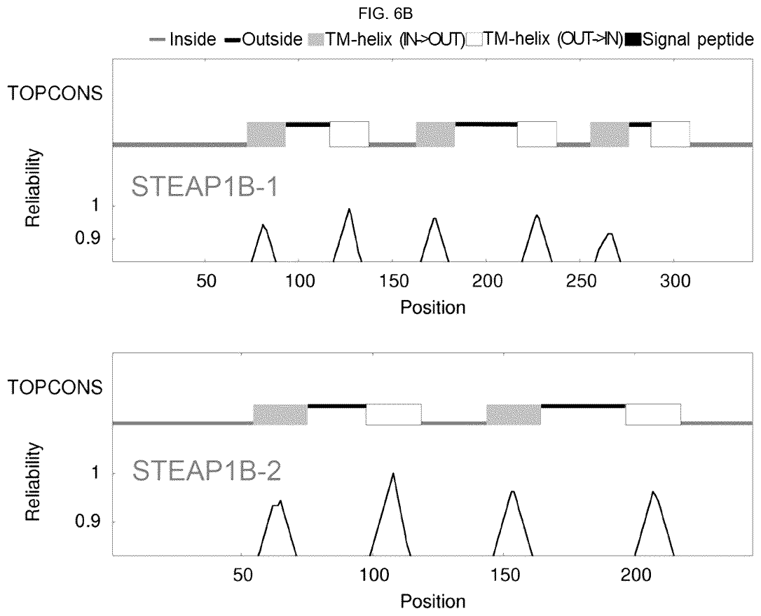

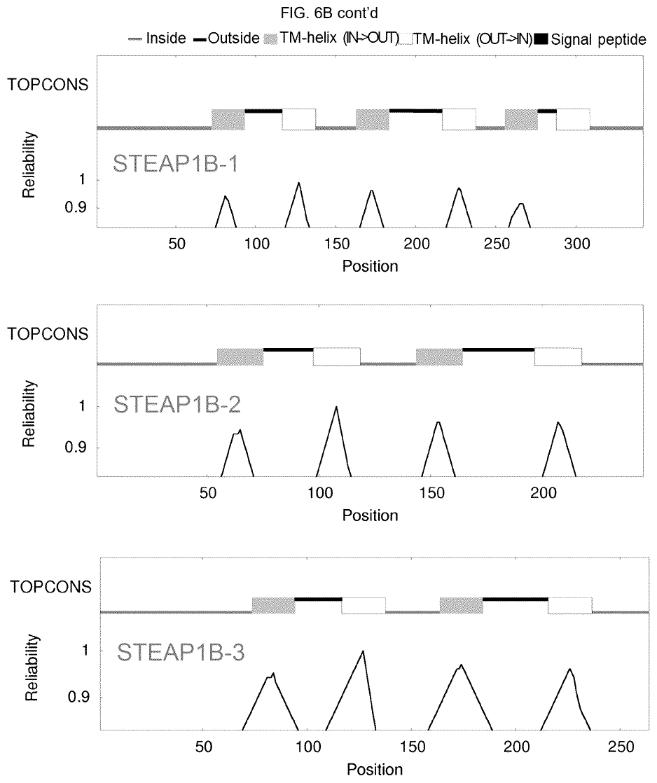

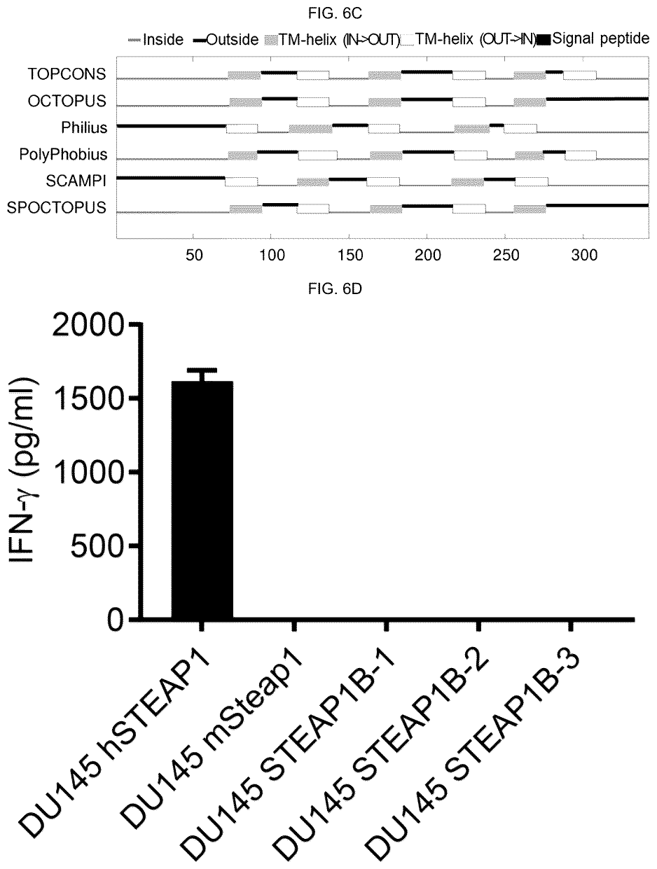

Of the human STEAP family of proteins, STEAP1B has the greatest homology to STEAP1 (Gomes, I. M., et al., Genes & cancer 5, 142-151 (2014)). Three STEAP1B transcripts have been identified, of which all demonstrate complete conservation of the amino acid sequence of human STEAP1 ECD2 (FIG. 6A). The consensus membrane topology prediction algorithm TOPCONS (Bernsel, A., et al., Nucleic acids research 37, W465-W468 (2009)) projected these sequences as being extracellular in the three STEAP1B protein isoforms (FIG. 6B) albeit with low reliability scores due to a lack of consensus between models (FIG. 6C). Prior analysis using a hidden Markov model had also suggested that this sequence could be intracellular rather than extracellular in STEAP1B protein isoforms 1 and 2 (Gomes, I. M., et al., Genes & cancer 5, 142-151 (2014)). However, the crystal structure of STEAP1B has yet to be resolved to directly substantiate these predictions.

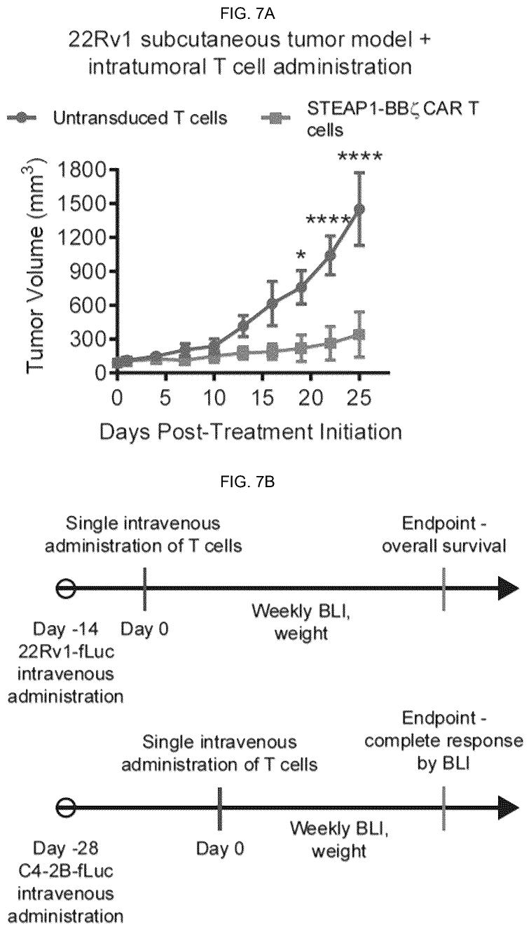

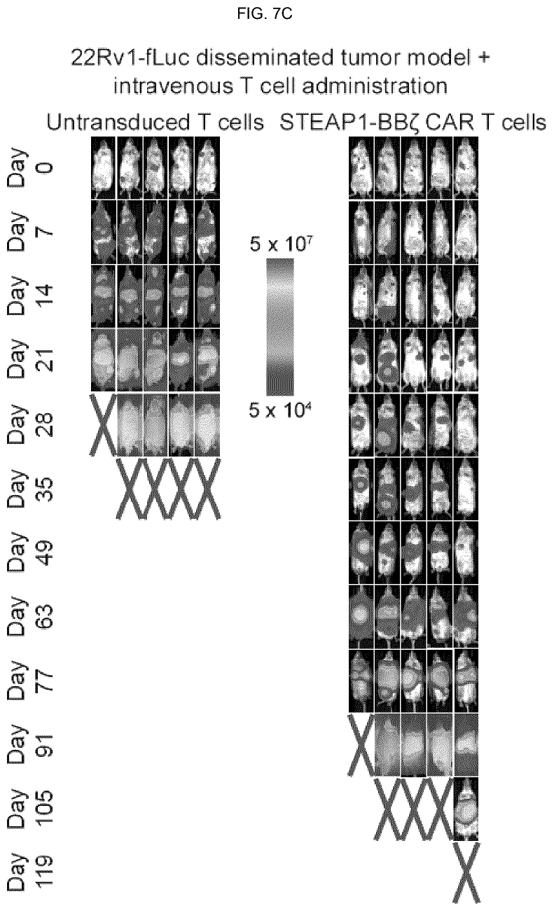

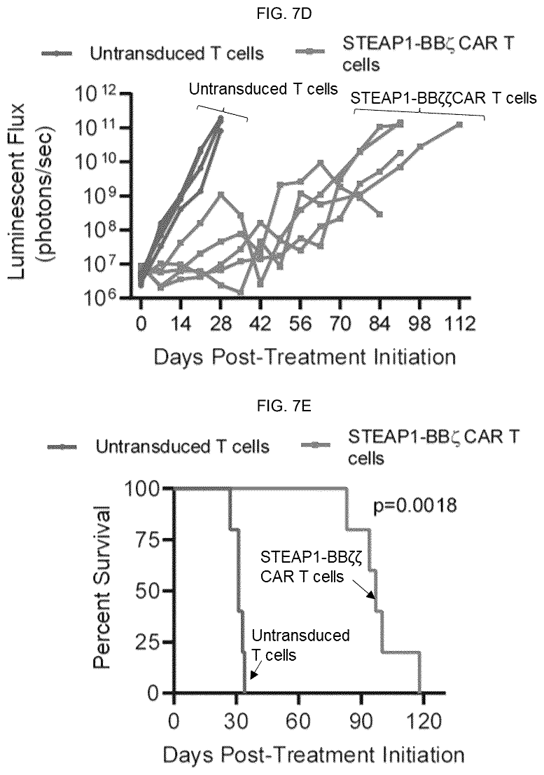

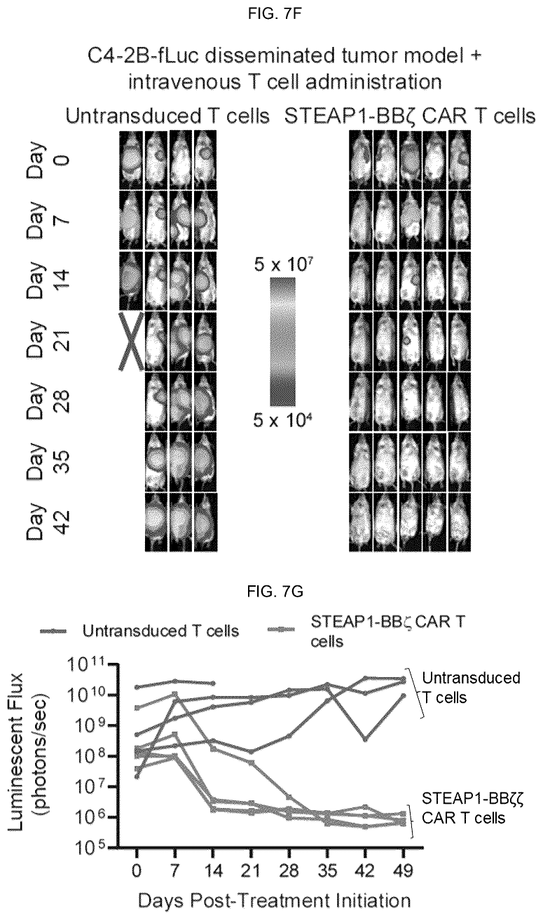

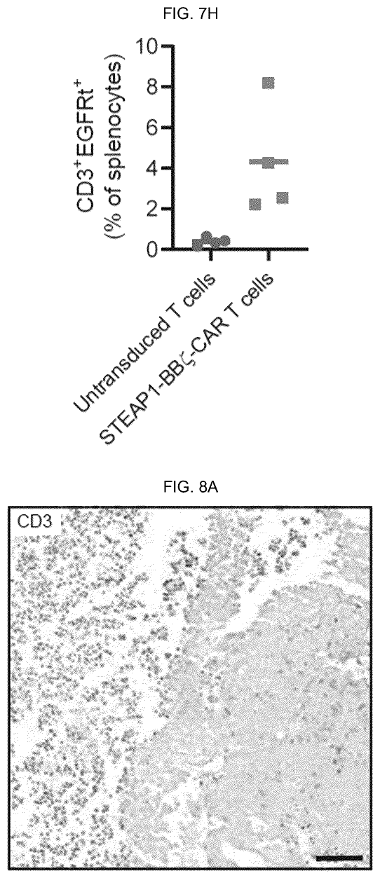

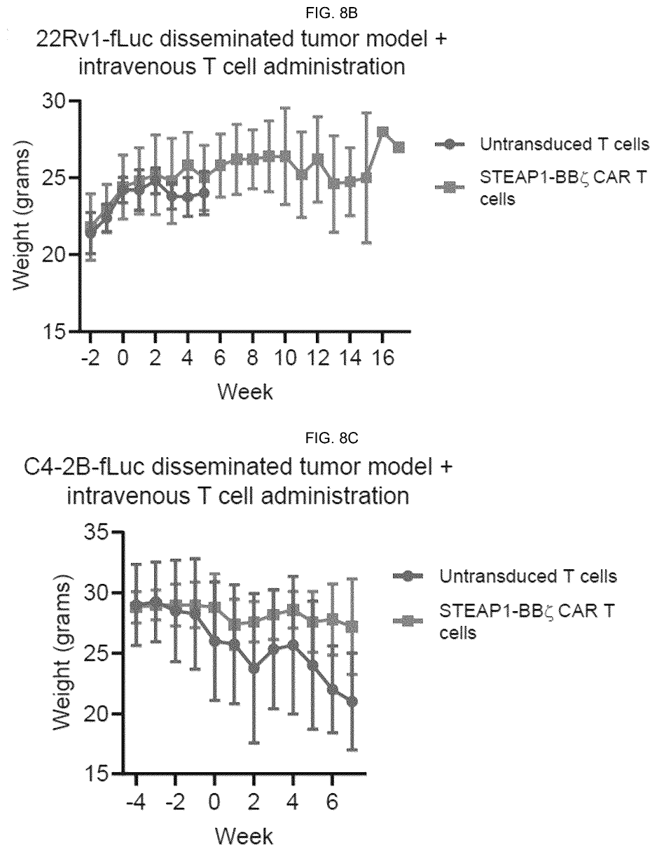

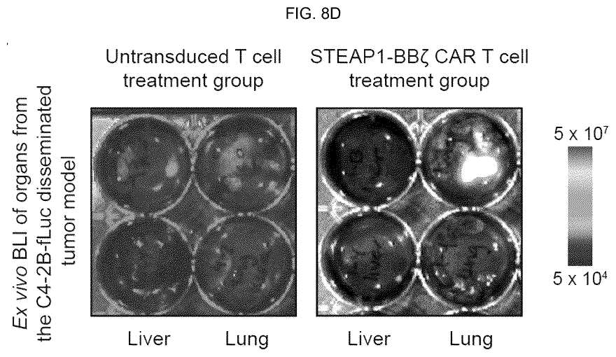

STEAP1-BBζ CAR T cells demonstrate substantial antitumor effects in disseminated prostate cancer models with native STEAP1 expression established in immunodeficient mice. As an initial screen for in vivo antitumor activity, 22Rv1 subcutaneous xenograft tumors were established in male NOD scid gamma (NSG) mice. When tumors grew to 100 mm³, mice were treated with a single intratumoral injection of either 5×10⁶ untransduced T cells or STEAP1-BBζ CAR T cells. Intratumoral treatment with STEAP1-BBζ CAR T cells was associated with significant tumor growth inhibition that was statistically significant by day 16 post-treatment (FIG. 7A). Mice were sacrificed on day 25 and residual tumors from mice treated with STEAP1-BBζ CAR T cells showed large areas of necrotic debris and regions of viable tumor were infiltrated with CD3+ STEAP1-BBζ CAR T cells (FIG. 8A).

22Rv1 cells were transduced with lentivirus to enforce firefly luciferase (fLuc) expression and 10⁶ 22Rv1-fLuc cells were injected into the tail veins of male NSG mice. Metastatic colonization was visualized by live bioluminescence imaging (BLI) after two weeks, at which point mice were treated with a single intravenous injection of either 5×10⁶ untransduced T cells or STEAP1-BBζ CAR T cells (FIG. 7B). Serial BLI revealed rapid disease progression in mice treated with untransduced T cells while those who received STEAP1-BBζ CAR T cells demonstrated a significant delay in tumor progression (FIGS. 7C, 7D) and extension of survival (97 days versus 31 days, p=0.0018 by log-rank test, FIG. 7E). There was no significant difference in mouse weights between treatment arms (FIG. 8B).

Male NSG mice were also inoculated with C4-2B-fLuc cells by tail vein injection. C4-2B is a castration-resistant subline of LNCaP (Chen, M. E., et al., The Journal of biological chemistry 273, 17618-17625 (1998)) with growth kinetics more in line with typical prostate cancer. Four weeks after injection, metastatic colonization was confirmed by BLI and mice were treated with single intravenous injection of either 5×10⁶ untransduced T cells or STEAP1-BBζ CAR T cells (FIG. 7B). Serial BLI showed a complete response in all mice who received STEAP1-BBζ CAR T cells within five weeks of treatment (FIGS. 7F, 7G). A trend of increasing weight loss was identified in the untransduced T cell treatment group (FIG. 8C) but this was not statistically significant likely due to the small sample size. Necropsy of mice treated with STEAP1-BBζ CAR T cells showed no macroscopic disease and ex vivo BLI of organs did not reveal any signal, suggesting that these mice were likely cured (FIG. 8D). Peripheral persistence of STEAP1-BBζ CAR T cells was identified at the end of the experiment based on the presence of detectable CD3+EGFRt+ splenocytes (FIG. 7H).

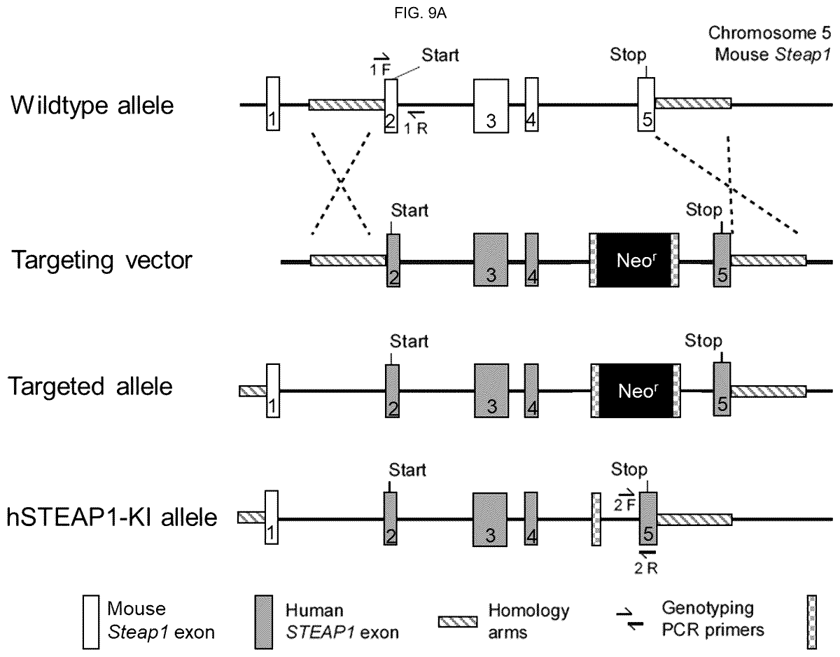

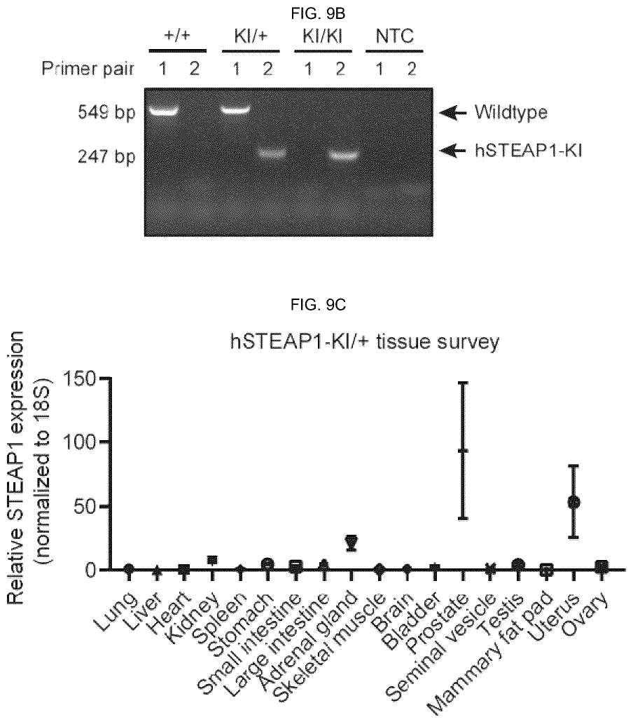

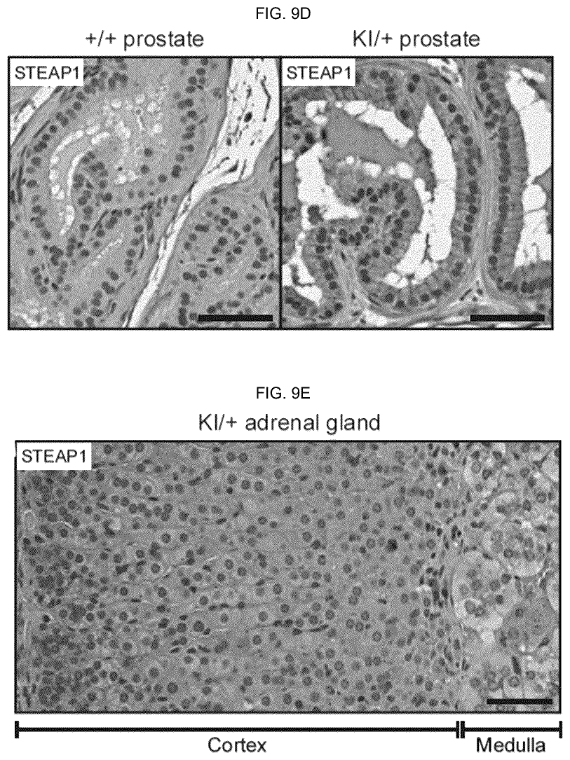

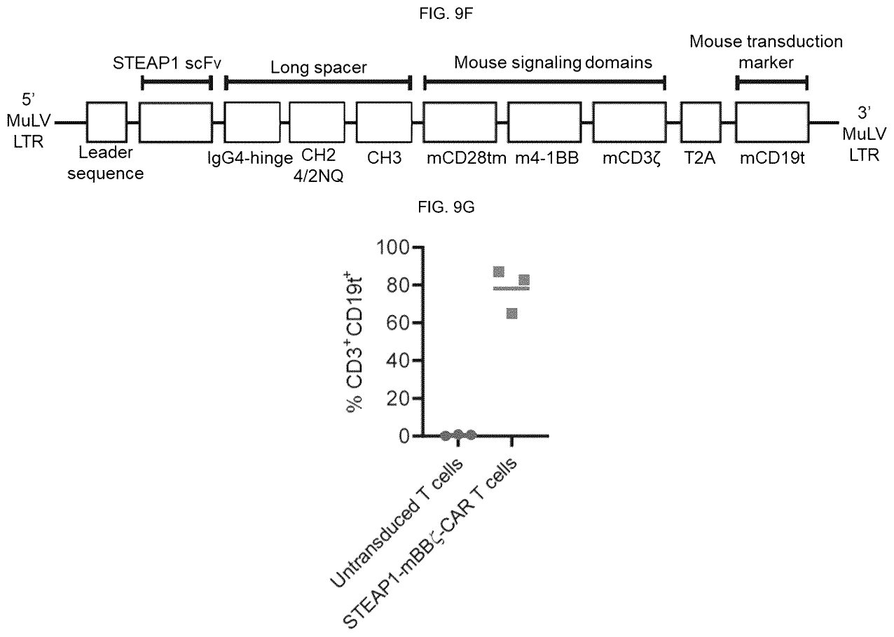

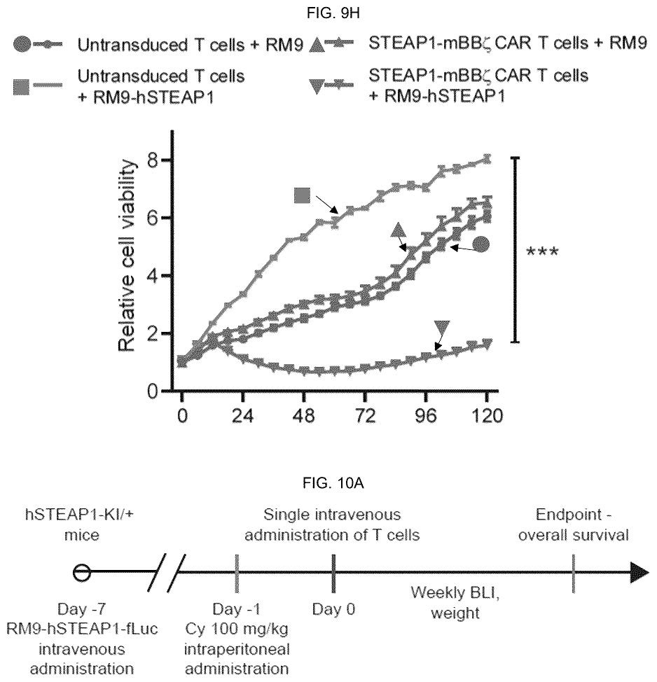

Mouse-in-mouse STEAP1 CAR T cell studies indicate safety and efficacy. The activation and cytolytic activity of STEAP1-BBζ CAR T cells observed in the very low STEAP1 antigen density context of the PC3 cell line presented concerns about the potential for on-target, off-tumor toxicities. To evaluate for potential toxicity in a tractable model organism, a human STEAP1 knock-in (hSTEAP1-KI) mouse was generated in which the human STEAP1 gene was knocked into the mouse Steap1 gene on the C57Bl/6 background (FIG. 9A). A mouse colony was established with genotyping performed by polymerase chain reaction (PCR) of tail DNA (FIG. 9B). Both homozygous and heterozygous hSTEAP1-KI mice exhibited no apparent phenotypic or reproductive abnormalities compared to wildtype littermates. A tissue survey for human STEAP1 expression based on quantitative reverse transcription PCR (qRT-PCR) was performed on male and female heterozygous hSTEAP1-KI (hSTEAP1-KI/+) mice and revealed greatest relative expression in the prostate, followed by the uterus and adrenal gland (FIG. 9C). Further in situ analysis by STEAP1 IHC of male hSTEAP1-KI/+ prostate and adrenal glands revealed human STEAP1 expression confined to luminal epithelial cells of the prostate (FIG. 9D) and expression in the adrenal cortex (FIG. 9E).

A murinized version of the STEAP1 CAR, called STEAP1-mBBζ CAR, in which the scFv and IgG4 hinge-CH2-CH3 spacer were retained but the CD28 transmembrane domain, 4-1BB costimulatory domain, and CD3ζ activation domain were replaced with their mouse orthologs was cloned into a gammaretroviral construct (FIG. 9F). In addition, the transduction marker EGFRt was replaced with a truncated mouse CD19 (mCD19t) to minimize potential immunogenicity. The efficient retroviral transduction of T cells enriched from mouse splenocytes (FIG. 9G) and the capacity of mouse STEAP1-mBBζ CAR T cells to induce cytolysis of the RM9 mouse prostate cancer cell line engineered to express human STEAP1 (RM9-hSTEAP1) by lentiviral transduction (FIG. 9H) was confirmed.

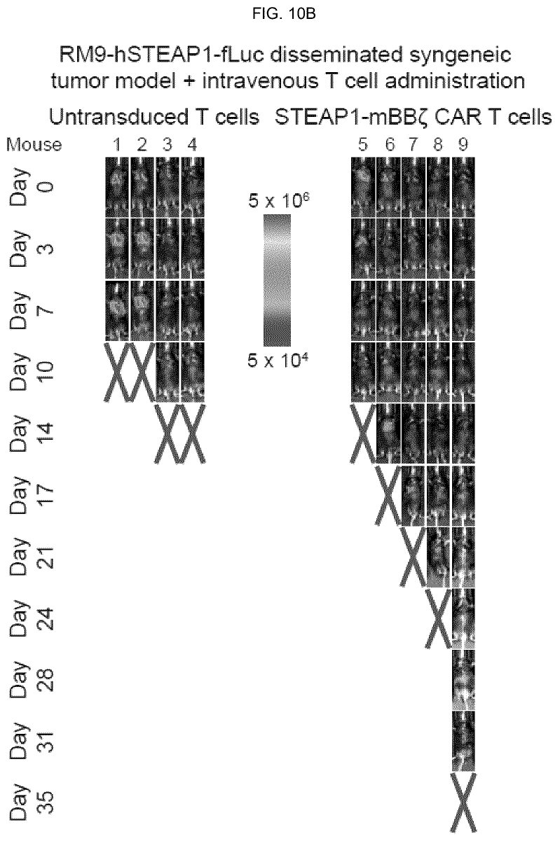

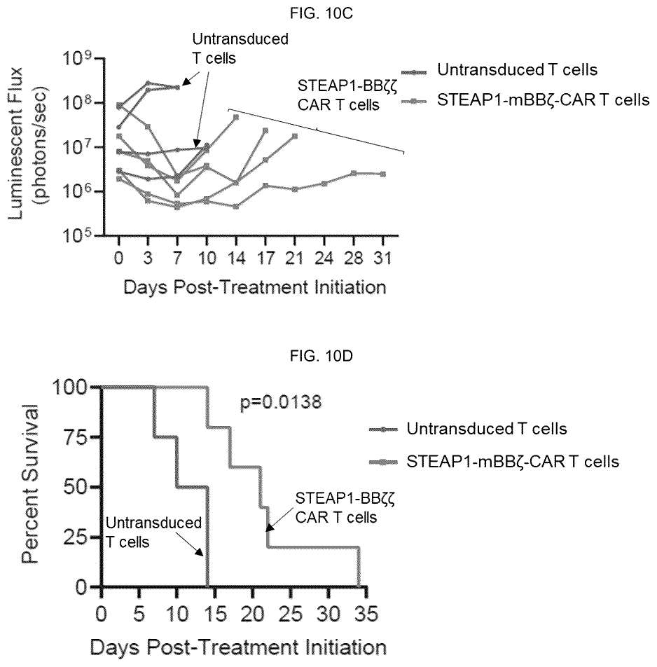

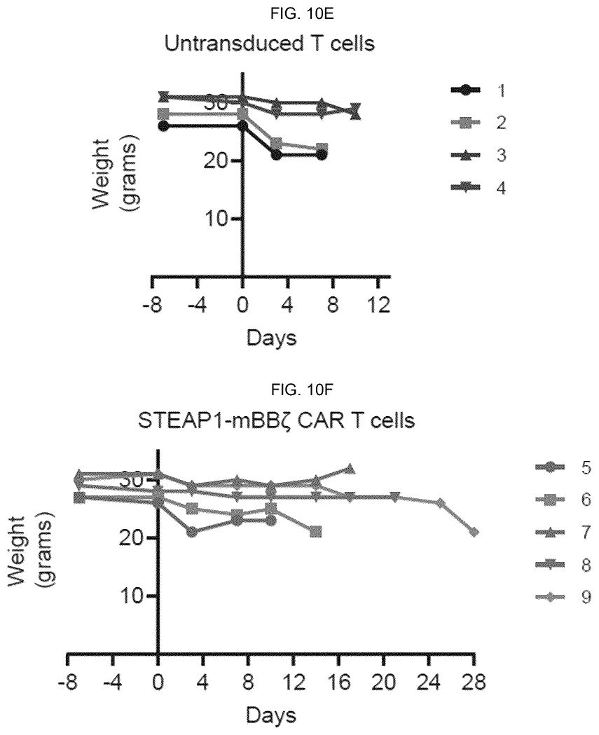

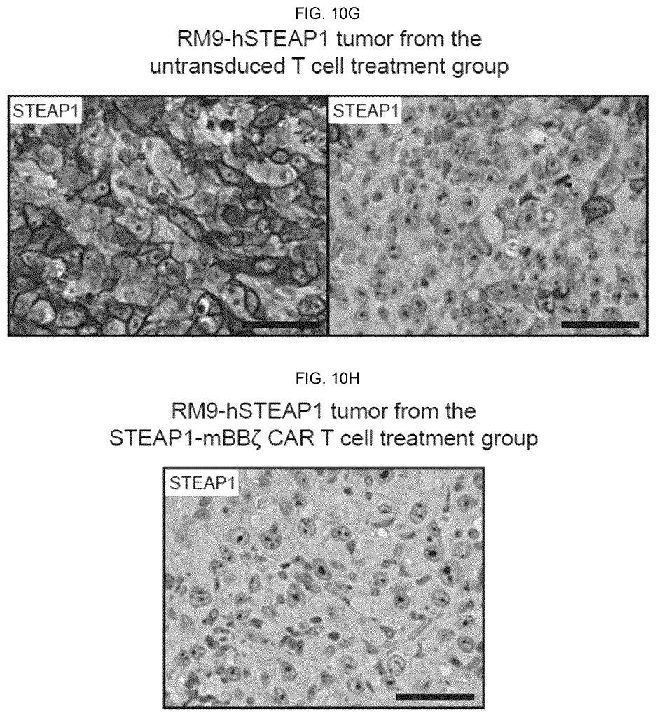

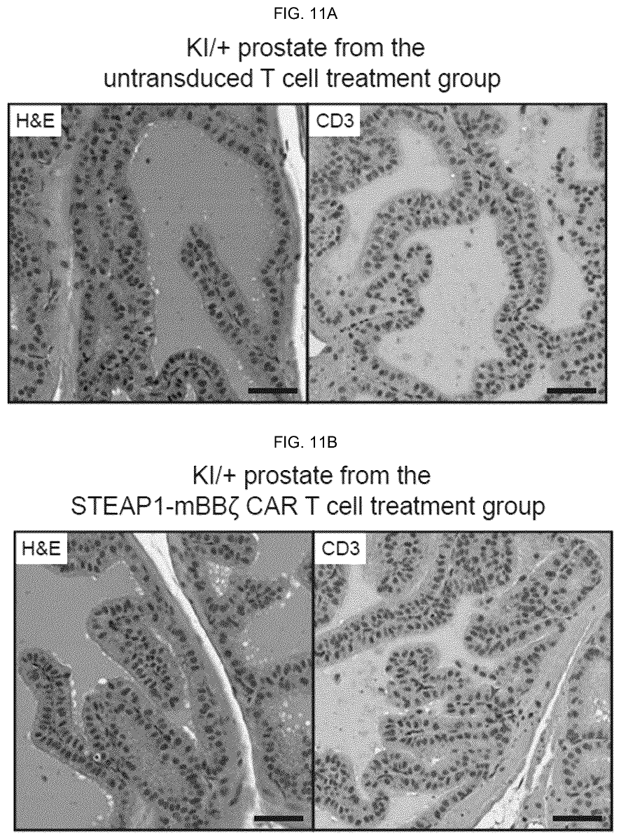

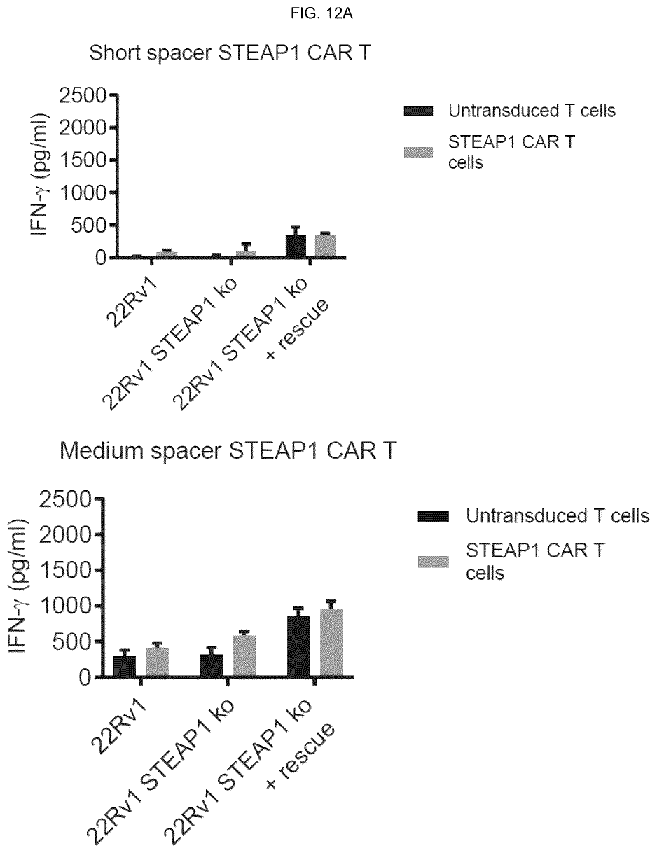

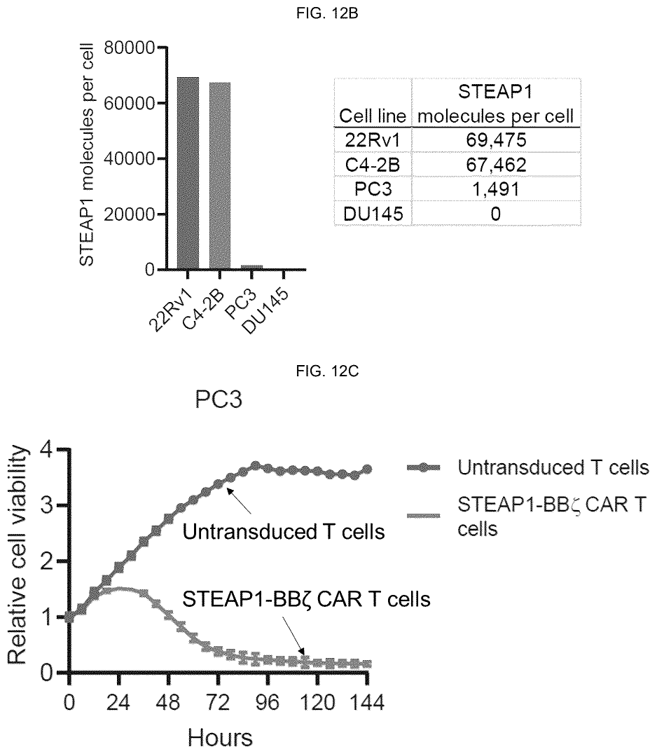

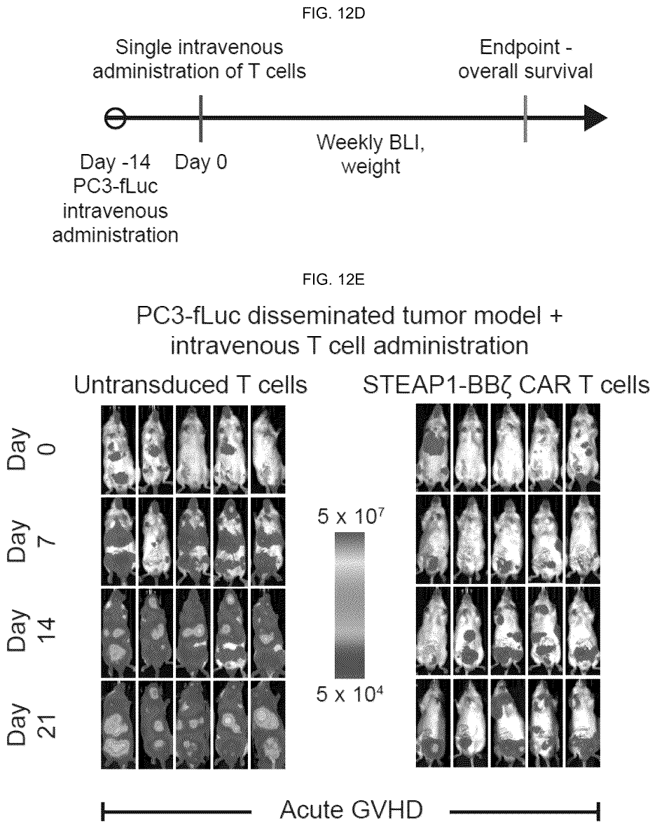

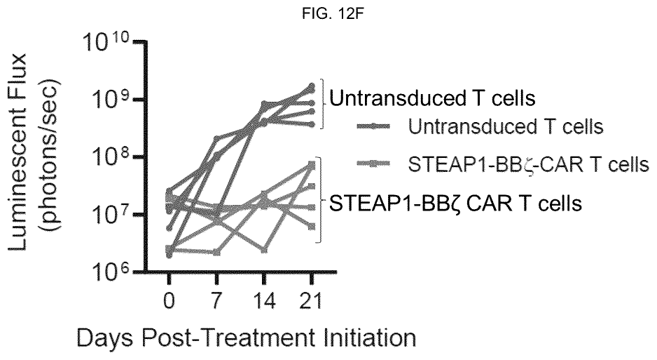

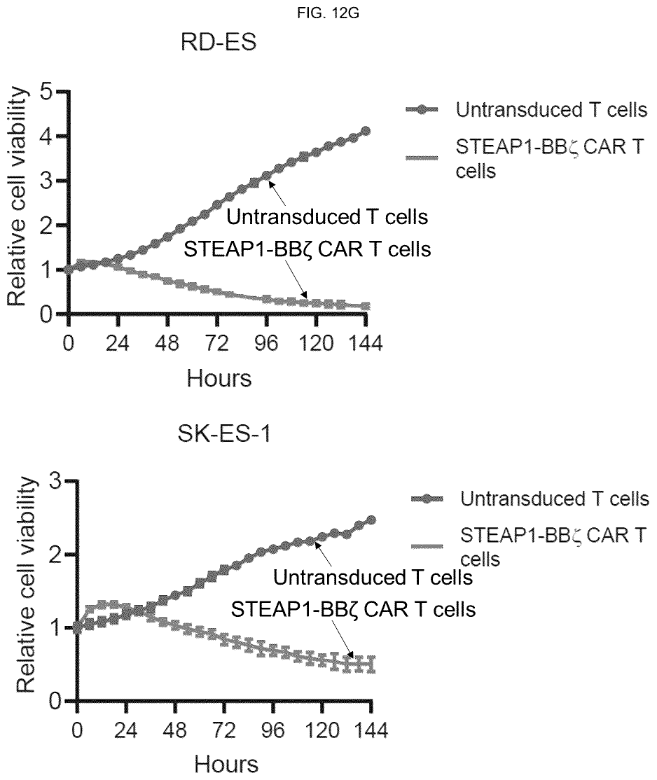

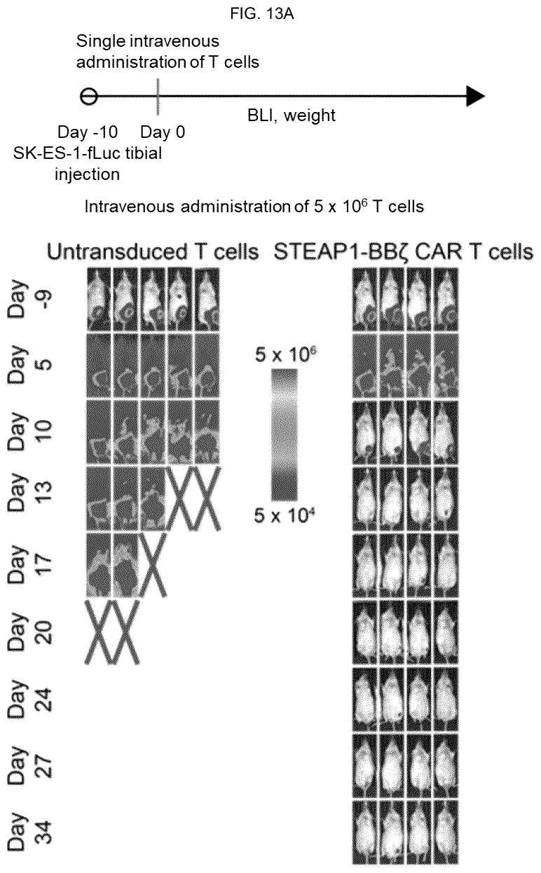

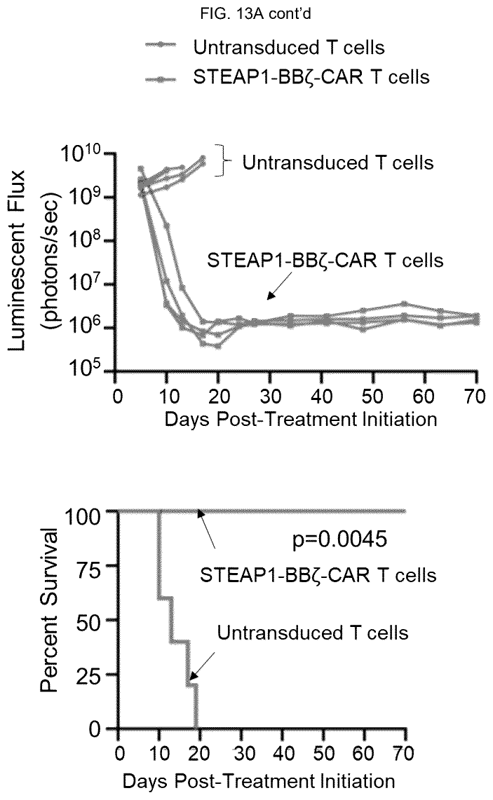

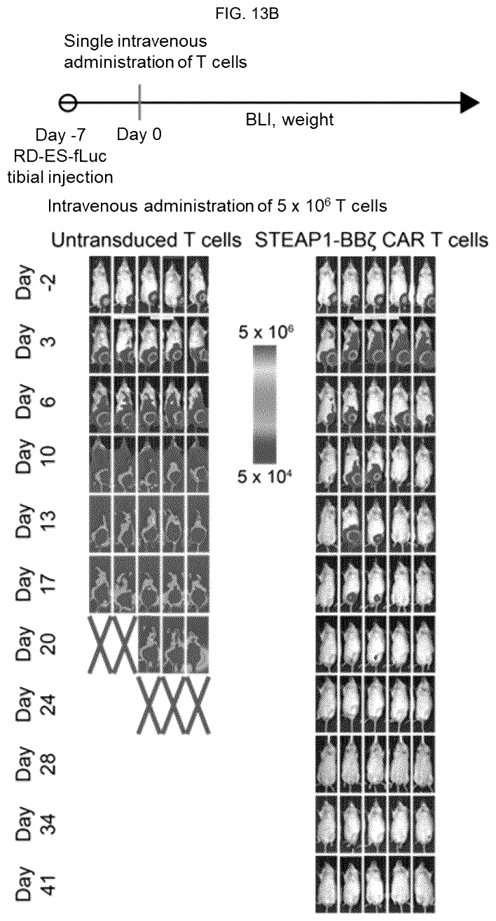

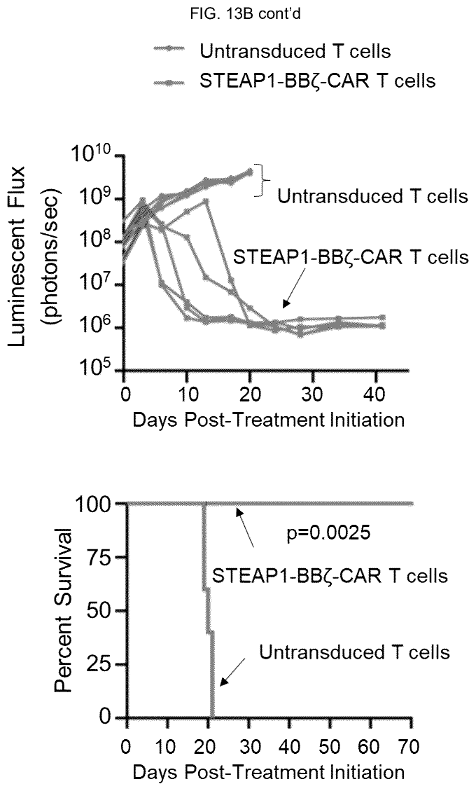

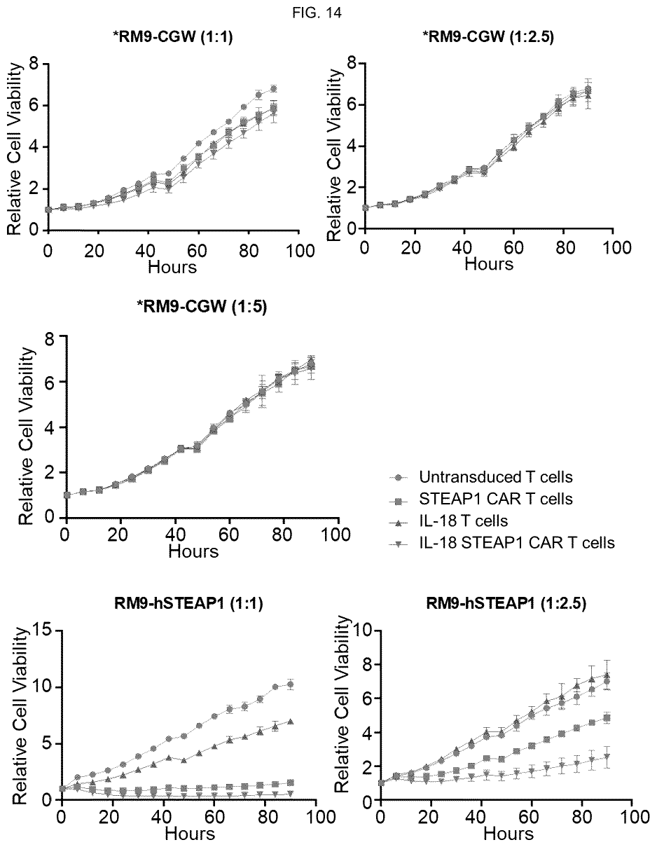

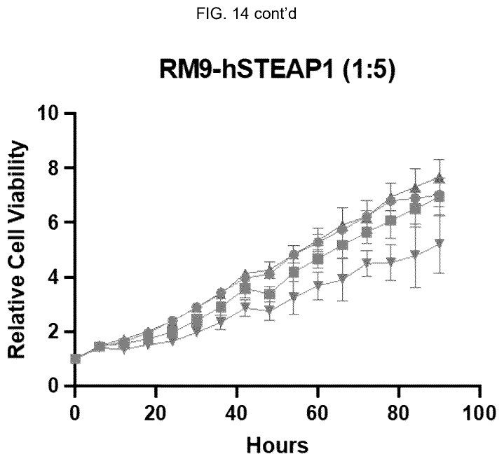

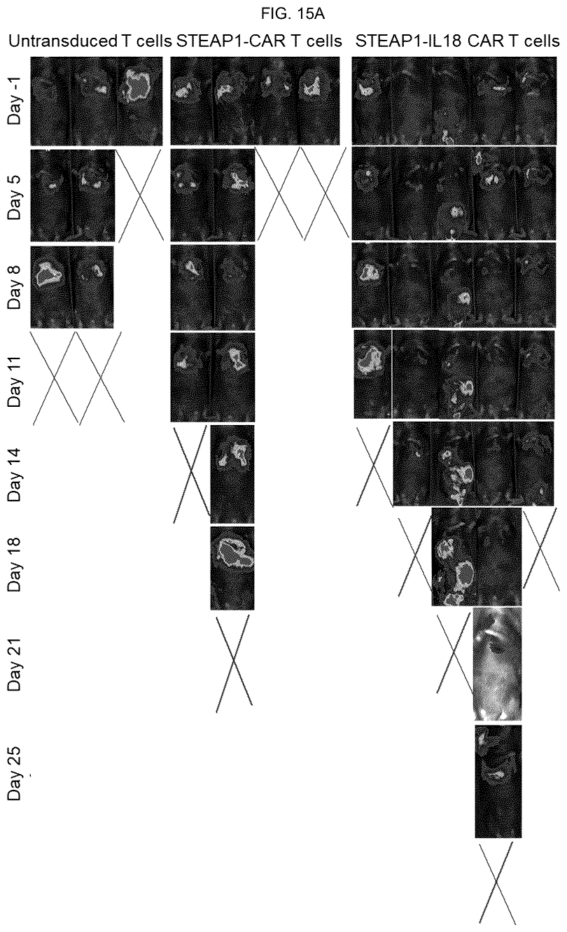

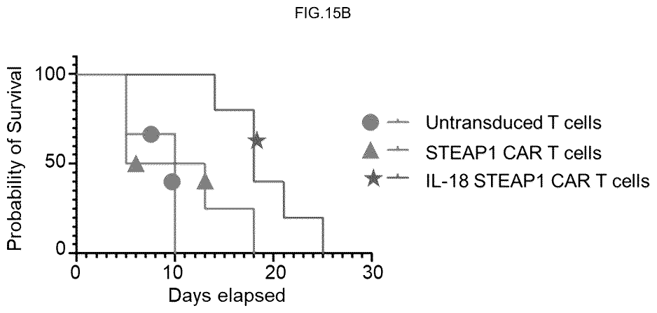

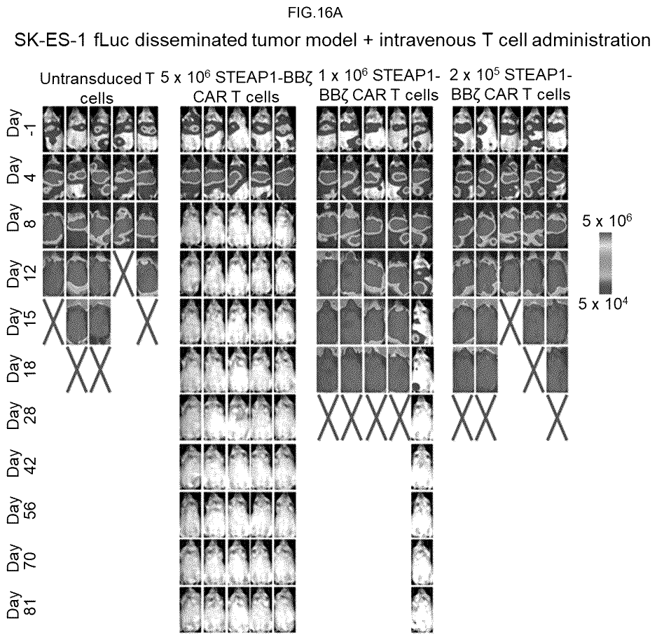

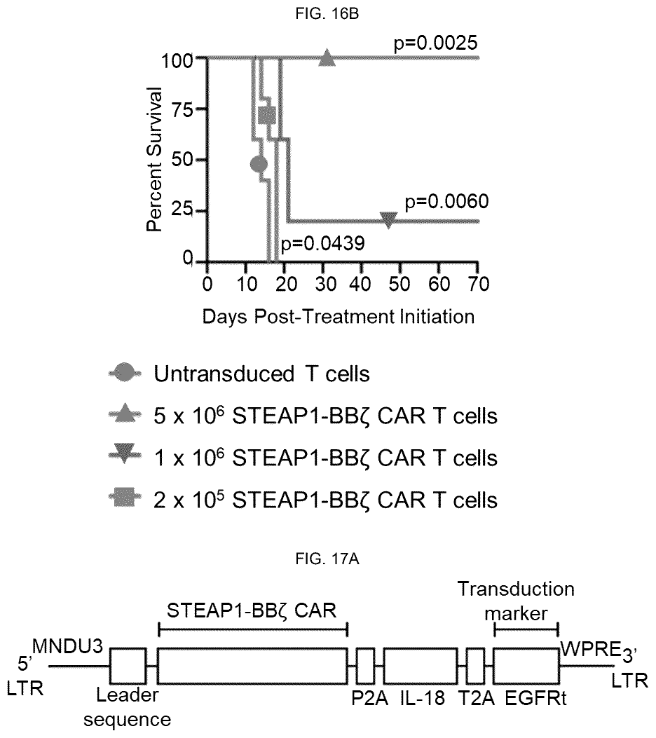

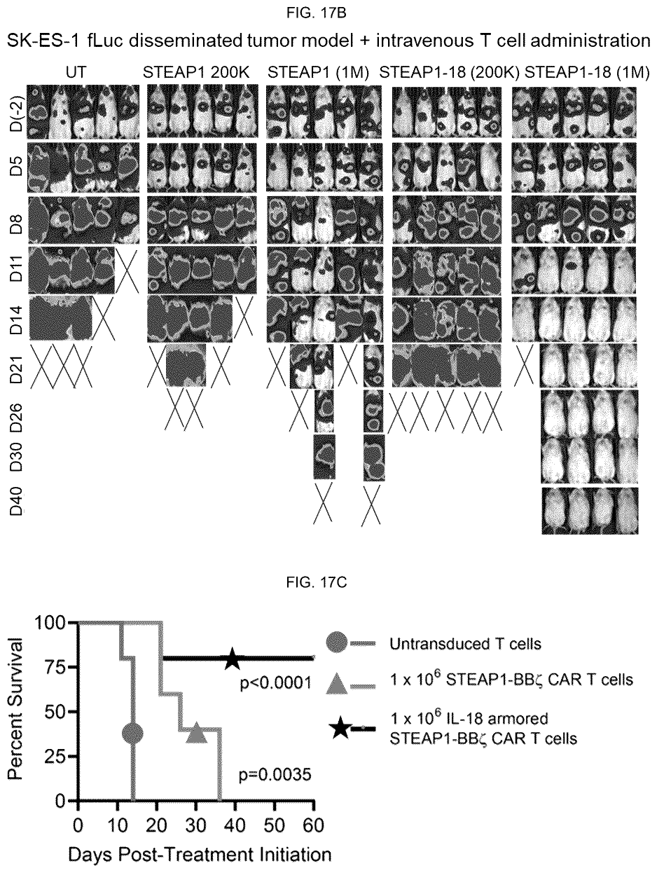

To investigate safety and efficacy of STEAP1-mBBζ CAR T cell therapy, male heterozygous hSTEAP1-KI mice were inoculated with syngeneic RM9-STEAP1-fLuc cells by tail vein injection (FIG. 10A). After confirmation of metastatic colonization by BLI a week later, mice received pre-conditioning cyclophosphamide 100 mg/kg by intraperitoneal injection. A day later, mice were randomized to treatment with either 5×10⁶ untransduced mouse T cells or mouse STEAP1-mBBζ CAR T cells by tail vein injection. All mice that received mouse STEAP1-mBBζ CAR T cells demonstrated a decrease in tumor burden within the first week of treatment initiation based on BLI (FIGS. 10B, 10C). The observed response was short-lived but led to a modest extension of survival (21 days versus 12 days, p=0.0138 by log-rank test, FIG. 10D). Importantly, there were no gross toxicities or premature deaths specifically associated with mouse STEAP1-mBBζ CAR T cell therapy at this dose level where clear evidence of antitumor efficacy was observed. Weight loss associated with increased tumor burden was common to both treatment arms (FIGS. 10E, 10F). Residual tumor deposits collected at the end of the experiment showed human STEAP1 expression with minor regional heterogeneity in mice treated with untransduced mouse T cells (FIG. 10G). On the other hand, all tumors from mice treated with mouse STEAP1-mBBζ CAR T cells demonstrated a striking absence of human STEAP1 expression (FIG. 10H). These findings point to either intrinsic resistance due to pre-existing STEAP1-RM9 tumor cells or adaptive resistance through dynamic STEAP1 antigen loss. Importantly, heterozygous hSTEAP1-KI mice treated with STEAP1-mBBζ CAR T cells demonstrated no obvious tissue disruption or increased infiltration of CD3+ T cells in the prostate (FIGS. 11A, 11B) relative to their counterparts treated with untransduced T cells.

### (XIII) Closing Paragraphs

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

To the extent not explicitly provided herein, coding sequences for proteins disclosed herein and protein sequences for coding sequences disclosed herein can be readily derived from one of ordinary skill in the art.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157 (1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within +2 is preferred, those within +1 are particularly preferred, and those within +0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within +2 is preferred, those within +1 are particularly preferred, and those within +0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42 °C in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50 °C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 °C in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 °C with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in STEAP1-expressing cell lysis in an in vitro cell killing assay, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A genetic construct comprising (i) a sequence encoding interleukin-18 (IL-18), and (ii) a sequence encoding a chimeric antigen receptor (CAR) comprising a STEAP1 binding domain, a spacer, a transmembrane domain, and an intracellular component.

2. A genetic construct comprising (i) a sequence encoding a pro-inflammatory cytokine, and (ii) a sequence encoding a recombinant receptor comprising an extracellular component, an intracellular component, and a transmembrane domain linking the extracellular component to the intracellular component, wherein the extracellular component comprises a STEAP1 binding domain.

3. The genetic construct of claim 2, wherein the pro-inflammatory cytokine comprises interleukin (IL)-18, IL1α, IL-1β, IL-33, IL-36α, IL-36β, or IL-36γ.

4. The genetic construct of claim 2, wherein the pro-inflammatory cytokine comprises a sequence as set forth in any of SEQ ID NOs: 4, 6, 7, 8, 9, 10, 11, 12, or 13, or a sequence having at least 95% sequence identity to the sequence as set forth in any of SEQ ID NOs: 4, 6, 7, 8, 9, 10, 11, 12, or 13.

5. The genetic construct of claim 2, wherein the pro-inflammatory cytokine comprises IL-18.

6. The genetic construct of claim 5, wherein the IL-18 comprises the sequence as set forth in SEQ ID NO: 4 or SEQ ID NO: 6 or a sequence having at least 95% sequence identity to the sequence as set forth in SEQ ID NO: 4 or SEQ ID NO: 6.

7. The genetic construct of claim 5, wherein the IL-18 is encoded by the sequence as set forth in SEQ ID NO: 5 or a sequence having at least 95% sequence identity to the sequence as set forth in SEQ ID NO: 5.

8. The genetic construct of claim 2, wherein the recombinant receptor comprises a chimeric antigen receptor (CAR), an engineered T cell receptor (eTCR), or a hybrid thereof.

9. The genetic construct of claim 2, wherein the recombinant receptor comprises a CAR.

10. The genetic construct of claim 2, wherein the STEAP1 binding domain comprises
a variable heavy chain comprising a complementarity determining region (CDRH) 1 as set forth in SEQ ID NO: 14, a CDRH2 as set forth in SEQ ID NO: 15, and a CDRH3 as set forth in SEQ ID NO: 16, and a variable light chain comprising a CDRL1 as set forth in SEQ ID NO: 17, a CDRL2 as set forth in SEQ ID NO: 18, and a CDRL3 as set forth in SEQ ID NO: 19, according to Kabat;
a variable heavy chain comprising a CDRH1 as set forth in SEQ ID NO: 20, a CDRH2 as set forth in SEQ ID NO: 21, and a CDRH3 as set forth in SEQ ID NO: 16, and a variable light chain comprising a CDRL1 as set forth in SEQ ID NO: 17, a CDRL2 as set forth in SEQ ID NO: 18, and a CDRL3 as set forth in SEQ ID NO: 19, according to Chothia;
a variable heavy chain comprising a CDRH1 as set forth in SEQ ID NO: 22, a CDRH2 as set forth in SEQ ID NO: 23, and a CDRH3 as set forth in SEQ ID NO: 24, and a variable light chain comprising a CDRL1 as set forth in SEQ ID NO: 25, a CDRL2 having the sequence WAS, and a CDRL3 as set forth in SEQ ID NO: 19, according to IMGT;
a variable heavy chain comprising a CDRH1 as set forth in SEQ ID NO: 26, a CDRH2 as set forth in SEQ ID NO: 27, and a CDRH3 as set forth in SEQ ID NO: 24, and a variable light chain comprising a CDRL1 as set forth in SEQ ID NO: 17, a CDRL2 as set forth in SEQ ID NO: 28, and a CDRL3 as set forth in SEQ ID NO: 19, according to North; or
a variable heavy chain comprising a CDRH1 as set forth in SEQ ID NO: 29, a CDRH2 as set forth in SEQ ID NO: 30, and a CDRH3 as set forth in SEQ ID NO: 31, and a variable light chain comprising a CDRL1 as set forth in SEQ ID NO: 32, a CDRL2 as set forth in SEQ ID NO: 33, and a CDRL3 as set forth in SEQ ID NO: 34, according to Contact.

11. The genetic construct of claim 2, wherein the STEAP1 binding domain comprises a variable heavy chain with at least 90% sequence identity to the sequence as set forth in SEQ ID NO: 35 and a variable light chain with at least 90% sequence identity to the sequence as set forth in SEQ ID NO: 36.

12. The genetic construct of claim 2, wherein the STEAP1 binding domain comprises a variable heavy chain that has the sequence as set forth in SEQ ID NO: 35 and a variable light chain that has the sequence as set forth in SEQ ID NO: 36.

13. The genetic construct of claim 2, wherein the STEAP1 binding domain comprises a variable heavy chain that is encoded by a sequence having at least 90% sequence identity to the sequence as set forth in SEQ ID NO: 72 and a variable light chain that is encoded by a sequence having at least 90% sequence identity to the sequence as set forth in SEQ ID NO: 70.

14. The genetic construct of claim 2, wherein the STEAP1 binding domain comprises a variable heavy chain that is encoded by the sequence as set forth in SEQ ID NO: 72 and a variable light chain that is encoded by the sequence as set forth in SEQ ID NO: 70.

15. The genetic construct of claim 2, wherein the STEAP1 binding domain comprises a single chain variable fragment (scFv).

16. The genetic construct of claim 15, wherein the scFv has at least 90% sequence identity to the sequence as set forth in SEQ ID NO: 73 or SEQ ID NO: 75.

17. The genetic construct of claim 15, wherein the scFv has the sequence as set forth in SEQ ID NO: 73 or SEQ ID NO: 75.

18. The genetic construct of claim 15, wherein the scFv is encoded by a sequence having at least 90% sequence identity to the sequence as set forth in SEQ ID NO: 74 or SEQ ID NO: 76.

19. The genetic construct of claim 15, wherein the scFv is encoded by the sequence as set forth in SEQ ID NO: 74 or SEQ ID NO: 76.

20. The genetic construct of claim 2, wherein the extracellular component further comprises a spacer.

21. The genetic construct of claim 20, wherein the spacer comprises a long spacer, an intermediate spacer, or a short spacer.

22. The genetic construct of claim 21, wherein the long spacer is 230 amino acids or less.

23. The genetic construct of claim 21, wherein the long spacer comprises a IgG4 hinge region, IgG4-CH2 domain, and IgG4-CH3 domain.

24. The genetic construct of claim 23, wherein the IgG4 is human IgG4.

25. The genetic construct of claim 21, wherein the long spacer comprises a sequence having at least 95% sequence identity to the sequence as set forth in SEQ ID NO: 87.

26. The genetic construct of claim 21, wherein the long spacer comprises the sequence as set forth in SEQ ID NO: 87.

27. The genetic construct of claim 21, wherein the long spacer is encoded by a sequence having at least 95% sequence identity to the sequence as set forth in SEQ ID NO: 88.

28. The genetic construct of claim 21, wherein the long spacer is encoded by the sequence as set forth in SEQ ID NO: 88.

29. The genetic construct of claim 21, wherein the intermediate spacer comprises a hinge region and an IgG4-CH3 domain.

30. The genetic construct of claim 21, wherein the intermediate spacer comprises the sequence as set forth in SEQ ID NO: 222 or a sequence having at least 95% sequence identity to the sequence as set forth in SEQ ID NO: 222.

31. The genetic construct of claim 21, wherein the intermediate spacer is encoded by the sequence as set forth in SEQ ID NO: 86 or a sequence having at least 95% sequence identity to the sequence as set forth in SEQ ID NO: 86.

32. The genetic construct of claim 21, wherein the short spacer comprises an IgG4 hinge region.

33. The genetic construct of claim 21, wherein the short spacer comprises the sequence as set forth in SEQ ID NO: 77 or a sequence having at least 95% sequence identity to the sequence as set forth in SEQ ID NO: 77.

34. The genetic construct of claim 21, wherein the short spacer is encoded by the sequence as set forth in any of SEQ ID NOs: 78-80 or a sequence having at least 95% sequence identity to the sequence as set forth in any of SEQ ID NOs: 78-80.

35. The genetic construct of claim 2, wherein the intracellular component comprises all or a portion of a CD3ζ signaling domain; all or a portion of a 4-1BB signaling domain, all or a portion of a CD28 signaling domain, all or a portion of the CD3ζ signaling domain and the 4-1BB signaling domain; all or a portion of the CD3ζ signaling domain and all or a portion of the CD28 signaling domain; or all or a portion of the CD3ζ signaling domain, all or a portion of the 4-1BB signaling domain, and all or a portion of the CD28 signaling domain.

36. The genetic construct of claim 2, wherein the intracellular component comprises all or a portion of a CD3ζ signaling domain and all or a portion of a 4-1BB signaling domain.

37. The genetic construct of claim 36, wherein the CD3ζ signaling domain has at least 90% sequence identity to the sequence as set forth in any of SEQ ID NOs: 89, 90, or 223.

38. The genetic construct of claim 36, wherein the CD3ζ signaling domain has the sequence as set forth in any of SEQ ID NOs: 89, 90, or 223.

39. The genetic construct of claim 36, wherein the CD3ζ signaling domain is encoded by a sequence having at least 90% sequence identity to the sequence as set forth in SEQ ID NOs: 91 or 92.

40. The genetic construct of claim 36, wherein the CD3ζ signaling domain is encoded by the sequence as set forth in SEQ ID NOs: 91 or 92.

41. The genetic construct of claim 36, wherein the 4-1BB signaling domain has at least 90% sequence identity to the sequence as set forth in any of SEQ ID NOs: 93, 94, or 95.

42. The genetic construct of claim 36, wherein the 4-1BB signaling domain has the sequence as set forth in any of SEQ ID NOs: 93, 94, or 95.

43. The genetic construct of claim 36, wherein the 4-1BB signaling domain is encoded by a sequence having at least 90% sequence identity to the sequence as set forth in any of SEQ ID NOs: 96, 97, or 98.

44. The genetic construct of claim 36, wherein the 4-1BB signaling domain is encoded by the sequence as set forth in any of SEQ ID NOs: 96, 97, or 98.

45. The genetic construct of claim 2, wherein the transmembrane domain comprises a CD28 transmembrane domain.

46. The genetic construct of claim 45, wherein the CD28 transmembrane domain has at least 90% sequence identity to the sequence as set forth in any of SEQ ID NOs: 99, 100, or 101.

47. The genetic construct of claim 45, wherein the CD28 transmembrane domain has the sequence as set forth in any of SEQ ID NOs: 99, 100, or 101.

48. The genetic construct of claim 45, wherein the CD28 transmembrane domain is encoded by a sequence having at least 90% sequence identity to the sequence as set forth in any of SEQ ID NOs: 102, 103, 104, or 105.

49. The genetic construct of claim 45, wherein the CD28 transmembrane domain is encoded by the sequence as set forth in any of SEQ ID NOs: 102, 103, 104, or 105.

50. The genetic construct of claim 2, wherein the STEAP1 binding domain comprises a STEAP1 scFv, the intracellular component comprises a CD3ζ signaling domain and a 4-1BB signaling domain, and the transmembrane domain comprises a CD28 transmembrane domain.

51. The genetic construct of claim 2, wherein the recombinant receptor comprises a sequence having at least 90% sequence identity to the sequence as set forth in SEQ ID NO: 214.

52. The genetic construct of claim 2, wherein the recombinant receptor comprises the sequence as set forth in SEQ ID NO: 214.

53. The genetic construct of claim 2, wherein the recombinant receptor is encoded by a sequence having at least 90% sequence identity to the sequence as set forth in SEQ ID NO: 215.

54. The genetic construct of claim 2, wherein the recombinant receptor is encoded by the sequence as set forth in SEQ ID NO: 215.

55. The genetic construct of claim 2, further comprising a tag cassette or a suicide switch.

56. The genetic construct of claim 2, wherein the recombinant receptor further comprises a multimerization domain.

57. The genetic construct of claim 2, further comprising a self-cleaving polypeptide.

58. The genetic construct of claim 57, wherein the self-cleaving polypeptide comprises a porcine teschovirus-1 (P2A), Thosea asigna virus (T2A), equine rhinitis A virus (E2A), foot-and-mouth disease virus (F2A), or variants thereof.

59. The genetic construct of claim 57, further comprising a second self-cleaving polypeptide.

60. The genetic construct of claim 59, wherein the second self-cleaving polypeptide comprises a porcine teschovirus-1 (P2A), Thosea asigna virus (T2A), equine rhinitis A virus (E2A), foot-and-mouth disease virus (F2A), or variants thereof.

61. The genetic construct of claim 2, further comprising a transduction marker.

62. The genetic construct of claim 61, wherein the transduction marker is a truncated epidermal growth factor receptor (EGFRt).

63. The genetic construct of claim 62, wherein the EGFRt comprises a sequence having at least 90% sequence identity to the sequence as set forth in SEQ ID NO: 106.

64. The genetic construct of claim 62, wherein the EGFRt comprises the sequence as set forth in SEQ ID NO: 106.

65. The genetic construct of claim 62, wherein the EGFRt is encoded by a sequence having at least 90% sequence identity to the sequence as set forth in SEQ ID NO: 10⁷.

66. The genetic construct of claim 62, wherein the EGFRt is encoded by the sequence as set forth in SEQ ID NO: 10⁷.

67. The genetic construct of claim 2, further comprising a selection cassette.

68. The genetic construct of claim 67, wherein the selection cassette comprises dihydrofolate reductase double mutant (DHFRdm).

69. The genetic construct of claim 2, wherein the genetic construct is encoded by a sequence having at least 90% sequence identity to the sequence as set forth in any of SEQ ID NO: 217, 219, 220, or 221.

70. The genetic construct of claim 2, wherein the genetic construct is encoded by the sequence as set forth in any of SEQ ID NOs: 217, 219, 220, or 221.

71. A nanoparticle encapsulating the genetic construct of claim 2.

72. A cell genetically modified to express the genetic construct of claim 2.

73. The cell of claim 72, wherein the cell is an autologous cell or an allogeneic cell in reference to a subject.

74. The cell of claim 72, wherein the cell is in vivo or ex vivo.

75. The cell of claim 72, wherein the cell is an immune cell.

76. The cell of claim 72, wherein the cell is a T cell, B cell, natural killer (NK) cell, NK-T cell, monocyte/macrophage, hematopoietic stem cells (HSC), or a hematopoietic progenitor cell (HPC).

77. The cell of claim 76, wherein the cell is a T cell selected from a CD3+ T cell, a CD4+ T cell, a CD8+ T cell, a central memory T cell, an effector memory T cell, and/or a naïve T cell.

78. The cell of claim 72, wherein the cell is a CD8+ T cell.

79. The cell of claim 72, wherein the cell is a CD4+ T cell.

80. The cell of claim 72, wherein the cell is non-reactive against cells expressing Steap1b.

81. A population of cells genetically modified to express the genetic construct of claim 2.

82. The population of cells of claim 81, wherein the population of cells comprise autologous cells or allogeneic cells in reference to a subject.

83. The population of cells of claim 81, wherein the population of cells are in vivo or ex vivo.

84. The population of cells of claim 81, wherein the population of cells comprise immune cells.

85. The population of cells of claim 81, wherein the population of cells comprise T cells, B cells, natural killer (NK) cells, NK-T cells, monocytes/macrophages, hematopoietic stem cells (HSC), and/or hematopoietic progenitor cell (HPCs).

86. The population of cells of claim 81, wherein the population of cells comprise CD4+ T cells and CD8+ T cells.

87. The population of cells of claim 81, wherein the population of cells comprise a 1:1 ratio of CD4+ T cells to CD8+ T cells.

88. The population of cells of claim 81, wherein the population of cells are non-reactive against cells expressing Steap 1b.

89. A formulation comprising (i) cells genetically modified to express the genetic construct of claim 2 and (ii) a pharmaceutically acceptable carrier.

90. A method of treating a subject with a STEAP1-related disorder comprising co-expressing a therapeutically effective amount of a STEAP1 recombinant receptor and a pro-inflammatory cytokine within the subject, thereby treating the subject.

91. The method of claim 90, wherein the pro-inflammatory cytokine comprises IL-18, IL1α, IL-1β, IL-33, IL-36α, IL-36β, or IL-36γ.

92. The method of claim 90, wherein the pro-inflammatory cytokine comprises IL-18.

93. The method of claim 90, wherein the STEAP1 recombinant receptor comprise a STEAP1 CAR.

94. The method of claim 93, wherein the STEAP1 CAR comprises an extracellular component comprising a STEAP1 binding domain; an intracellular component comprising a CD3ζ signaling domain and a 4-1BB signaling domain; and a transmembrane domain linking the extracellular component to the intracellular component, wherein the transmembrane domain comprises a CD28 transmembrane domain.

95. The method of claim 90, wherein the co-expressing comprises administering a therapeutically effective amount of the formulation of claim 89 to the subject.

96. The method of claim 90, wherein the subject's STEAP1-related disorder is based on presence of diseased cells expressing STEAP1 at low STEAP1 antigen conditions.

97. The method of claim 96, wherein the low STEAP1 antigen conditions comprise less than 50,000 STEAP1 molecules per diseased cell.

98. The method of claim 96, wherein the low STEAP1 antigen conditions comprise less than 30,000 STEAP1 molecules per diseased cell.

99. The method of claim 96, wherein the low STEAP1 antigen conditions comprise less than 15,000 STEAP1 molecules per diseased cell.

100. The method of claim 96, wherein the low STEAP1 antigen conditions comprise less than 10,000 STEAP1 molecules per diseased cell.

101. The method of claim 96, wherein the low STEAP1 antigen conditions comprise less than 5,000 STEAP1 molecules per diseased cell.

102. The method of claim 96, wherein low STEAP1 antigen conditions comprise less than 2,000 STEAP1 molecules per diseased cell.

103. The method of claim 96, wherein low STEAP1 antigen conditions comprise less than 1,500 STEAP1 molecules per diseased cell.

104. The method of claim 96, further comprising obtaining a sample of the diseased cells and measuring the STEAP1 antigen density levels of the cells.

105. The method of claim 90, wherein the STEAP1-related disorder comprises prostate cancer, the Ewing family of tumors (EFT), bladder cancer, ovarian cancer, or rhabdomyosarcoma.

106. The method of claim 105, wherein the EFT comprises Ewing Sarcoma.

107. The method of claim 106 wherein the Ewing Sarcoma comprises orthotopic Ewing sarcoma or disseminated Ewing sarcoma.

108. The method of claim 90, wherein the STEAP1-related disorder comprises lethal, metastatic castration-resistant prostate cancer.

109. The method of claim 95, wherein the administering a therapeutically effective amount comprises administering intravesically, intravenously, intradermally, intraarterially, intraparenterally, intranodally, intralymphatically, intraperitoneally, intralesionally, intraprostatically, intravaginally, intrarectally, topically, intrathecally, intratumorally, intramuscularly, or subcutaneously.

110. A method of providing an immune response against STEAP1-expressing cells in a subject in need thereof comprising co-expressing a therapeutically effective amount of a STEAP1 recombinant receptor and a pro-inflammatory cytokine within the subject, thereby providing an immune response against STEAP1-expressing cells in the subject.

111. The method of claim 110, wherein the pro-inflammatory cytokine comprises IL-18, IL1α, IL-1β, IL-33, IL-36α, IL-36β, or IL-36γ.

112. The method of claim 110, wherein the pro-inflammatory cytokine comprises IL-18.

113. The method of claim 110, wherein the STEAP1 recombinant receptor comprise a STEAP1 CAR.

114. The method of claim 113, wherein the STEAP1 CAR comprises an extracellular component comprising a STEAP1 binding domain; an intracellular component comprising a CD3ζ signaling domain and a 4-1BB signaling domain; and a transmembrane domain linking the extracellular component to the intracellular component, wherein the transmembrane domain comprises a CD28 transmembrane domain.

115. The method of claim 110, wherein the co-expressing comprises administering a therapeutically effective amount of the formulation of claim 89 to the subject.

116. The method of claim 110, wherein the STEAP1-expressing cells comprise prostate cancer cells, the Ewing family of tumor (EFT) cells, bladder cancer cells, ovarian cancer cells, or rhabdomyosarcoma cells.

117. The method of claim 116, wherein the EFT comprises Ewing Sarcoma.

118. The method of claim 117, wherein the Ewing Sarcoma comprises orthotopic Ewing sarcoma or disseminated Ewing sarcoma.

119. The method of claim 110, wherein the STEAP1-expressing cells comprise prostate cancer cells.

120. The method of claim 119, wherein the prostate cancer cells comprise lethal, metastatic castration-resistant prostate cancer cells.

121. The method of claim 115, wherein the administering a therapeutically effective amount comprises administering intravesically, intravenously, intradermally, intraarterially, intraparenterally, intranodally, intralymphatically, intraperitoneally, intralesionally, intraprostatically, intravaginally, intrarectally, topically, intrathecally, intratumorally, intramuscularly, or subcutaneously.

122. A kit comprising nucleic acids encoding a pro-inflammatory cytokine and nucleic acids encoding a recombinant receptor comprising an extracellular component, an intracellular component, and a transmembrane domain linking the extracellular component to the intracellular component, wherein the extracellular component comprises a STEAP1 binding domain.

123. The kit of claim 122, wherein the pro-inflammatory cytokine comprises interleukin (IL)-18, IL1α, IL-1β, IL-33, IL-36α, IL-36β, or IL-36γ.

124. The kit of claim 122, wherein the pro-inflammatory cytokine comprises the sequence as set forth in any of SEQ ID NOs: 4, 6, 7, 8, 9, 10, 11, 12, or 13 or a sequence having at least 95% sequence identity to the sequence as set forth in any of SEQ ID NOs: 4, 6, 7, 8, 9, 10, 11, 12, or 13.

125. The kit of claim 122, wherein the pro-inflammatory cytokine comprises IL-18.

126. The kit of claim 122, wherein the IL-18 comprises the sequence as set forth in SEQ ID NO: 4 or SEQ ID NO: 6 or a sequence having at least 95% sequence identity to the sequence as set forth in SEQ ID NO: 4 or SEQ ID NO: 6.

127. The kit of claim 125, wherein the IL-18 is encoded by the sequence as set forth in SEQ ID NO: 5 or a sequence having at least 95% sequence identity to the sequence as set forth in SEQ ID NO: 5.

128. The kit of claim 122, wherein the recombinant receptor comprises a chimeric antigen receptor (CAR), an engineered T cell receptor (eTCR), or a hybrid thereof.

129. The kit of claim 122, wherein the recombinant receptor comprises a CAR.

130. The kit of claim 122, wherein the STEAP1 binding domain comprises
a variable heavy chain comprising a complementarity determining region (CDRH) 1 as set forth in SEQ ID NO: 14, a CDRH2 as set forth in SEQ ID NO: 15, and a CDRH3 as set forth in SEQ ID NO: 16, and a variable light chain comprising a CDRL1 as set forth in SEQ ID NO: 17, a CDRL2 as set forth in SEQ ID NO: 18, and a CDRL3 as set forth in SEQ ID NO: 19, according to Kabat;
a variable heavy chain comprising a CDRH1 as set forth in SEQ ID NO: 20, a CDRH2 as set forth in SEQ ID NO: 21, and a CDRH3 as set forth in SEQ ID NO: 16, and a variable light chain comprising a CDRL1 as set forth in SEQ ID NO: 17, a CDRL2 as set forth in SEQ ID NO: 18, and a CDRL3 as set forth in SEQ ID NO: 19, according to Chothia;
a variable heavy chain comprising a CDRH1 as set forth in SEQ ID NO: 22, a CDRH2 as set forth in SEQ ID NO: 23, and a CDRH3 as set forth in SEQ ID NO: 24, and a variable light chain comprising a CDRL1 as set forth in SEQ ID NO: 25, a CDRL2 having the sequence WAS, and a CDRL3 as set forth in SEQ ID NO: 19, according to IMGT;
a variable heavy chain comprising a CDRH1 as set forth in SEQ ID NO: 26, a CDRH2 as set forth in SEQ ID NO: 27, and a CDRH3 as set forth in SEQ ID NO: 24, and a variable light chain comprising a CDRL1 as set forth in SEQ ID NO: 17, a CDRL2 as set forth in SEQ ID NO: 28, and a CDRL3 as set forth in SEQ ID NO: 19, according to North; or
a variable heavy chain comprising a CDRH1 as set forth in SEQ ID NO: 29, a CDRH2 as set forth in SEQ ID NO: 30, and a CDRH3 as set forth in SEQ ID NO: 31, and a variable light chain comprising a CDRL1 as set forth in SEQ ID NO: 32, a CDRL2 as set forth in SEQ ID NO: 33, and a CDRL3 as set forth in SEQ ID NO: 34, according to Contact.

131. The kit of claim 122, wherein the STEAP1 binding domain comprises a variable heavy chain with at least 90% sequence identity to the sequence as set forth in SEQ ID NO: 35 and a variable light chain with at least 90% sequence identity to the sequence as set forth in SEQ ID NO: 36.

132. The kit of claim 122, wherein the STEAP1 binding domain comprises a variable heavy chain that has the sequence as set forth in SEQ ID NO: 35 and a variable light chain that has the sequence as set forth in SEQ ID NO: 36.

133. The kit of claim 122, wherein the STEAP1 binding domain comprises a variable heavy chain that is encoded by a sequence having at least 90% sequence identity to the sequence as set forth in SEQ ID NO: 72 and a variable light chain that is encoded by a sequence having at least 90% sequence identity to the sequence as set forth in SEQ ID NO: 70.

134. The kit of claim 122, wherein the STEAP1 binding domain comprises a variable heavy chain that is encoded by the sequence as set forth in SEQ ID NO: 72 and a variable light chain that is encoded by the sequence as set forth in SEQ ID NO: 70.

135. The kit of claim 122, wherein the STEAP1 binding domain comprises a single chain variable fragment (scFv).

136. The kit of claim 135, wherein the scFv has at least 90% sequence identity to the sequence as set forth in SEQ ID NO: 73 or SEQ ID NO: 75.

137. The kit of claim 135, wherein the scFv has the sequence as set forth in SEQ ID NO: 73 or SEQ ID NO: 75.

138. The kit of claim 135, wherein the scFv is encoded by a sequence having at least 90% sequence identity to the sequence as set forth in SEQ ID NO: 74 or SEQ ID NO: 76.

139. The kit of claim 135, wherein the scFv is encoded by the sequence as set forth in SEQ ID NO: 74 or SEQ ID NO: 76.

140. The kit of claim 122, wherein the extracellular component further comprises a spacer.

141. The kit of claim 140, wherein the spacer comprises a long spacer, an intermediate spacer, or a short spacer.

142. The kit of claim 122, wherein the intracellular component comprises all or a portion of a CD3ζ signaling domain; all or a portion of a 4-1BB signaling domain, all or a portion of a CD28 signaling domain, all or a portion of the CD3ζ signaling domain and the 4-1BB signaling domain; all or a portion of the CD3ζ signaling domain and all or a portion of the CD28 signaling domain; or all or a portion of the CD3ζ signaling domain, all or a portion of the 4-1BB signaling domain, and all or a portion of the CD28 signaling domain.

143. The kit of claim 122, wherein the STEAP1 binding domain comprises a STEAP1 scFv, the intracellular component comprises a CD3ζ signaling domain and a 4-1BB signaling domain, and the transmembrane domain comprises a CD28 transmembrane domain.

144. The kit of claim 122, wherein the recombinant receptor comprises a sequence having at least 90% sequence identity to the sequence as set forth in SEQ ID NO: 214.

145. The kit of claim 122, wherein the recombinant receptor comprises the sequence as set forth in SEQ ID NO: 214.

146. The kit of claim 122, wherein the nucleic acids encoding the recombinant receptor comprise a sequence having at least 90% sequence identity to the sequence as set forth in SEQ ID NO: 215.

147. The kit of claim 122, wherein the nucleic acids encoding the recombinant receptor comprise the sequence as set forth in SEQ ID NO: 215.

148. The kit of claim 122, wherein the nucleic acids encoding the pro-inflammatory cytokine and/or nucleic acids encoding the recombinant receptor are encapsulated within a liposome or polymeric nanoparticle.

149. The kit of claim 122, wherein the nucleic acid encoding the pro-inflammatory cytokine and/or nucleic acids encoding the recombinant receptor are within a viral vector.

150. The kit of claim 149, wherein the viral vector is a retroviral vector.

151. The kit of claim 150, wherein the retroviral vector is a lentiviral vector.

152. The kit of claim 122, further comprising guide RNA.
